(19) United States
(12) Patent Application Publication
(10) Pub. No.: US 2012/0251551 A1
(43) Pub. Date: Oct. 4, 2012

(54) Substituted (Heteroarylmethyl)thiohydantoins

(75) Inventors: Ulrich Lücking, Berlin (DE); Arwed Cleve, Berlin (DE); Bernard Haendler, Berlin (DE); Hortensia Faus Gimenez, Berlin (DE); Silke Köhr, Berlin (DE); Horst Irlbacher, Berlin (DE)

(73) Assignee: BAYER PHARMA AKTIENGESELLSCHAFT, Berlin (DE)

(21) Appl. No.: 13/394,536

(22) PCT Filed: Aug. 28, 2010
(86) PCT No.: PCT/EP2010/005297

§ 371 Date: Jun. 18, 2012

(30) Foreign Application Priority Data
Sep. 11, 2009 (EP) 09075421.9
Feb. 17, 2010 (EP) 10075069.4

(51) Int. Cl. A61K 31/4439; C07D 401/06; C07D 413/14; A61K 31/5377; C07D 417/14; A61K 31/541; A61K 31/551; A61K 31/4545; C07D 403/14; A61K 31/506; C07D 409/14; A61K 31/565; A61K 39/395; A61P 35/00; C07D 401/14

(52) U.S. Cl. 424/158.1; 546/274.1; 514/341; 546/274.4; 544/131; 514/235.8; 544/58.2; 514/227.8; 540/575; 514/218; 546/194; 514/318; 544/331; 514/275; 546/272.4; 514/171

(57) ABSTRACT

The invention relates to substituted (heteroarylmethyl) thiohydantoin compounds of general formula (I) as described and defined herein, and methods for their preparation, their use for the treatment and/or prophylaxis of disorders, and their use for the preparation of medicaments for the treatment and/or prophylaxis of disorders, in particular of prostate cancer.

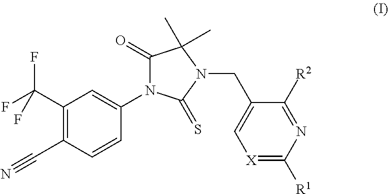

The present invention relates to substituted (heteroarylmethyl)thiohydantoin compounds of general formula (I) as described and defined herein, and methods for their preparation, their use for the treatment and/or prophylaxis of disorders, and their use for the preparation of medicaments for the treatment and/or prophylaxis of disorders, in particular of prostate cancer.

In industrialized countries, prostate cancer is the second main cause of death by cancer in men, after lung cancer. In men over 55 years of age, 4% of all deaths can be attributed to a prostate tumor disorder, and autopsy studies show that in men over 80, close to 70% have prostate cancer. The death rate is still relatively low, but it increases yearly by about 14%. The number of men in whom a prostate tumor was diagnosed has increased by 30% in recent years, which can be attributed less to an increasing number of new disease cases but rather to the fact that the population is generally getting older, that diagnostic processes have improved and that systematic screening programs have been introduced (E. J. Small, D. M. Reese, Curr. Opi. Oncol. 2000, 12, 265-272).

Prostate tumor grows in an androgen-dependent manner. As long as the tumor is locally confined to the prostate, it can be removed by surgical intervention or by radiation therapy, whereby these methods are associated with well-documented risks. In the cases in which the tumor is no longer confined to the prostate capsule and has already formed metastases, it is treated by reduction of the androgen supply to the tumor. This is achieved either surgically by castration or medicinally by treatment with anti-androgens (bicalutamide, cyproterone acetate, flutamide), LHRH-agonists (leuprolide, goserelin, buserelin), LHRH-antagonists (cetrorelix) or 5α-reductase inhibitors (fmasteride). Since the adrenal androgen synthesis remains unaffected in a surgical castration, a combined surgical and medicinal treatment is frequently performed (S. Leewansangtong, E. D. Crawford, Endocrine-Related Cancer 1998, 5, 325-339). These treatments, however, have only temporary success, since after at the latest two years, renewed growth of the tumor is observed, which in most cases is then resistant to current chemical castration therapies (L. J. Denis, K. Griffith, Semin. in Surg. One. 2000, 18, 52-74). Despite intensive research in the last 50 years, no really effective treatment against these advanced stages has emerged. The 5-year survival rate in castration-resistant prostate cancer patients is less than 15%.

There is multiple evidence showing that the androgen receptor plays an important role in the development and the growth of the prostate tumor not only in the early hormone-dependent stages of the tumor progression, but also in the late castration-resistant stage.

The androgen receptor belongs to the family of steroid hormone receptors which act as ligand-dependent transcription factors. The cytoplasmic, unliganded androgen receptor forms a complex with chaperone proteins. Upon binding by androgens, a conformational change takes place, chaperones dissociate from the complex and the liganded androgen receptor translocates into the nucleus. Following binding to cognate DNA response elements located in gene regulatory regions and recruitment of cofactors, the androgen receptor activates or represses a defined subset of target genes (D. J. Lamb et. al. Vitam. Horm. 2001, 62, 199-230).

Identifying compounds with strong and long-lasting anti-androgenic effects will offer new treatment options for prostate cancer patients. Studies with non-steroidal anti-androgens have shown that they have advantages compared to the steroidal compounds and are therefore to be preferred. Thus, with non-steroidal compounds, a more selective action with fewer adverse side-effects can be achieved.

Non-steroidal anti-androgens are described in a number of different patents or patent applications such as for example U.S. Pat. No. 5,434,176, U.S. Pat. No. 5,411,981 or U.S. Re 35,956 (phenylimidazolidine derivatives), U.S. Pat. No. 5,589,497, U.S. Pat. No. 6,162,444, US20040009969 (piperazine derivatives), US20090111864 (diarylhydantoins), EP494819, EP580459, WO95/18794, WO97/00071 (specifically substituted phenyldimethyl hydantoins as well as their imino or thione derivatives), WO00/37430 (phenylalanine, phenyl hydantoins as well as phenyl ureas), WO 01/58855 (aminopropanilides), EP1122242 (substituted cyanophenylpiperazines), WO2006/133567, WO2006/013887, WO2006/028226 or WO2006/124118.

WO2006124118 relates to diarylhydantoin compounds, including diarylthiohydantoins and their use in the treatment of castration-resistant prostate cancer. In contrast to U.S. Pat. No. Re. 35,956 (further discussed below) WO2006124118 does not suggest to substitute the [4-cyano-3-(trifluoro-methyl)phenyl]-substituted thiohydantoin with an aralkyl residue, but instead focuses on aryl residues including heteroaryls such as pyridyl. Both pyridyl derivatives exemplified in WO2006124118, RD82 and RD83, were “no better than bicalutamide for treating prostate cancer” and were therefore ranked into tier 4 which means that, according to the inventors of WO2006124118, pyridyl derivatives of the claimed diarylhydantoins do not possess particularly promising properties. In contrast, more promising results (ranked in tier 1) of WO2006124118 were obtained for compounds which are characterized by substituted phenyl residues.

Also the two anti-androgenic diarylthiohydantoin compounds, RD162 and MDV3100, described by Tran et al. (Science, Vol. 324, (2009), 787-790) are characterized by substituted phenyl residues, which retained antagonistic activity in the setting of increased androgen receptor expression.

U.S. Pat. No. Re. 35,956 generically discloses, i.a., [4-cyano-3-(trifluoromethyl)phenyl]-substituted thiohydantoins or hydantoins with an aralkyl group of up to 12 carbon atoms. According to U.S. Pat. No. Re. 35,956 the term “aralkyl” includes certain alkyls substituted with certain aryls. The term “alkyl” includes alkyl of up to 12 carbon atoms, such as methyl, ethyl, propyl, isopropyl, butyl, isobutyl etc. The term “aryl” is defined to include carbocyclic aryl such as phenyl and naphthyl, and also heterocyclic aryl of 5 to 6 ring members containing at least one heteroatom selected from the group consisting of oxygen, sulfur and nitrogen. U.S. Pat. No. Re. 35,956 specifically mentions 6-ring heteroaryl such as pyridyl, pyrimidinyl, pyridazinyl and pyrazinyl. The “aralkyl” groups of the [4-cyano-3-(trifluoromethyl)phenyl]-substituted hydantoin compounds which are explicitly disclosed in U.S. Pat. No. Re. 35,956 are confined to phenylmethyl (example 26) and the substituted phenylmethyl groups [4-fluorophenyl)methyl] (example 27), [(4-methoxyphenyl)methyl] (example 28) and [[4-(trifluoromethyl)phenyl]methyl] (example 29).

The [4-cyano-3-(trifluoromethyl)phenyl]-substituted thiohydantoin compounds explicitly exemplified in U.S. Pat. No. Re. 35,956 concern unsubstituted alkyl (examples 12, 38, 39, 81), hydroxyalkyl (examples 23, 71, 75, 77), alkoxyalkyl (example 79).

U.S. Pat. No. Re. 35,956 provides data concerning the affinity of selected compounds for the androgen receptor of rats and their anti-androgenic activity in mice, respectively. No data are disclosed showing anti-androgenic activity of these specific compounds against human androgen receptor, neither with respect to the human “wild type” AR (Swiss-Prot Acc. No. P10275, Entry Version 159, Sequence Version 2) nor towards mutated form(s) of the human AR such as W741L or W741C (Hara et al., Cancer Research, 63: 149-153, 2003) or E709Y (Georget et al., Molecular Endocrinology, 20(4): 724-734, 2006). U.S. Pat. No. Re. 35,956 does also not provide any data regarding the potential agonistic properties of the specified compounds. Furthermore, no data are disclosed demonstrating an anti-proliferative action in cells that originate from human prostate cancers (e.g. LNCaP or VCaP cells) or showing a reasonable metabolic stability or clearance of these compounds which make them suitable for pharmaceutical applications, particularly for an effective therapy of prostate cancer.

Some clinical findings have been reported concerning the relationship between cancer relapse after anti-androgen drug administration and androgen receptor mutations.

Androgen receptor mutations were observed in 5 out of 17 patients who experienced relapsed prostate cancer after an endocrine therapy with a combination of flutamide and castration, all of which were missense mutations of the amino acid at position 877 of the androgen receptor (Taplin et al., Cancer Res., 59: 2511-2515, 1999). For these mutants at position 877 some anti-androgen drugs, including flutamide, were found to behave as agonists and to stimulate prostate cancer cell proliferation (Veldscholte et al., Biochem. Biophys. Res. Commun., 173: 534-540, 1990).

Haapala et al. (Lab. Invest., 81: 1647-1651, 2001) described different mutations of the androgen receptor, which were identified in biopsy samples from patients who experienced relapsed prostate cancer after an endocrine therapy with a combination of bicalutamide and surgical castration. Three of the detected mutations were missense mutations (G166S, W741C, M7491) and two were silent polymorphisms. None of the investigated tumors showed an amplification of the androgen receptor. Haapala et al. conclude that different types of androgen receptor alterations in prostate tumors are selected for during various types of hormonal therapy.

Hara et al. (Cancer Research, 63: 149-153, 2003) demonstrated that bicalutamide, which is the most commonly used anti-androgen, acted as an agonist for both the W741C and W741L androgen receptor mutants. The W741C and W741L mutation affect the same codon 741 in the ligand-binding domain of the androgen receptor. In one case codon 741, TGG (tryptophan), is mutated to TGT (cysteine). In the other case it is mutated to TTG (leucine). Within only 6-13 weeks of in vitro exposure to bicalutamide, LNCaP-FGC cells, whose growth had initially been suppressed, came to use bicalutamide as an androgen receptor agonist to survive, due to mutation of the codon 741.

Additional evidence that the W741C mutation causes bicalutamide to act as an agonist was provided through data from a xenograft model (Yoshida et al., Cancer Research, 65: 9611-9616, 2005).

Georget et al. (Molecular Endocrinology, 20(4): 724-734, 2006) demonstrate that the E709Y mutation causes the conversion of bicalutamide into a partial agonist.

Therefore, the identification of anti-androgens that inhibit not only the human “wild type” androgen receptor but also certain mutated forms of the androgen receptor such as the W741L or W741C mutant would presumably be very helpful in treating prostate tumors in various stages, especially in castration-resistant stages, and/or in treating prostate tumors of such patient groups showing a W741L or W741C mutation of the androgen receptor.

Furthermore, the identification of compounds with minimal agonistic activity with respect to the human “wild type” androgen receptor and with potency to antagonize the androgen activity of the human “wild type” androgen receptor is required in order to treat prostate tumors at different stages, particularly in their treatment refractory stages. Preferably, these compounds are also potent in antagonizing the androgen activity of the W741L and/or W741C and/or the E709Y mutated form(s) of the androgen receptor.

In addition to these properties, the desired compounds preferably have an enhanced anti-proliferation effect on prostate tumor cells compared to known compounds and/or show desirable pharmacological properties, such as for example a reasonable metabolic stability or (blood) clearance.

Therefore, the object of the invention is to provide compounds with minimal agonistic activity with respect to the human “wild type” androgen receptor and with high potency to antagonize the androgen activity of the human “wild type” androgen receptor.

A further object of the invention is to provide compounds, which, in addition to the minimal agonistic activity and to the high antagonistic potency against the human “wild type” androgen receptor, are potent to antagonize the androgen activity of the W741L mutated form of the human androgen receptor.

Another object of the invention is to provide compounds, which, in addition to the minimal agonistic activity and to the high antagonistic potency against the human “wild type” androgen receptor, are potent to antagonize the androgen activity of the W741L and/or the W741C and/or the E709Y mutated form of the human androgen receptor and/or show desirable pharmacological properties, such as for example a reasonable metabolic stability or clearance.

Further, it has been shown that the androgen receptor is overexpressed frequently in castration-resistant prostate cancer (Linja M J et al., Cancer Res 2001; 61: 3550-5; Latil A et al, Cancer Res 2001; 61: 1919-26). In addition, ˜30% of castration-resistant prostate cancer carry androgen receptor gene amplification (Visakorpi T et al., Nat Genet 1995; 9: 401-6). Functional evidence that the androgen receptor is involved in the emergence of castration-resistant prostate cancer was presented by Chen and colleagues (Chen C D et al., Nat Med 2004; 10: 33-9) who showed that increase in androgen receptor expression was the only modification consistently associated with the development of antiandrogen therapy resistance and that overexpression of a ectopic androgen receptor was sufficient to transform androgen-dependent prostate cancer cells into androgen-independent ones. Also, different groups (Kokontis J et al., Cancer Res 1994; 54^{.} 1566-73; Waltering K K et al, Cancer Res 2009, 69: 8141-9) have shown previously that adaptation of LNCaP cells to low levels of androgens is associated with increased expression of endogenous androgen receptor. Together with the findings that androgen receptor overexpression is common in castration-resistant prostate cancer, the experimental data suggest that the overexpression of this receptor is a key mechanism for the progression of prostate cancer.

Therefore, the identification of anti-androgens that inhibit the proliferation of cell lines overexpressing the androgen receptor due to an amplified androgen receptor gene would presumably be very helpful in treating prostate tumors in various stages, especially in castration-resistant stages, and/or in treating prostate tumors of such patient groups showing an overexpression of the androgen receptor due to an amplified androgen receptor gene.

Another object of the invention therefore is to provide compounds, which, in addition to the minimal agonistic activity and to the high antagonistic potency against the human “wild type” androgen receptor, are potent to antagonize the androgen activity of the W741L and/or the W741C and/or the E709Y mutated form of the human androgen receptor and which show an anti-proliferative effect against prostate cancer cell lines with amplified androgen receptor gene, such as the VCaP cell line (Korenchuk S et al., In Vivo 2001, 15: 163-8, 2001; Liu W et al., Neoplasia 2008, 10: 897-907).

The present invention relates to compounds of the formula (I)

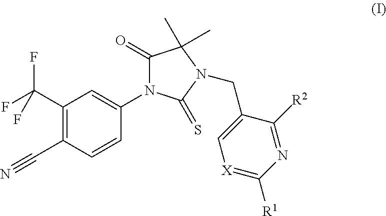

wherein
X means nitrogen or a CH group,
R¹ means a fluorinated C₁-C₃-alkyl-group, a perfluorinated C₁-C₃-alkyl-group, a trifluoromethyl group, an optionally fluorinated C₁-C₄-alkoxy-group, an optionally substituted hydroxy-C₂-C₄-alkoxy-group
   which is substituted with one or two or three substituents selected from the group consisting of methyl, fluorine and trifluoromethyl;
an optionally substituted hydroxypropoxy-group,
   which is substituted with one or two or three substituents selected from the group consisting of methyl, fluorine and trifluoromethyl;
a 2-hydroxy-2-methylpropoxy-group,
an optionally substituted methoxy-C₂-C₄-alkoxy-group,
   which is substituted with one or two or three substituents selected from the group consisting of methyl, fluorine and trifluoromethyl;
an optionally substituted methoxyethoxy-group,
   which is substituted with one or two or three substituents selected from the group consisting of methyl, fluorine and trifluoromethyl;
a (tetrahydro-2H-pyranyl)oxy-group,
an optionally substituted five membered heteroaromatic group selected from the group consisting of pyrrolyl, pyrazolyl, imidazolyl, triazolyl, tetrazolyl, thienyl, oxazolyl, isoxazolyl, furanyl, thiazolyl, oxadiazolyl,
   wherein the five membered heteroaromatic group is substituted with one or two substituents selected from the group consisting of methyl, trifluoromethyl, methoxy-, trifluoromethoxy-, chlorine, fluorine, hydroxy, amino, hydroxymethyl and cyano;
an optionally substituted five, six, or seven membered heterocyclic group selected from pyrrolidinyl, piperidinyl, piperazinyl, morpholinyl, thiomorpholinyl, diazepanyl, tetrahydrofuranyl, pyrrolinyl, imidazolidinyl and oxazepanyl,
   wherein the five, six, or seven membered heterocyclic group is substituted with one or two or three substituents selected from the group consisting of methyl, trifluoromethyl, hydroxymethyl, fluorine, hydroxy, oxo, oxido, imino, C₁-C₄-alkylimino, methylimino, cyanoimino, and cyano;
a residue —O(CH₂)ₙ—Y wherein n=2 or n=3, and Y is an optionally substituted five, six, or seven membered heterocyclic group selected from pyrrolidinyl, piperidinyl, piperazinyl, morpholinyl, thiomorpholinyl, diazepanyl, tetrahydrofuranyl, pyrrolinyl, imidazolidinyl and oxazepanyl,
   wherein the five, six, or seven membered heterocyclic group is substituted with one or two substituents selected from the group consisting of methyl, trifluoromethyl, hydroxymethyl, fluorine, hydroxy, oxo, oxido, imino, C₁-C₄-alkylimino-, and cyano; or
a residue —N═S(═O)R³R⁴, wherein R³ represents an aryl group or a phenyl group and R⁴ represents a C₁-C₄-alkyl or a methyl group;
R² means hydrogen, methyl, amino or fluorine,

or their salts, solvates or salts of solvates.

Compounds according to the invention are the compounds of the formula (I) and the salts, solvates and solvates of the salts thereof, the compounds of the hereinafter recited formulae which are encompassed by formula (I) and the salts, solvates and solvates of the salts thereof, and the compounds which are encompassed by formula (I) and are mentioned hereinafter as exemplary embodiments and the salts, solvates and solvates of the salts thereof, where the compounds which are encompassed by formula (I) and are mentioned hereinafter are not already salts, solvates and solvates of the salts.

The compounds according to the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

If the compounds according to the invention can be in tautomeric forms, the present invention encompasses all tautomeric forms.

Salts which are preferred for the purposes of the present invention are physiologically acceptable salts of the compounds according to the invention. However, salts which are not suitable for pharmaceutical applications per se, but which, for example, can be used for the isolation or purification of the compounds according to the invention, are also comprised.

Physiologically acceptable salts of the compounds according to the invention encompass acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also comprise salts of conventional bases, such as, by way of example and by preference, alkali metal salts (for example sodium and potassium salts), alkaline earth metal salts (for example calcium and magnesium salts) and ammonium salts derived from ammonia or organic amines with 1 to 16 C atoms, such as, by way of example and by preference, ethylamine, diethylamine, triethylamine, ethyldiisopropyl-amine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates is the term used for the purposes of the invention for those forms of the compounds according to the invention which form a complex with solvent molecules by coordination in the solid or liquid state. Hydrates are a special form of solvates in which the coordination takes place with water. Hydrates are preferred as solvates within the scope of the present invention.

In addition, the present invention also encompasses prodrugs of the compounds according to the invention. The term “prodrugs” encompasses compounds which themselves may be biologically active or inactive, but are converted (for example by metabolism or hydrolysis) to compounds according to the invention during their residence time in the body.

For the purposes of the present invention, the substituents have the following meaning, unless otherwise specified:

The term “alkyl” per se and “alk” and “alkyl” in alkoxy, alkylcarbonyl, alkylamino, alkylaminocarbonyl, alkoxycarbonyl, alkoxycarbonylamino and alkylcarbonylamino represent a linear or branched alkyl radical having the number of carbon atoms specifically indicated, e.g. C₁-C₃ one, two or three carbon atoms, C₂-C₄ two, three or four carbon atoms, by way of example and by preference methyl, ethyl, n-propyl, isopropyl, n-butyl, tert-butyl. If the number of carbon atoms is not specifically indicated the term “alkyl” represents a linear or branched alkyl radical having, as a rule, 1 to 6, preferably 1 to 4, especially preferably 1 to 3, carbon atoms, by way of example and by preference methyl, ethyl, n-propyl, isopropyl, n-butyl, tert-butyl, n-pentyl and n-hexyl. Particularly, the alkyl group has 1, 2, 3 or 4 carbon atoms (“C₁-C₄-alkyl”), methyl, ethyl, n-propyl, isopropyl, n-butyl or tert-butyl. Preferably, the alkyl group has 1, 2 or 3 carbon atoms (“C₁-C₃-alkyl”), methyl, ethyl, n-propyl or isopropyl.

The terms “fluorine” and “chlorine” represent halogen atoms selected from fluorine and chlorine, respectively.

The term “fluorinated C₁-C₃-alkyl-” group is to be understood as preferably meaning a linear or branched, saturated, monovalent, hydrocarbon group, in which the term “alkyl” is to be understood as defined supra, in which one or more of the hydrogen atoms is replaced by a fluorine atom or by two, three, four, five, six or seven fluorine atoms. Said fluorinated C₁-C₃-alkyl group is, for example, a —CF₃, a —CHF₂, a —CH₂F, a —CF₂CF₃, or a —CH₂CF₃ group, preferably it is a perfluorinated group or a —CF₃ group.

“Alkoxy” represents by way of example and by preference methoxy, ethoxy, n-propoxy, isopropoxy, n-butoxy, and tert-butoxy. The term “C₁-C₄-alkoxy-” is to be understood as preferably meaning a linear or branched, saturated, monovalent, hydrocarbon group of formula —O-alkyl, in which the term “alkyl” is to be understood as defined supra, e.g. a methoxy, ethoxy, n-propoxy, isopropoxy, n-butoxy, iso-butoxy, tert-butoxy, sec-butoxy or an isomer thereof. Particularly, the “C₁-C₄-alkoxy” group is a methoxy, an ethoxy, a propoxy or a 2-methylpropoxy group.

The term “fluorinated C₁-C₄-alkoxy-” group is to be understood as preferably meaning a linear or branched, saturated, monovalent, hydrocarbon group, in which the term “alkoxy” is to be understood as defined supra, and in which one or more of the hydrogen atoms is replaced by a fluorine atom or by two, three, four, five, six, seven, eight or nine fluorine atoms. Said fluorinated C₁-C₄-alkoxy-group is, for example, an —OCF₃, an —OCHF₂, an —OCH₂F, an —OCH₂CHF₂, an —OCH₂CH₂F, an —OCF₂CF₃, an O—CF₂CHF₂ or an —OCH₂CF₃ group.

The term “hydroxy-C₂-C₄-alkoxy” is to be understood as preferably meaning a linear or branched, saturated, monovalent C₂-C₄-alkoxy group, as defined supra, in which one or more of the hydrogen atoms is replaced by a hydroxy group. Said C₂-C₄-hydroxyalkoxy group is, for example a 2-hydroxyethoxy, a 3-hydroxypropoxy, a 2-hydroxypropoxy, a 2,3-dihydroxypropoxy, a 2-hydroxy-2-methylpropoxy group, preferably a 2-hydroxy-2-methylpropoxy group.

The term “methoxy-C₂-C₄-alkoxy-” is to be understood as preferably meaning a linear or branched, saturated, monovalent C₂-C₄-alkoxy group, as defined supra, in which one of the hydrogen atoms is replaced by a methoxy group. Said “methoxy-C₂-C₄-alkoxy-” group is, for example a 2-methoxyethoxy, a 3-methoxypropoxy, a 2-methoxypropoxy, preferably a 2-methoxyethoxy group.

“Heteroaromatic group” represents an aromatic, monocyclic radical. A “five membered heteroaromatic group” has 5 ring atoms, and up to 4, up to 3, preferably up to 2, hetero atoms from the series consisting of S, O and N, by way of example pyrazolyl, thienyl, furanyl, pyrrolyl, thiazolyl, oxazolyl, isoxazolyl, imidazolyl, oxadiazolyl, triazolyl, tetrazolyl. Preferred are imidazolyl, thienyl, triazolyl, tetrazolyl or pyrazolyl groups. Also preferred are 1H-pyrazol-1-yl, 1H-pyrazol-4-yl, 1H-pyrazol-5-yl, 1H-imidazol-1-yl, 1H-1,2,3-triazol-1-yl, 2H-1,2,3-triazol-2-yl, 1H-1,2,4-triazol-1-yl, 1H-tetrazol-1-yl or thien-2-yl groups. Most preferred is a 1H-imidazol-1-yl group.

The term “heterocyclic group” represents a monocyclic, nonaromatic heterocyclic radical having, as a rule, 4 to 7, preferably 5 to 6, ring atoms and up to 3, preferably up to 2, hetero atoms and/or hetero groups from the series consisting of N, O, S, SO, SO₂, SO(NH). The heterocyclyl radicals can be saturated or partially unsaturated. Preferred are 5- to 7-membered monocyclic saturated heterocyclyl radicals having up to two hetero atoms from the series consisting of O, N and S. The following may be mentioned by way of example: tetrahydropyranyl, pyrrolidinyl, pyrrolinyl, piperidinyl, morpholinyl, thiomorpholinyl, piperazinyl, diazepanyl, oxazepanyl, tetrahydropyranyl, imidazolidinyl. Preferably, the heterocyclic group represents a morpholinyl, thiomorpholinyl or a pyrrolidinyl group. Also preferred are morpholin-4-yl, thiomorpholin-4-yl, imidazolidin-1-yl or pyrrolidin-1-yl groups.

The term “aryl” is to be understood as preferably meaning a monovalent, aromatic monocyclic hydrocarbon ring, particularly a ring having 6 carbon atoms (a “C₆-aryl” group), preferably a phenyl group.

The term “C₁-C₄”, as used throughout this text, e.g. in the context of the definition of C₁-C₄-alkyl, C₁-C₄-alkoxy, C₁-C₄-alkylimino, is to be understood as meaning an alkyl group having a finite number of carbon atoms of 1 to 4, i.e. 1, 2, 3 or 4 carbon atoms. It is to be understood further that said term “C₁-C₄” is to be interpreted as any sub-range comprised therein, e.g. C₁-C₄, C₁-C₃, C₁-C₂, C₂-C₄, C₂-C₃, C₃-C₄.

The term “C₁-C₃”, as used throughout this text, e.g. in the context of the definition of perfluorinated C₁-C₃-alkyl is to be understood as meaning an alkyl group having a finite number of carbon atoms of 1 to 3, i.e. 1, 2 or 3 carbon atoms. It is to be understood further that said term “C₁-C₃” is to be interpreted as any sub-range comprised therein, e.g. C₁-C₃, C₁-C₂, C₂-C₃.

Similarly, as used herein, the term “C₂-C₄”, as used throughout this text, e.g. in the context of the definition of “hydroxy-C₂-C₄-alkoxy” or “methoxy-C₂-C₄-alkoxy”, is to be understood as meaning an alkyl group having a finite number of carbon atoms of 2 to 4, i.e. 2, 3 or 4 carbon atoms. It is to be understood further that said term “C₂-C₄” is to be interpreted as any sub-range comprised therein, e.g. C₂-C₄, C₂-C₃, C₃-C₄.

As used herein, the term “one or more times”, e.g. in the definition of the substituents of the compounds of the general formulae of the present invention, is understood as meaning one, two, three, four or five times, particularly one, two, three or four times, more particularly one, two or three times, even more particularly one or two times.

Where the plural form of the word compounds, salts, hydrates, solvates and the like, is used herein, this is taken to mean also a single compound, salt, isomer, hydrate, solvate or the like.

When radicals in the compounds according to the invention are substituted, the radicals may be monosubstituted or polysubstituted, unless otherwise specified. Within the scope of the present invention, the meanings of all radicals which occur repeatedly are independent from one another. A substitution by one, two or three identical or different substituents is preferred. The substitution by one substituent is very specially preferred.

Preferred compounds of the formula (I) are those, wherein
X means nitrogen or a CH group,
R′ means a fluorinated C₁-C₃-alkyl-group, a perfluorinated C₁-C₃-alkyl-group, a trifluoromethyl group, a C₁-C₄-alkoxy-group, a C₁-C₂-alkoxy-group, a
   an optionally substituted hydroxy-C₂-C₄-alkoxy-group,
      which is substituted with one or two substituents selected from the group consisting of methyl and fluorine;
   an optionally substituted hydroxypropoxy-group,
      which is substituted with one or two substituents selected from the group consisting of methyl and fluorine;
   a 2-hydroxy-2-methylpropoxy-group, a 2,2-difluoro-3-hydroxypropoxy-group,
   a methoxy-C₂-C₄-alkoxy-group, a methoxyethoxy-group,
   a (tetrahydro-2H-pyranyl)oxy-group,
   an optionally substituted five membered heteroaromatic group selected from the group consisting of pyrazolyl, triazolyl, tetrazolyl, thienyl and imidazolyl,
      wherein the five membered heteroaromatic group is substituted with one or two substituents selected from the group consisting of methyl, trifluoromethyl, hydroxymethyl or chlorine,
   an optionally substituted five or six or seven membered heterocyclic group selected from pyrrolidinyl, piperidinyl, morpholinyl, thiomorpholinyl, diazepanyl, imidazolidinyl,
   wherein the five or six or seven membered heterocyclic group is substituted with one or two or three substituents selected from the group consisting of methyl, hydroxymethyl, imino, methylimino, cyanoimino, oxido and oxo;
   a residue —O(CH₂)ₙ—Y wherein n=2 and Y is a morpholin-4-yl group or a 2-oxoimidazolidin-1-yl group; or
   a residue —N═S(═O)R³R⁴, wherein R³ represents a phenyl group and R⁴ represents a methyl group;
R² means hydrogen, methyl, or amino,

or their salts, solvates or salts of solvates.

Further preferred compounds of the formula (I) are those, wherein
X means nitrogen or a CH group,
R¹ means an optionally substituted hydroxy-C₂-C₄-alkoxy-group,
   which is substituted with one or two substituents selected from the group consisting of methyl and fluorine;
a methoxy-C₂-C₄-alkoxy-group,
a (tetrahydro-2H-pyranyl)oxy-group,
an optionally substituted five membered heteroaromatic group selected from the group consisting of pyrazolyl, triazolyl, tetrazolyl and imidazolyl,
   wherein the five membered heteroaromatic group is substituted with one or two substituents selected from the group consisting of methyl, trifluoromethyl, hydroxymethyl or chlorine;
an optionally substituted five or six membered heterocyclic group selected from pyrrolidinyl, morpholinyl, thiomorpholinyl, imidazolidinyl,
   wherein the five or six membered heterocyclic group is substituted with one or two or three substituents selected from the group consisting of methyl, imino, methylimino, cyanoimino, oxido and oxo; or
a residue —O(CH₂)ₙ—Y wherein n=2 and Y is a 2-oxoimidazolidin-1-yl group;
R² means hydrogen or methyl,

or their salts, solvates or salts of solvates.

Further preferred compounds of the formula (I) are those, wherein
X means a CH group,
R¹ means a fluorinated C₁-C₃ alkyl-group, a perfluorinated C₁-C₃-alkyl-group, particularly preferred a trifluoromethyl group,
   a C₁-C₄-alkoxy-group, a C₁-C₂-alkoxy group, a methoxy group,
   an optionally substituted hydroxy-C₂-C₄-alkoxy group,
      which is substituted with one or two substituents selected from the group consisting of methyl and fluorine;
   an optionally substituted hydroxypropoxy-group,
      which is substituted with one or two substituents selected from the group consisting of methyl and fluorine;
   a 2-hydroxy-2-methylpropoxy group, a 2,2-difluoro-3-hydroxypropoxy-group,
   a methoxy-C₂-C₄-alkoxy-group, a methoxyethoxy-group,
   a (tetrahydro-2H-pyranyl)oxy-group,
   an optionally substituted five membered heteroaromatic group selected from the group consisting of pyrazolyl, triazolyl, tetrazolyl, and imidazolyl,
      wherein the five membered heteroaromatic group is substituted with methyl,
   an optionally substituted five or six membered heterocyclic group selected from piperidinyl, morpholinyl, thiomorpholinyl, imidazolidinyl and pyrrolidinyl,
      wherein the five or six membered heterocyclic group is substituted with one or two or three substituents selected from the group consisting of methyl, hydroxymethyl, imino, methylimino, oxido and oxo; or
   a residue —O(CH₂)ₙ—Y wherein n=2 and Y is a morpholin-4-yl group or a 2-oxoimidazolidin-1-yl group;
R² means hydrogen, methyl or amino,

or their salts, solvates or salts of solvates.

Further preferred compounds of the formula (I) are those, wherein
X means a CH group,
R¹ means a fluorinated C₁-C₃-alkyl-group, a perfluorinated C₁-C₃-alkyl-group, a trifluoromethyl group,
   an optionally substituted hydroxy-C₂-C₄-alkoxy-group,
      which is substituted with one or two substituents selected from the group consisting of methyl and fluorine;
   an optionally substituted hydroxypropoxy-group,
      which is substituted with methyl or fluorine;
   a 2-hydroxy-2-methylpropoxy-group, a 2,2-difluoro-3-hydroxypropoxy-group,
   a methoxy-C₂-C₄-alkoxy-group, a methoxyethoxy-group,
   a (tetrahydro-2H-pyranyl)oxy-group, a (tetrahydro-2H-pyran-4-yl)oxy-group,
   an optionally substituted five membered heteroaromatic group selected from the group consisting of triazolyl, tetrazolyl, and imidazolyl,
      wherein the five membered heteroaromatic group is substituted with methyl;
   an optionally substituted five or six membered heterocyclic group selected from pyrrolidinyl, imidazolidinyl, morpholinyl and thiomorpholinyl,
      wherein the five or six membered heterocyclic group is substituted with one or two or three substituents selected from the group consisting of methyl, imino, methylimino, oxido and oxo; or
   a residue —O(CH₂)ₙ—Y wherein n=2 and Y is a morpholin-4-yl group or a 2-oxoimidazolidin-1-yl group;
R² means hydrogen or methyl,

or their salts, solvates or salts of solvates.

Further particularly preferred compounds of the formula (I) are those, wherein
X means nitrogen or a CH group,
R¹ means an optionally substituted hydroxy-C₂-C₄-alkoxy-group,
   which is substituted with a methyl group;
a (tetrahydro-2H-pyranyl)oxy-group,
an optionally substituted five membered heteroaromatic group selected from the group consisting of pyrazolyl and imidazolyl,
   wherein the five membered heteroaromatic group is substituted with one or two substituents selected from the group consisting of methyl, trifluoromethyl, hydroxymethyl or chlorine;
an optionally substituted five or six membered heterocyclic group selected from pyrrolidinyl, morpholinyl, thiomorpholinyl, imidazolidinyl,
   wherein the five or six membered heterocyclic group is substituted with one or two or three substituents selected from the group consisting of methyl, imino, methylimino, oxido and oxo; or
a residue —O(CH₂)ₙ—Y wherein n=2 and Y is a 2-oxoimidazolidin-1-yl group;
R² means hydrogen or methyl,

or their salts, solvates or salts of solvates.

Further particularly preferred compounds of the formula (I) are those, wherein
X means nitrogen or a CH group,
R¹ an optionally substituted hydroxy-C₂-C₄-alkoxy-group,
   which is substituted with a methyl group;
an optionally substituted hydroxypropoxy-group,
   which is substituted with a methyl group;
a 2-hydroxy-2-methylpropoxy-group,
an optionally substituted imidazolyl group,
   wherein the imidazolyl group is substituted with a trifluoromethyl group,
R² means hydrogen,

or their salts, solvates or salts of solvates.

In a particularly preferred embodiment the invention concerns compounds of the formula (I),

wherein X means a CH group.

In another preferred embodiment the invention relates to compounds of the formula (I), wherein
R¹ means an optionally substituted hydroxypropoxy-group,
   which is substituted with one or two substituents selected from the group consisting of methyl and fluorine.

In another preferred embodiment the invention relates to compounds of the formula (I), wherein
R¹ means an optionally substituted hydroxypropoxy-group, which is substituted with a methyl group.

In a particularly preferred embodiment the invention relates to compounds of the formula (I), wherein
R′ means a 2-hydroxy-2-methylpropoxy-group.

In another preferred embodiment the invention relates to compounds of the formula (I), wherein
R¹ means an optionally substituted five membered heteroaromatic group selected from the group consisting of pyrazolyl and imidazolyl,
   wherein the five membered heteroaromatic group is substituted with one substituent selected from the group consisting of methyl, trifluoromethyl, hydroxymethyl or chlorine.

In another preferred embodiment the invention relates to compounds of the formula (I), wherein R¹ means an optionally substituted imidazolyl group, wherein the imidazolyl group is substituted with a trifluoromethyl group.

In a particularly preferred embodiment the invention relates to compounds of the formula (I), wherein R¹ means an optionally substituted 1H-imidazol-1-yl group, wherein the 1H-imidazol-1-yl group is substituted with a trifluoromethyl group.

In another particularly preferred embodiment the invention relates to compounds of the formula (I), wherein R¹ means a 4-(trifluoromethyl)-1H-imidazol-1-yl group.

In another particularly preferred embodiment the invention relates to compounds of the formula (I), wherein R² means hydrogen.

The definitions of radicals stated individually in the respective combinations, or preferred combinations, are also replaced as desired by definitions of radicals of other combinations, independently of the respective combinations detailed.

Very specially preferred are combinations of two or more of the abovementioned preferred ranges.

In particular, subjects of this invention are the following compounds:
4-(3-{[6-(1H-Imidazol-1-yl)pyridin-3-yl]methyl}-4,4-dimethyl-5-oxo-2-thioxoimidazolidin-1-yl)-2-(trifluoromethyl)benzonitrile,
4-(4,4-Dimethyl-5-oxo-2-thioxo-3-{[6-(trifluoromethyl)pyridin-3-yl]methyl}imidazolidin-1-yl)-2-(trifluoromethyl)benzonitrile,
4-[4,4-Dimethyl-3-({6-[2-(morpholin-4-yl)ethoxy]pyridin-3-yl}methyl)-5-oxo-2-thioxoimidazolidin-1-yl]-2-(trifluoromethyl)benzonitrile,
4-(4,4-Dimethyl-3-{[6-(morpholin-4-yl)pyridin-3-yl]methyl}-5-oxo-2-thioxoimidazolidin-1-yl)-2-(trifluoromethyl)benzonitrile,
4-(4,4-Dimethyl-5-oxo-3-{[6-(tetrahydro-2H-pyran-4-yloxy)pyridin-3-yl]methyl}-2-thioxoimidazolidin-1-yl)-2-(trifluoromethyl)benzonitrile,
4-(3-{[4-Amino-2-(morpholin-4-yl)pyrimidin-5-yl]methyl}-4,4-dimethyl-5-oxo-2-thioxoimidazolidin-1-yl)-2-(trifluoromethyl)benzonitrile,
4-(4,4-Dimethyl-3-{[6-(2-methylmorpholin-4-yl)pyridin-3-yl]methyl}-5-oxo-2-thioxoimidazolidin-1-yl)-2-(trifluoromethyl)benzonitrile,
4-{3-[(6-Methoxypyridin-3-yl)methyl}-4,4-dimethyl-5-oxo-2-thioxoimidazolidin-1-yl]-2-(trifluoromethyl)benzonitrile,
4-(3-{[6-(1-Imino-1-oxido-1λ⁶-thiomorpholin-4-yl)pyridin-3-yl]methyl}-4,4-dimethyl-5-oxo-2-thioxoimidazolidin-1-yl)-2-(trifluoromethyl)benzonitrile,
4-(3-{[6-(2-Hydroxy-2-methylpropoxy)pyridin-3-yl]methyl}-4,4-dimethyl-5-oxo-2-thioxoimidazolidin-1-yl)-2-(trifluoromethyl)benzonitrile,
4-(3-{[6-(2-Methoxyethoxy)pyridin-3-yl]methyl}-4,4-dimethyl-5-oxo-2-thioxoimidazolidin-1-yl)-2-(trifluoromethyl)benzonitrile,
4-(4,4-Dimethyl-3-{[6-(4-methyl-1,4-diazepan-1-yl)pyridin-3-yl]methyl}-5-oxo-2-thioxoimidazolidin-1-yl)-2-(trifluoromethyl)benzonitrile,
4-(4,4-Dimethyl-3-{[2-methyl-6-(trifluoromethyl)pyridin-3-yl]methyl}-5-oxo-2-thioxoimidazolidin-1-yl)-2-(trifluoromethyl)benzonitrile,
4-[3-({6-[4-(Hydroxymethyl)piperidin-1-yl]pyridin-3-yl}methyl)-4,4-dimethyl-5-oxo-2-thioxoimidazolidin-1-yl]-2-(trifluoromethyl)benzonitrile,
4-(4,4-Dimethyl-3-{[6-(2-methyl-1H-imidazol-1-yl)pyridin-3-yl]methyl}-5-oxo-2-thioxoimidazolidin-1-yl)-2-(trifluoromethyl)benzonitrile,
4-(3-{[6-(4,4-Dimethyl-2-oxopyrrolidin-1-yl)pyridin-3-yl]methyl}-4,4-dimethyl-5-oxo-2-thioxoimidazolidin-1-yl)-2-(trifluoromethyl)benzonitrile,
4-(3-{[2-(1H-Imidazol-1-yl)pyrimidin-5-yl]methyl}-4,4-dimethyl-5-oxo-2-thioxoimidazolidin-1-yl)-2-(trifluoromethyl)benzonitrile,
4-(4,4-Dimethyl-3-{[6-(1-methyl-1H-pyrazol-4-yl)pyridin-3-yl]methyl}-5-oxo-2-thioxoimidazolidin-1-yl)-2-(trifluoromethyl)benzonitrile,
4-(4,4-Dimethyl-3-{[6-(4-methyl-1H-imidazol-1-yl)pyridin-3-yl]methyl}-5-oxo-2-thioxoimidazolidin-1-yl)-2-(trifluoromethyl)benzonitrile,
4-(4,4-Dimethyl-3-{[6-(1-methyl-1H-pyrazol-5-yl)pyridin-3-yl]methyl}-5-oxo-2-thioxoimidazolidin-1-yl)-2-(trifluoromethyl)benzonitrile,
4-(3-{[6-(4-Chloro-2-methyl-1H-imidazol-1-yl)pyridin-3-yl]methyl}-4,4-dimethyl-5-oxo-2-thioxoimidazolidin-1-yl)-2-(trifluoromethyl)benzonitrile,
4-[4,4-Dimethyl-3-({6-[1-(methylimino)-1-oxido-1λ⁶-thiomorpholin-4-yl]pyridin-3-yl}methyl)-5-oxo-2-thioxoimidazolidin-1-yl]-2-(trifluoromethyl)benzonitrile,
4-[4,4-Dimethyl-5-oxo-2-thioxo-3-({6-[4-(trifluoromethyl)-1H-imidazol-1-yl]pyridin-3-yl}methyl)imidazolidin-1-yl]-2-(trifluoromethyl)benzonitrile,
4-(4,4-Dimethyl-5-oxo-3-{[6-(thien-2-yl)pyridin-3-yl]methyl}-2-thioxoimidazolidin-1-yl)-2-(trifluoromethyl)benzonitrile,
4-(4,4-Dimethyl-5-oxo-3-{[6-(2-oxoimidazolidin-1-yl)pyridin-3-yl]methyl}-2-thioxoimidazolidin-1-yl)-2-(trifluoromethyl)benzonitrile,
4-{4,4-Dimethyl-3-[6-{[methyl(oxido)phenyl-λ⁶-sulfanylidene]amino}pyridin-3-yl)methyl]-5-oxo-2-thioxoimidazolidin-1-yl}-2-(trifluoromethyl)benzonitrile,
4-(4,4-Dimethyl-3-{[6-(5-methyl-1H-pyrazol-1-yl)pyridin-3-yl]methyl}-5-oxo-2-thioxoimidazolidin-1-yl)-2-(trifluoromethyl)benzonitrile,
4-(3-{[6-(2,2-Difluoro-3-hydroxypropoxy)pyridin-3-yl]methyl}-4,4-dimethyl-5-oxo-2-thioxoimidazolidin-1-yl)-2-(trifluoromethyl)benzonitrile,
4-(4,4-Dimethyl-5-oxo-2-thioxo-3-{[6-(1H-1,2,3-triazol-1-yl)pyridin-3-yl]methyl}imidazolidin-1-yl)-2-(trifluoromethyl)benzonitrile,
4-(4,4-Dimethyl-5-oxo-2-thioxo-3-{[6-(2H-1,2,3-triazol-2-yl)pyridin-3-yl]methyl}imidazolidin-1-yl)-2-(trifluoromethyl)benzonitrile,
4-(4,4-Dimethyl-5-oxo-3-{[6-(1H-tetrazol-1-yl)pyridin-3-yl]methyl}-2-thioxoimidazolidin-1-yl)-2-(trifluoromethyl)benzonitrile,
4-(3-{[6-(4,5-Dichloro-1H-imidazol-1-yl)pyridin-3-yl]methyl}-4,4-dimethyl-5-oxo-2-thioxoimidazolidin-1-yl)-2-(trifluoromethyl)benzonitrile,
4-[4,4-Dimethyl-5-oxo-2-thioxo-3-({6-[3-(trifluoromethyl)-1H-1,2,4-triazol-1-yl]pyridin-3-yl}methyl)imidazolidin-1-yl]-2-(trifluoromethyl)benzonitrile,
4-[3-({6-[4-(Hydroxymethyl)-1H-imidazol-1-yl]pyridin-3-yl}methyl)-4,4-dimethyl-5-oxo-2-thioxoimidazolidin-1-yl]-2-(trifluoromethyl)benzonitrile,
4-[4,4-Dimethyl-5-oxo-3-({6-[2-(2-oxoimidazolidin-1-yl)ethoxy]pyridin-3-yl}methyl)-2-thioxoimidazolidin-1-yl]-2-(trifluoromethyl)benzonitrile,
{4-[5-({3-[4-Cyano-3-(trifluoromethyl)phenyl]-5,5-dimethyl-4-oxo-2-thioxoimidazolidin-1-yl}methyl)pyridin-2-yl]-1-oxido-1λ⁶-thiomorpholin-1-ylidene}cyanamide,
4-(3-{[6-(2-Hydroxy-2-methylpropoxy)-2-methylpyridin-3-yl]methyl}-4,4-dimethyl-5-oxo-2-thioxoimidazolidin-1-yl)-2-(trifluoromethyl)benzonitrile,
4-(4,4-Dimethyl-3-{[6-(5-methyl-1H-tetrazol-1-yl)pyridin-3-yl]methyl}-5-oxo-2-thioxoimidazolidin-1-yl)-2-(trifluoromethyl)benzonitrile,
   or a salt, solvate or solvate of a salt thereof.

Another subject of the present invention is the compound (R)-4-{4,4-Dimethyl-3-[(6-{[methyl(oxido)phenyl-λ⁶-sulfanylidene]amino}pyridin-3-yl)methyl]-5-oxo-2-thioxoimidazolidin-1-yl}-2-(trifluoromethyl)benzonitrile.

In a particularly preferred embodiment the present invention concerns 4-[4,4-Dimethyl-5-oxo-2-thioxo-3-({6-[4-(trifluoromethyl)-1H-imidazol-1-yl]pyridin-3-yl}methyl)imidazolidin-1-yl]-2-(trifluoromethyl)benzonitrile.

In another particularly preferred embodiment the present invention concerns 4-(3-{[6-(2-Hydroxy-2-methylpropoxy)pyridin-3-yl]methyl}-4,4-dimethyl-5-oxo-2-thioxoimidazolidin-1-yl)-2-(trifluoromethyl)benzonitrile.

The invention furthermore relates to a method for the preparation of the compounds of formula (I) according to the invention, in which method an intermediate compound of general formula (2)

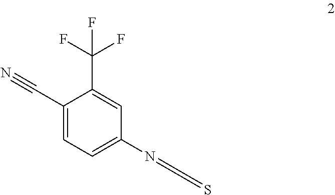

is allowed to react (=is reacted) with a compound of general formula (5),

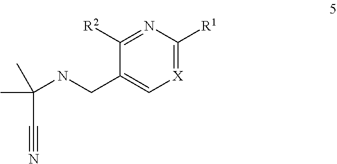

in which X, R¹ and R² are as defined for the compound of general formula (I),
thus providing a compound of general formula (6)

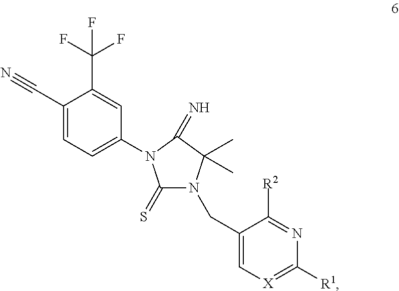

which is then hydrolyzed to the compounds of general formula (I) and the resulting compounds of the formula (I) are optionally, if appropriate, reacted with the corresponding (i) solvents and/or (ii) bases or acids to the solvates, salts and/or solvates of the salts thereof.

The reaction of compound of general formula (2) with the compound of general formula (5) can be performed in aprotic solvents, particularly in tetrahydrofuran, N,N-dimethylformamide or dimethylsulfoxide or mixtures of these solvents, preferably in tetrahydrofuran or N,N-dimethylformamide. The reaction is performed at temperatures ranging from room temperature (=20° C.) to the boiling point of the solvent. The reaction can be performed in the presence of a suitable base, particularly triethylamine or diisopropylethylamine, preferably with triethylamine. The reaction is preferably completed after 1 to 24 hours of reaction time.

The hydrolysis of compounds of general formula (6) to the desired compounds of formula (I) is performed in protic solvents, preferably in methanol or ethanol by adding a suitable acid, such as diluted solutions of hydrogen chloride, or sulfuric acid preferably a 4N solution of hydrogen chloride. The reaction is performed at temperatures ranging from room temperature (=20° C.) to the boiling point of the solvent. The reaction is preferably completed after 1 to 24 hours of reaction time.

The invention furthermore relates to a method for the preparation of the compounds of formula (6), in which method an intermediate compound of general formula (2)

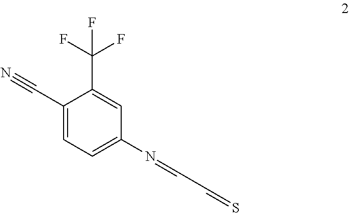

is allowed to react (=is reacted) with a compound of general formula (5)

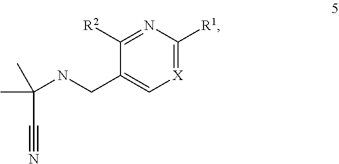

in which X, R¹ and R² are as defined for the compound of general formula (I),
thus providing a compound of general formula (6)

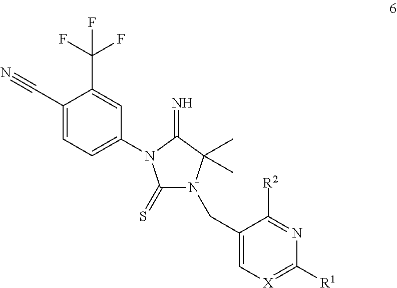

The abovementioned definitions of radicals which have been detailed in general terms or in preferred ranges also apply to the end products of the formula (I) and, analogously, to the starting materials or intermediates required in each case for the preparation.

The preparation of the compounds according to the invention can be illustrated by the following synthesis scheme:

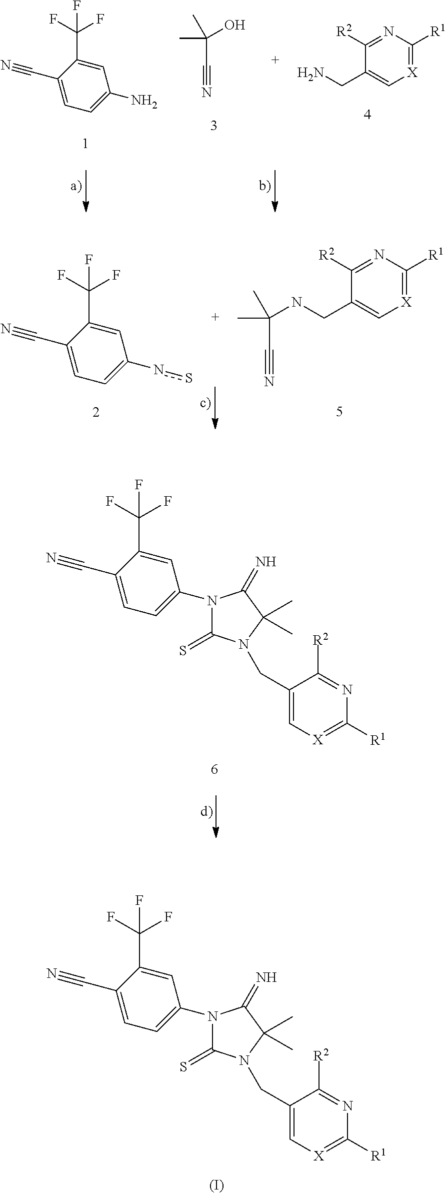

Starting from 4-Amino-2-(trifluoromethyl)benzonitrile (1), the corresponding isothiocyanate (2) may be synthesized utilizing known methodology (Katritzky et al. Comprehensive Heterocyclic Chemistry; Permagon Press: Oxford UK (1984). March. Advanced Organic Chemistry, 3^{rd} Ed.; John Wiley New York (1985)). For example, 4-isothiocyanato-2-(trifluoromethyl)benzonitrile (2) is available as described below from the reaction of 4-Amino-2-(trifluoromethyl)benzonitrile (1) with thiophosgene in tetrahydrofuran at room temperature (=20° C.). Alternatively, 4-isothiocyanato-2-(trifluoromethyl)benzonitrile is commercially available (e.g. Fluorochem, Oakwood, UK).

Reaction of acetone cyanhydrin (3) and amines (4) yields aminoisobutyronitriles (5) (see for example: a) Bucherer et al, Chemische Berichte 1906, 39, 992; b) Cleve et al, US 2004/0009969). The reaction can be run for example as described below in tetrahydrofuran (THF) or N,N-dimethylformamide (DMF) at room temperature (=20° C.) using molecular sieves. Another possibility is the reaction of compounds 3 and 4 without solvent at higher temperatures, for example at 80° C., in the presence of magnesium sulfate (Jung et al, US 2007/0254933). Aminoisobutyronitriles (5) can be reacted with isothiocyanate (2) to give compounds of type 6 (Cleve et al, US 2004/0009969). The reaction can be performed for example using solvents like tetrahydrofuran or N,N-dimethylformamide in the presence of a suitable base like triethylamine at higher temperatures. Finally, compounds of type 6 can be hydrolyzed to the desired compounds of formula (I) (Cleve et al, US 2004/0009969). The reaction can be run for example in a solvent like methanol, adding a 4 N solution of hydrogen chloride at room temperature (=20° C.).

Amines of type 4 are either commercially available or easily accessible via established synthetic methods.

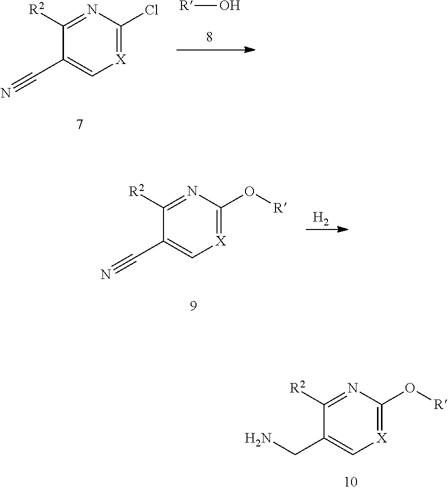

For example, reaction of 2-chloro-pyridine or 2-chloro-pyrimidine derivatives of type 7 with suitable alcohol derivatives of type 8 yields compounds of type 9. C—O bond forming reactions of this type can be performed for example as described in this invention in solvents like dimethylsulfoxide or N,N-dimethylformamide in the presence of a base like sodium hydride at temperatures in the range of 0° C. to 70° C. (see for example: Arienti et al, US 2005/70550). Finally, hydrogenation of compounds of type 9 using for example Raney Nickel as a catalyst yields the desired product 10 (see for example: Forrest et al, J. Chem. Soc. 1948, 1939).

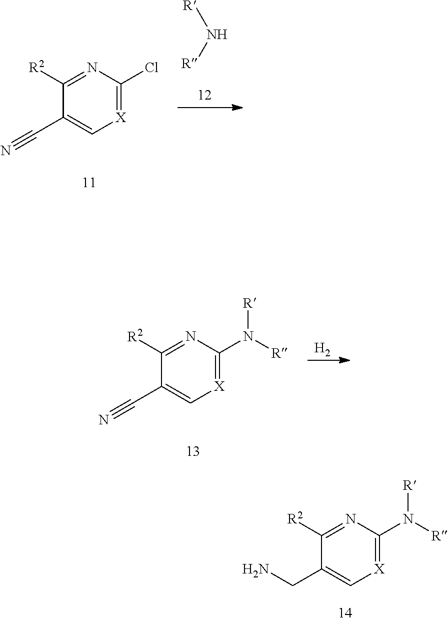

Reaction of 2-chloro-pyridine or 2-chloro-pyrimidine derivatives of type 11 with suitable amine derivatives of type 12 yields compounds of type 13. C—N bond forming reactions of this type can be performed for example as described in this invention in solvents like dimethylsulfoxide or N,N-dimethylformamide in the presence of a base like diisopropylethylamine at temperatures in the range of room temperature (=20° C.) to 100° C. (see for example: Hammond et al, WO 2005/005399). Finally, hydrogenation of compounds of type 13 using for example Raney Nickel as a catalyst yields the desired product 14 (see for example: Nettekoven, US 2006/122187).

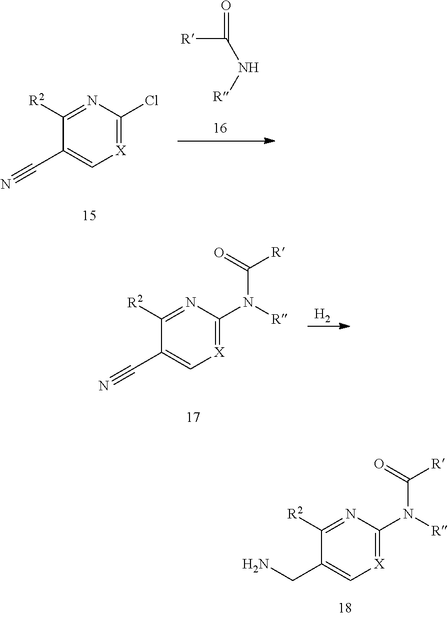

Reaction of 2-chloro-pyridine or 2-chloro-pyrimidine derivatives of type 15 with suitable amide derivatives of type 16 yields compounds of type 17. C—N bond forming reactions of this type can be performed for example as described in this invention in solvents like dimethylsulfoxide, N,N-dimethylformamide or toluene in the presence of a base like sodium hydride at temperatures in the range of 70° C. to 100° C.

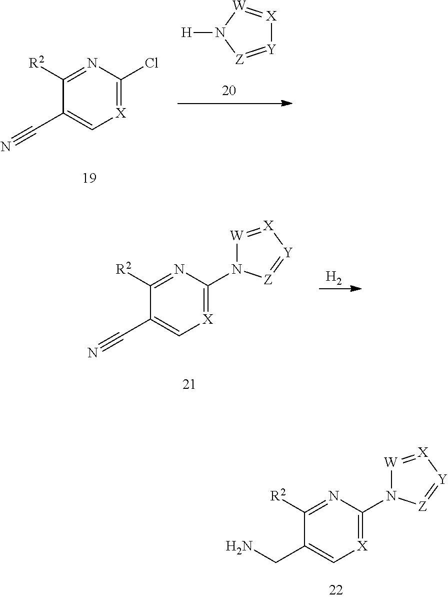

Reaction of 2-chloro-pyridine or 2-chloro-pyrimidine derivatives of type 19 with suitable five membered heteroaromatic compounds having an N—H group of type 20 yields compounds of type 21. C—N bond forming reactions of this type can be performed for example as described in this invention in solvents like dimethylsulfoxide or N,N-dimethylformamide in the presence of a base like potassium carbonate at temperatures in the range of ambient temperatures to 120° C. (see for example: Hirano, US 2004/19045). Finally, hydrogenation of compounds of type 21 using for example Raney Nickel as a catalyst yields the desired product 22.

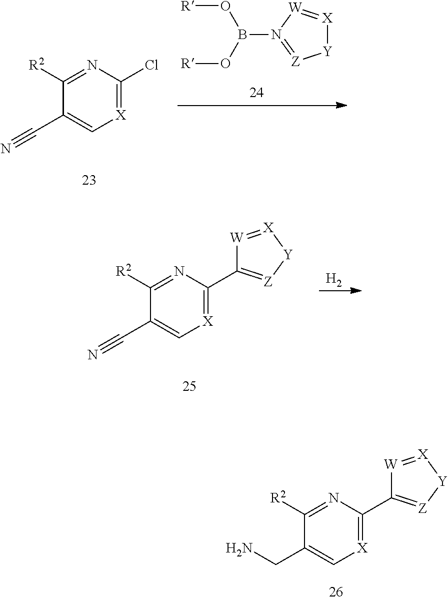

Reaction of 2-chloro-pyridine or 2-chloro-pyrimidine derivatives of type 23 with suitable five membered heteroaromatic compounds having a boronic acid or ester moiety of type 24 yields compounds of type 25. C—C bond forming reactions of this Suzuki reaction type can be performed for example as described in this invention in solvents like 1,2-dimethoxyethane or N,N-dimethylformamide in the presence of a catalyst like tetrakis(triphenylphosphine)palladium(0) and a base like sodium carbonate at temperatures in the range of 90° C. to 140° C. (see for example: Berdini et al, WO 2005/061463).

The compounds according to the invention show a valuable pharmacological and pharmacokinetic spectrum of action which could not have been predicted.

They are therefore suitable for use as medicaments for the treatment and/or prophylaxis of disorders in humans and animals.

Within the scope of the present invention, the term “treatment” includes prophylaxis.

The pharmaceutical activity of the compounds according to the invention can be explained by their action as anti-androgens with minimal agonistic activity with respect to the human “wild type” androgen receptor and with high potency to antagonize the androgen activity of the human “wild type” androgen receptor.

Furthermore, the compounds according to the invention are potent to antagonize the androgen activity of the W741L and/or the W741C and/or the E709Y mutated form(s) of the human androgen receptor.

Further, the compounds according to the invention show desirable pharmacological properties. For example, the compounds of Examples 1, 2, 9, 10, 13, 17, 18, 21, 23 and 24 showed a calculated hepatic in vivo blood clearance (CL) in human liver microsomes of 0.26 l/h/kg (example 1), 0.39 l/h/kg (example 2), 0.48 l/h/kg (example 9), 0.35 l/h/kg (example 10), 0.19 l/h/kg (example 13), 0.09 l/h/kg (example 17), 0.11 l/h/kg (example 18), 0.40 l/h/kg (example 21), 1.0E-4 l/h/kg (example 23) and 0.40 l/h/kg (example 24), respectively. In the context of the present invention, the calculated hepatic in vivo blood clearance (CL) is preferably determined according to the method described below (“Determination of metabolic stability in vitro (including calculation of hepatic in vivo blood clearance (CL) and of maximal oral bioavailability (Fmax)”).

Furthermore, the compounds according to the invention mediate an anti-proliferative activity in prostate tumor cell lines such as LNCaP and/or VCaP. For example, the compounds of Examples 1, 2, 3, 4, 5, 7, 8, 9, 10, 13, 15, 16, 18, 19, 20, 22 and 23 showed an inhibition IC₅₀ (LNCaP) of 59 nM (example 1), 314 nM (example 2), 127 nM (example 3), 117 nM (example 4), 200 nM (example 5), 118 nM (example 7), 120 nM (example 8), 303 nM (example 9), 283 nM (example 10), 124 nM (example 13), 116 nM (example 15), 121 nM (example 16), 117 nM (example 18), 96 nM (example 19), 46 nM (example 20), 135 nM (example 22) and 160 nM (example 23), respectively. For example, the compounds of Examples 4, 7, 8 and 10 showed an inhibition IC_{50 (VCaP) of} 124 nM (example 4), 106 nM (example 7), 92 nM (example 8) and 229 nM (example 10), respectively. In the context of the present invention, the IC₅₀ with respect to prostate tumor cell lines such as LNCaP and/or VCaP is preferably determined according to the methods described below (“Proliferation assay with LNCaP cells”; “Proliferation assay with VCaP cells”).

The present invention relates to the use of the compounds according to the invention for the treatment and/or prophylaxis of disorders (=for use in the treatment and/or prophylaxis of disorders), preferably of hyper-proliferative disorders, preferably of androgen receptor mediated disorders or of androgen-sensitive disorders, whose progress is aided by activation of androgen receptors. The compounds of the invention can be utilized to inhibit, block, reduce, decrease cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof which is effective to treat the disorder.

Hyper-proliferative disorders include but are not limited to e.g., solid tumors, such as cancers of the prostate, breast, respiratory tract, brain, male and female reproductive organs, digestive tract, urinary tract, eye, liver, skin, head and neck, thyroid, parathyroid and their distant metastases, and blood tumors such as lymphomas, sarcomas, and leukemias. They also include benign prostate hyperplasia (BPH), and hyperplasias affecting the skin such as psoriasis and keloids. Furthermore, the compounds according to the invention are used for the treatment and/or prophylaxis of disorders such as acne, seborrhea, hirsutism, androgenic alopecia, male baldness, precocious puberty and polycystic ovarian syndrome.

Tumors of the male reproductive organs include, but are not limited to prostate, testicular and epididymal cancer. Examples of prostate cancers include, but are not limited to in situ carcinoma, prostatic intraepithelial neoplasia, adenocarcinoma, metastasized cancer, hormone-resistant prostate cancer and castration-resistant prostate cancer. In particular, the present invention relates to the use of the compounds according to the invention for use in the treatment and/or prophylaxis of androgen-dependent prostate cancer or of castration-resistant prostate cancer and/or for use in the treatment and/or prophylaxis of benign prostate hyperplasia (BPH).

In particular, the present invention relates to the use of the compounds according to the invention for use in the treatment and/or prophylaxis of castration-resistant prostate cancer, in particular of the chemotherapy-naïve form of castration-resistant prostate cancer and/or of the chemotherapy-resistant form of castration-resistant prostate cancer.

Furthermore, the present invention relates to the use of the compounds according to the invention for the treatment and/or prophylaxis of castration-resistant prostate cancer, which is characterized by the overexpression of the androgen receptor due to an amplified androgen receptor gene.

Furthermore, the present invention relates to the use of the compounds according to the invention for the treatment and/or prophylaxis of castration-resistant prostate cancer, which is characterized by the W741L and/or W741C and/or E709Y mutation of the androgen receptor.

In the context of the present invention the term “androgen-dependent prostate cancer” is to be understood as meaning a prostate tumor that responds to the treatment with GnRH (LHRH) ligands and anti-androgen(s) and is measured by the decrease in blood PSA level.

In the context of the present invention the term “castration-resistant prostate cancer” is to be understood as a prostate tumor that progresses after androgen ablation therapy, for instance after treatment with GnRH (LHRH) ligands and anti-androgen(s). This is usually measured by a rise in blood PSA level or velocity.

The term “chemotherapy-naive form” of a castration-resistant prostate cancer is to be understood as meaning that no treatment with chemotherapeutic agents was performed after emergence of resistance against androgen ablation therapy.

The term “chemotherapy-resistant form” of a castration-resistant prostate cancer is to be understood as meaning a prostate cancer, which shows no response to chemotherapy treatment such as taxanes or mitoxantrone.

In context of the present invention the term “benign prostate hyperplasia (BPH)” refers to the hyperplasia of prostatic stromal and epithelial cells interfering with urine flow.

Tumors of the female reproductive organs include, but are not limited to uterine cancer, to cervical, ovarian, vaginal, and vulvar cancer, as well as sarcoma of the uterus. Benign hyperproliferative disorders of the endometrium (endometriosis) and of the myometrium (uterine fibroids, uterine leiomyomata) are included as well.

Examples of breast cancers include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

A further subject matter of the present invention is the use of the compounds according to the invention for the treatment and/or prophylaxis of disorders, in particular of the disorders mentioned above.

A further subject matter of the present invention are the compounds according to the invention for use in a method for the treatment and/or prophylaxis of hyper-proliferative disorders mentioned above, in particular of prostate cancer and/or of androgen-dependent prostate cancer and/or of castration-resistant prostate cancer and/or of benign prostate hyperplasia (BPH).

In particular, the present invention concerns the compounds according to the invention for use in a method for the treatment and/or prophylaxis of castration-resistant prostate cancer, in particular of the chemotherapy-naïve form of castration-resistant prostate cancer and/or of the chemotherapy-resistant form of castration-resistant prostate cancer.

A further subject matter of the present invention is the use of the compounds according to the invention in the manufacture of a medicament for the treatment and/or prophylaxis of disorders, in particular the disorders mentioned above.

A further subject matter of the present invention is a method for the treatment and/or prophylaxis of disorders, in particular the disorders mentioned above, using an effective amount of the compounds according to the invention.

A further subject matter of the present invention are compositions, preferably pharmaceutical combinations, or medicaments comprising at least one compound according to the invention and at least one or more further active ingredients, in particular for the treatment and/or prophylaxis of the disorders mentioned above. Suitable active ingredients for combinations which may be mentioned by way of example and by preference are:
LHRH (luteinizing hormone-releasing hormone) agonists (=GnRH (gonadotropin-releasing hormone) agonists),
LHRH (luteinizing hormone-releasing hormone) antagonists (=GnRH (gonadotropin-releasing hormone) antagonists),
C(17,20)-lyase inhibitors,
5-alpha-reductase inhibitors type I,
5-alpha-reductase inhibitors type II,
cytostatic agents,
VEGF (Vascular Endothelial Growth Factor)-Kinase inhibitors
antigestagens,
antiestrogens,
EGF Antibodies,
estrogens, or
other AR (androgen receptor) antagonists.

For example, the compounds of this invention can be combined with known anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating agents, growth factor inhibitors, cell cycle inhibitors, enzyme inhibitors, topoisomerase inhibitors, biological response modifiers, or anti-hormones.

The additional pharmaceutical agent can be aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, histrelin, hycamtin, hydrocortone, eyrthro-hydroxynonyladenine, hydroxyurea, ibritumomab tiuxetan, idarubicin, ifosfamide, interferon alpha, interferon-alpha 2, interferon alfa-2A, interferon alfa-2B, interferon alfa-n1, interferon alfa-n3, interferon beta, interferon gamma-1a, interleukin-2, intron A, iressa, irinotecan, kytril, lentinan sulfate, letrozole, leucovorin, leuprolide, leuprolide acetate, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, Mesna, methotrexate, metvix, miltefosine, minocycline, mitomycin C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel, pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, sparfosic acid, stem-cell therapy, streptozocin, strontium-89 chloride, synthroid, tamoxifen, tamsulosin, tasonermin, tastolactone, taxane, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, sorafenib, avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-llinked lintuzumab, EM-1421, HyperAcute vaccine, tucotuzumab celmoleukin, galiximab, HPV-16-E7, Javelin-prostate cancer, Javelin-melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131I-chTNT-1/B. Monoclonal antibodies useful as the protein therapeutic include, but are not limited to, muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

The compounds according to the invention can act systemically and/or locally. For this purpose, they can be administered in a suitable way, such as, for example, by the oral, parenteral, pulmonal, nasal, sublingual, lingual, buccal, rectal, dermal, transdermal, conjunctival or otic route, or as an implant or stent.

For these administration routes, it is possible to administer the compounds according to the invention in suitable application forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (coated or uncoated, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbally) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for the other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems (such as plasters, for example), milk, pastes, foams, dusting powders, implants or stents.

The compounds according to the invention can be converted into the stated administration forms. This can take place in a manner known per se by mixing with inert, nontoxic, pharmaceutically suitable adjuvants. These adjuvants include, inter alia, carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavour- and/or odour-masking agents.

The present invention furthermore provides medicaments comprising at least one compound according to the invention, usually together with one or more inert, nontoxic, pharmaceutically suitable adjuvants, and their use for the treatment and/or prophylaxis of hyper-proliferative disorders as mentioned above.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average dosage per day on oral administration is from about 0.05 to 10 mg/kg, preferably 0.1 to 4 mg/kg of body weight. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will range from 0.1 to 1000 mg, preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

It may nevertheless be necessary, where appropriate, to deviate from the amounts mentioned, depending on the body weight, the administration route, the individual response to the active ingredient, the mode of preparation and the time or interval over which administration takes place. Thus, in some cases, it may be sufficient to make dosages with less than the aforementioned minimal amount, whereas in other cases the upper limit mentioned must be exceeded. In the event of administration of larger amounts, it may be advisable to divide these into a plurality of individual doses over the day.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The compounds of the present invention can be used in particular in therapy and prevention, i.e. prophylaxis, of tumor growth and metastases, especially in solid tumors of all indications and stages with or without pre-treatment of the tumor growth.

The invention also concerns a method of controlling hyper-proliferative disorders such as e.g. prostate cancer in humans and animals by administering an effective amount of at least one compound of the present invention or of a medicament of the present invention.

The present invention also concerns a method of treating hyper-proliferative disorders or a hyper-proliferative disorder in a mammal, which comprises administering to a mammal in need of such treatment an effective amount of a compound of the present invention or of a medicament of the present invention or of a composition according to the invention.

The percentage data in the following tests and examples are percentages by weight unless otherwise indicated; parts are parts by weight. Solvent ratios, dilution ratios and concentration data of liquid/liquid solutions are in each case based on volume.

### Assays:

The in vitro pharmacological properties of the compounds can be determined according to the following assays:

### Cell-Based Transactivation Assay for Wild-Type Human Androgen Receptor

PC-3 cells (Kaighn et al., Invest. Urol. 17:16-23, 1979) stably transfected with the human androgen receptor (Swiss-Prot Acc. No. P10275, Entry Version 159, Sequence Version 2) and a reporter plasmid based on pGL4.14 (#E6691, Promega Corporation, Madison, Wis., USA) containing the MMTV promoter (Cato et al., EMBO J. 6:363-8, 1987) were used. They were grown in 5% charcoal-stripped medium and seeded at a concentration of 1000 cells per well in a 384-well plate. For determination of antagonistic activity, the plate additionally contained the compound to be tested at a concentration range varying from 5.12×10⁻¹² to 1×10⁻⁵ M. The assay was performed in presence of 1×10⁻¹⁰ R1881 (also known as methyltrienolone). After overnight incubation at 37° C. in a 5% CO₂ atmosphere, 15 μl of Steady Glo Lysis and Detection reagent (Steady Glo Luciferase assay system E2550 from Promega Corporation. Madison, Wis., USA) were added. IC₅₀ was calculated for anti-androgenic activity as well as percentage inhibition in presence of 2 μM compound in comparison to non-stimulated luciferase signal. Agonistic activity was determined using the same concentration range of compound in the absence of R1881 by measuring Luciferase activity as above. EC₅₀ was calculated for androgenic activity. Assay/read plates were used (Polystyrol; 384, NV-white cell culture plates; Perkin Elmer).

### Cell-Based Transactivation Assay for Human Androgen Receptor Mutant W741L or W741C

PC-3 cells (Kaighn et al., Invest. Urol. 17:16-23, 1979) were grown in 5% charcoal-stripped medium and seeded at a concentration of 10000 cells per well in a 96-well plate. They were transiently transfected with a pSG5-derived plasmid (#216201 from Stratagene, La Jolla, Calif., USA) coding for the human androgen receptor W741L or W741C mutant Mara et al., Cancer Research, 63: 149-153, 2003) and an MMTV-Luciferase reporter plasmid based on pGL4.14 14 (#E6691, Promega Corporation, Madison, Wis., USA). The compound to be tested was added at a concentration range varying from 1×10⁻⁹ to 1×10 M together with 1×10⁻¹⁰ R1881. After 24 h incubation at 37° C. in a 5% CO₂ atmosphere, 100 μl of Steady Glo Lysis and Detection reagent (Steady Glo Luciferase assay system E2550 from Promega Corporation. Madison, Wis., USA) were added. Antagonistic activity was determined by measuring Luciferase activity in a Victor 3 Luminometer (PerkinElmer, Waltham, Mass., USA) using the Steady Glo Luciferase Assay (E2550, Promega). IC₅₀ values were calculated for anti-androgenic activity.

### Cell-Based Transactivation Assay for Human Androgen Receptor Mutant E709Y

PC-3 cells (Kaighn et al., Invest. Urol. 17:16-23, 1979) were grown in 5% charcoal-stripped medium and seeded at a concentration of 10000 cells per well in a 96-well plate. They were transiently transfected with a pSG5-derived plasmid (#216201 from Stratagene, La Jolla, Calif., USA) coding for the human androgen receptor E709Y mutant (Georget et al., Molecular Endocrinology, 20(4): 724-734, 2006) and an MMTV-Luciferase reporter plasmid based on pGL4.14 14 (#E6691, Promega Corporation, Madison, Wis., USA). The compound to be tested was added at a concentration range varying from 1×10⁻⁹ to 1×10⁻⁶ M together with 1×10⁻¹⁰ R1881. After 24 h incubation at 37° C. in a 5% CO₂ atmosphere, 100 μl of Steady Glo Lysis and Detection reagent (Steady Glo Luciferase assay system E2550 from Promega Corporation. Madison, Wis., USA) were added. Antagonistic activity was determined by measuring Luciferase activity in a Victor 3 Luminometer (PerkinElmer, Waltham, Mass., USA) using the Steady Glo Luciferase Assay (E2550, Promega). IC₅₀ values were calculated for anti-androgenic activity.

Proliferation Assay with LNCaP Cells

LNCaP cells (Horoszewicz et al., in “Models for Prostate Cancer” (ed. G. P. Murphy), Alan R. Liss, New York 1981, p. 115-132; Horoszewicz et al., Cancer Res. 43:1809-1818, 1983) were seeded at 2000 cells/well in 96-well plates in RPMI (F1235, Biochrom AG, Berlin, Germany) without phenol red supplemented with 5% charcoal-stripped serum. After 3 days, the cells were treated with R1881) (1×10⁻¹⁰) and compound (day 0). Cell number was determined by Alamar Blue (DAL1100, Invitrogen, Life Technologies, Lohne, Germany) staining (2.5 h) at day 0 and day 7. Fluorescence was determined in Victor3 (Excitation 530 nm; emission 590 nm). Stimulated growth was defined as the signal measured at day 7 for cells treated only with R1881. Basal level was defined as the signal measured at day 7 for cells grown without R1881.

Proliferation Assay with VCaP Cells

VCaP cells ((Korenchuk et al., In Vivo 15: 163-168, 2001)) were seeded at 16 000 cells/well in 96-well plates in DMEM (F0445, Biochrom AG, Berlin, Germany) with phenol red supplemented with 10% charcoal-stripped serum. After 1 day, the cells were treated with R1881 (1×10⁻¹⁰) and compound (day 0). Cell number was determined by Alamar Blue (DAL1100, Invitrogen, Life Technologies, Lohne, Germany) staining (2.5 h) at day 0 and day 7. Fluorescence was determined in Victor3 (Excitation 530 nm; emission 590 nm). Stimulated growth was defined as the signal measured at day 7 for cells treated only with R1881. Basal level was defined as the signal measured at day 7 for cells grown without R1881.

The in vitro pharmacokinetic properties of the compounds can be shown in the following assay:

### Determination of Metabolic Stability In Vitro (Including Calculation of Hepatic In Vivo Blood Clearance (CL) and of Maximal Oral Bioavailability (Fmax))

The metabolic stability of test compounds in vitro was determined by incubating them at 1 μM with a suspension of human liver microsomes in 100 mM phosphate buffer, pH7.4 (NaH₂PO₄×H₂O+Na₂HPO₄×2H₂O) at a protein concentration of 0.5 mg/ml and at 37° C. The reaction was activated by adding a co-factor mix containing 1.2 mg NADP, 3 IU glucose-6-phosphate dehydrogenase, 14.6 mg glucose-6-phosphate and 4.9 mg MgCl₂ in phosphate buffer, pH 7.4. Organic solvent in the incubations was limited to <0.2% dimethylsulfoxide (DMSO) and <1% methanol. During incubation, the microsomal suspensions were continuously shaken and aliquots were taken at 2, 8, 16, 30, 45 and 60 min, to which equal volumes of cold methanol were immediately added. Samples were freezed at −20° C. over night, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, specific liver weight and microsomal protein content the hepatic in vivo blood clearance (CL) and the maximal oral bioavailability (Fmax) were calculated for the different species. The following parameter values were used: Liver blood flow—1.3 l/h/kg human; specific liver weight—21 g/kg human; microsomal protein content—40 mg/g.

With the described assay only phase-I metabolism of microsomes is reflected, e.g. typically oxidoreductive reactions by cytochrome P450 enzymes and flavin mono-oxygenases (FMO) and hydrolytic reactions by esterases (esters and amides).

The example testing experiments described herein serve to illustrate the present invention and the invention is not limited to the examples given.

### EXAMPLE 1

### 4-(3-{[6-(1H-Imidazol-1-yl)pyridin-3-yl]methyl}-4,4-dimethyl-5-oxo-2-thioxoimidazolidin-1-yl)-2-(trifluoromethyl)benzonitrile

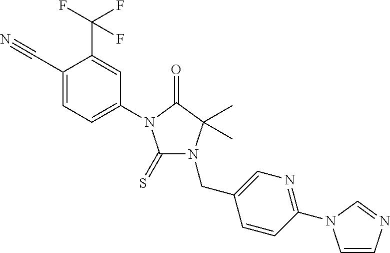

### 1a) Production of Intermediates

### Intermediate 1.1:

### 4-Isothiocyanato-2-(trifluoromethyl)benzonitrile

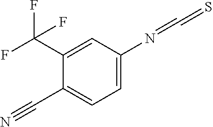

Thiophosgene (6.3 ml; 82.7 mmol) was slowly added to a solution of 4-amino-2-(trifluoromethyl)benzonitrile (14.0 g; 75.2 mmol) in tetrahydrofuran (140.0 ml) cooling in a water bath at room temperature. The reaction was stirred for 2 hours at room temperature and finally concentrated by evaporation. The residue was taken up in ethyl acetate and washed with a saturated solution of sodium chloride in water. The organic phase was filtered using a Whatman filter and concentrated by evaporation. The crude product was finally purified by chromatography (hexane-hexane/ethyl acetate 2:1) to yield the desired product (16.6 g; 72.7 mmol).

¹H-NMR (CDCl₃): 7.84 (m, 1H), 7.59 (m, 1H), 7.48 (m, 1H).

4-Isothiocyanato-2-(trifluoromethyl)benzonitrile is commercially available (e.g. Fluorochem, Oakwood, UK), too.

### Intermediate 1.2:

### 6-(1H-Imidazol-1-yl)pyridine-3-carbonitrile

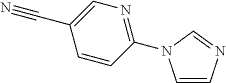

Potassium carbonate (4.99 g; 36.1 mmol) was added under stirring to a solution of 6-chloropyridine-3-carbonitrile (5.00 g; 36.1 mmol) and 1H-imidazole (2.46 g; 36.1 mmol) in dimethylsulfoxide (35.0 ml) at room temperature. The reaction mixture was stirred at 100° C. for 4.5 hours before 1H-imidazole (0.49 g; 7.2 mmol) was added again. The mixture was stirred an additional hour at 100° C. After cooling the reaction mixture was added to ice water. The precipitate was washed with cool water and dried under vacuum at 50° C. to yield the desired product (4.45 g; 26.2 mmol).

¹H-NMR (CDCl₃): 8.76 (m, 1H), 8.42 (m, 1H), 8.09 (m, 1H), 7.66 (m, 1H), 7.47 (m, 1H), 7.24 (m, 1H).

### Intermediate 1.3:

### 1-[6-(1H-Imidazol-1-yl)pyridin-3-yl]methanamine

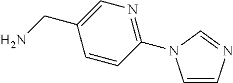

A solution of 6-(1H-imidazol-1-yl)pyridine-3-carbonitrile (4.45 g; 26.2 mmol) in a 7 N solution of ammonia in methanol (100 ml) was hydrogenated in an autoclave at 25° C. with the use of Raney Nickel (4.5 g; 50%) under a hydrogen atmosphere of 20 bar for 4 hours. The batch was filtered and concentrated by evaporation to yield the crude product (4.60 g) that was used without further purification.

### 1b) Production of End Product

6-(1H-Imidazol-1-yl)pyridine-3-methanamine (4.55 g; 26.1 mmol) was suspended in tetrahydrofuran (80.0 ml). After the addition of acetone cyanohydrin (8.0 ml; 87.2 mmol, Fluka), N,N-dimethylformamide (6.0 ml) and molecular sieves (4 Å) the reaction was stirred over night at room temperature. The reaction was filtered and concentrated by evaporation.

The residue was taken up in tetrahydrofuran (100.0 ml). 4-Isothiocyanato-2-(trifluoromethyl)benzonitrile (5.41 g; 23.7 mmol) and triethylamine (6.6 ml; 47.5 mmol) were added and the reaction was refluxed for 1 hour before it was concentrated by evaporation.

The residue was taken up in methanol (68.0 ml). A 4 N solution of hydrogen chloride in methanol (23.7 ml) was added and the reaction was stirred over night at room temperature. The reaction was diluted with ethyl acetate and washed with saturated solutions of sodium bicarbonate and sodium chloride. The organic phase was filtered using a Whatman filter and concentrated by evaporation. The residue was purified by column chromatography (dichloromethane/ethanol 95:5) to yield the desired product (3.03 g; 6.4 mmol).

¹H-NMR (CDCl₃): 8.54 (m, 1H), 8.36 (m, 1H), 8.04 (m, 1H), 7.98 (m, 1H), 7.92 (m, 1H), 7.80 (m, 1H), 7.64 (m, 1H), 7.38 (m, 1H), 7.21 (m, 1H), 5.13 (s, 2H), 1.55 (s, 6H).

### EXAMPLE 2

### 4-(4,4-Dimethyl-5-oxo-2-thioxo-3-{[6-(trifluoromethyl)pyridin-3-yl]methyl}imidazolidin-1-yl)-2-(trifluoromethyl)benzonitrile

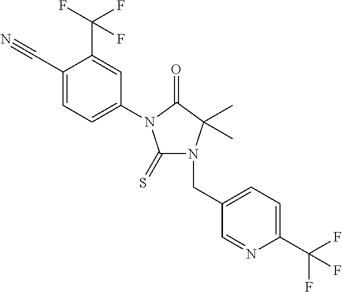

Example 2 was prepared using similar conditions as described in the preparation of Example 1. The required starting material 6-(trifluoromethyl)pyridine-3-methanamine was purchased from Apollo Scientific Limited, UK.

¹H-NMR (CDCl₃): 8.79 (m, 1H), 8.04 (m, 2H), 7.91 (m, 1H), 7.80 (m, 1H), 7.71 (m, 1H), 5.16 (s, 2H), 1.54 (s, 6H).

### EXAMPLE 3

### 4-[4,4-Dimethyl-3-({6-[2-(morpholin-4-yl)ethoxy]pyridin-3-yl}methyl)-5-oxo-2-thioxoimidazolidin-1-yl]-2-(trifluoromethyl)benzonitrile

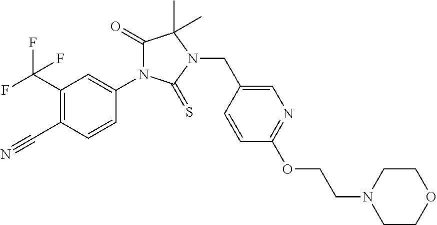

### 3a) Production of Intermediates

### Intermediate 3.1:

### 6-[2-(4-Morpholinyl)ethoxy]-3-pyridinecarbonitrile

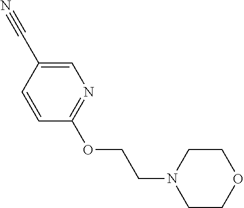

Under argon atmosphere, sodium hydride (60%; 0.65 g; 16.2 mmol) was added to a solution of 4-morpholineethanol (1.42 g; 11.0 mmol) in N,N-dimethylformamide (90 ml) at room temperature. The batch was stirred for 10 minutes at room temperature and then for 1 hour at 60° C. After cooling to room temperature, a solution of 6-chloropyridine-3-carbonitrile (1.50 g; 11.0 mmol) in N,N-dimethylformamide (10 ml) was added and the batch was stirred over night. Saturated sodium bicarbonate solution was added and the batch was extracted with chloroform. The organic phase was filtered using a Whatman filter and concentrated by evaporation. The residue was purified by column chromatography (hexane/ethyl acetate 2:3) to yield the desired product (1.48 g; 6.4 mmol).

### 3b) Production of End Product

Starting from 6-[2-(4-morpholinyl)ethoxy]-3-pyridinecarbonitrile, Example 3 was prepared using similar conditions as described in the preparation of Example 1.

¹H-NMR (DMSO-d₆): 8.34 (m, 1H), 8.27 (m, 2H), 8.04 (m, 1H), 7.79 (m, 1H), 6.77 (m, 1H), 5.01 (s, 2H), 4.32 (tr, 2H), 3.52 (br, 4H), 2.63 (br, 2H), 2.40 (br, 4H), 1.43 (s, 6H).

### EXAMPLE 4

### 4-(4,4-Dimethyl-3-{[6-(morpholin-4-yl)pyridin-3-yl]methyl}-5-oxo-2-thioxoimidazolidin-1-yl)-2-(trifluoromethyl)benzonitrile

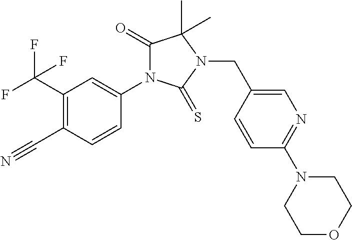

### 4a) Production of Intermediates

### Intermediate 4.1:

### 6-(Morpholin-4-yl)pyridine-3-carbonitrile

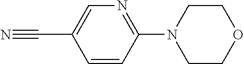

Diisopropylethylamine (12.4 ml; 72.5 mmol) and morpholine (7.2 ml; 82.9 mmol) were added dropwise to a solution of 6-chloropyridine-3-carbonitrile (10.1 g; 73.0 mmol) in N,N-dimethylformamide (78.0 ml) and water (26.0 ml) at room temperature. The batch was stirred at 90° C. over night. After cooling to room temperature, diluted solutions of sodium chloride and sodium bicarbonate were added and the batch was extracted with ethyl acetate (2×). The combined organic phases were filtered using a Whatman filter and concentrated by evaporation to give the crude product (13.6 g) that was used without further purification.

¹H-NMR (CDCl₃): 8.41 (m, 1H), 7.62 (m, 1H), 6.58 (m, 1H), 3.80 (m, 4H), 3.65 (m, 4H).

### 4b) Production of End Product

Starting from 6-(morpholin-4-yl)pyridine-3-carbonitrile, Example 4 was prepared using similar conditions as described in the preparation of Example 1.

¹H-NMR (CDCl₃): 8.24 (m, 1H), 7.96 (m, 1H), 7.90 (m, 1H), 7.78 (m, 1H), 7.72 (m, 1H), 6.63 (m, 1H), 5.01 (s, 2H), 3.82 (tr, 4H), 3.51 (tr, 4H), 1.50 (s, 6H).

### EXAMPLE 5

### 4-(4,4-Dimethyl-5-oxo-3-{[6-(tetrahydro-2H-pyran-4-yloxy)pyridin-3-yl]methyl}-2-thioxoimidazolidin-1-yl)-2-(trifluoromethyl)benzonitrile

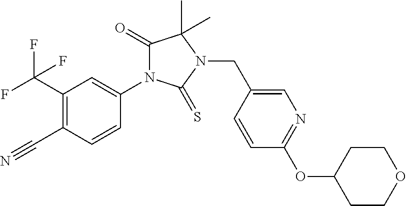

Example 5 was prepared using similar conditions as described in the preparation of Example 1. The required starting material 6-(tetrahydro-2H-pyran-4-yloxy)pyridine-3-carbonitrile was purchased from ABCR GmbH & Co. KG, Germany.

¹H-NMR (DMSO-d₆): 8.34 (m, 1H), 8.28 (m, 1H), 8.24 (m, 1H), 8.04 (m, 1H), 7.79 (m, 1H), 6.75 (m, 1H), 5.11 (m, 1H), 5.00 (s, 2H), 3.81 (m, 2H), 3.43 (m, 2H), 1.94 (m, 2H), 1.57 (m, 2H), 1.43 (s, 6H).

### EXAMPLE 6

### 4-(3-{[4-Amino-2-(morpholin-4-yl)pyrimidin-5-yl]methyl}-4,4-dimethyl-5-oxo-2-thioxoimidazolidin-1-yl)-2-(trifluoromethyl)benzonitrile

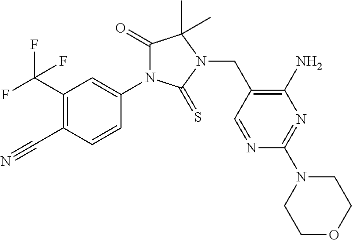

### 6a) Production of Intermediates

### Intermediate 6.1:

### 4-Amino-2-(4-morpholinyl)-5-pyrimidinecarbonitrile

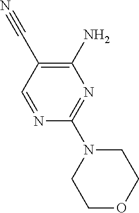

A solution of 4-amino-2-chloro-5-pyrimidinecarbonitrile (3.0 g; 19.0 mmol) and morpholine (2.0 ml, 23.3 mmol) in N,N-dimethylformamide (30.0 ml) was stirred at 60° C. After 20 hours further morpholine (1.0 ml; 11.6 mmol) was added and the batch was stirred for additional 5 hours. The batch was concentrated by evaporation. The residue was taken up in ethyl acetate and washed with 10% citric acid solution, saturated sodium bicarbonate solution and finally saturated sodium chloride solution. The organic phase was dried over sodium sulfate, filtered and concentrated by evaporation to give the desired product (3.5 g) that was used without further purification.

¹H-NMR (DMSO-dₒ): 8.24 (s, 1H), 7.27 (br, 2H), 3.67 (m, 4H), 3.56 (m, 4H).

### 6b) Production of End Product

Starting from 4-amino-2-(4-morpholinyl)-5-pyrimidinecarbonitrile, Example 6 was prepared using similar conditions as described in the preparation of Example 1.

¹H-NMR (CDCl₃): 7.97 (m, 1H), 7.90 (m, 1H), 7.86 (m, 1H), 7.75 (m, 1H), 5.55 (br, 2H), 5.05 (s, 2H), 3.74 (m, 8H), 1.53 (s, 6H).

### EXAMPLE 7

### 4-(4,4-Dimethyl-3-{[6-(2-methylmorpholin-4-yl)pyridin-3-yl]methyl}-5-oxo-2-thioxoimidazolidin-1-yl)-2-(trifluoromethyl)benzonitrile

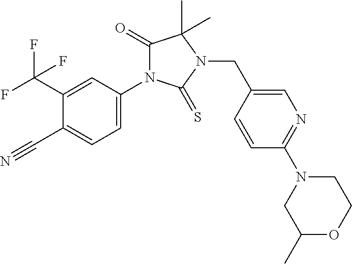

Example 7 was prepared using similar conditions as described in the preparation of Example 1. The required starting material 6-(2-methylmorpholin-4-yl)pyridine-3-methanamine was purchased from Ukrorgsyn-BB, China.

¹H-NMR (CDCl₃): 8.22 (m, 1H), 7.97 (m, 1H), 7.91 (m, 1H), 7.79 (m, 1H), 7.71 (m, 1H), 6.62 (m, 1H), 5.01 (s, 2H), 4.02 (m, 3H), 3.70 (m, 2H), 2.96 (m, 1H), 2.62 (m, 11-1), 1.50 (s, 6H), 1.26 (d, 3H).

### EXAMPLE 8

### 4-{3-[(6-Methoxypyridin-3-yl)methyl]-4,4-dimethyl-5-oxo-2-thioxoimidazolidin-1-yl}-2-(trifluoromethyl)benzonitrile

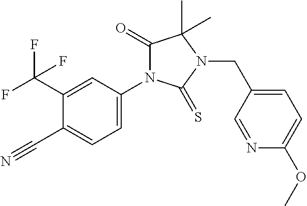

Example 8 was prepared using similar conditions as described in the preparation of Example 1. The required starting material 6-methoxypyridine-3-methanamine was purchased from Enamine Ltd., Ukraine.

¹H-NMR (CDCl₃): 8.21 (m, 1H), 7.97 (m, 1H), 7.91 (m, 1H), 7.80 (m, 1H), 7.77 (m, 1H), 6.76 (m, 1H), 5.05 (s, 2H), 3.94 (s, 3H), 1.49 (s, 6H).

### EXAMPLE 9

### 4-(3-{[6-(1-Imino-1-oxido-1λ⁶-thiomorpholin-4-yl)pyridin-3-yl]methyl}-4,4-dimethyl-5-oxo-2-thioxoimidazolidin-1-yl)-2-(trifluoromethyl)benzonitrile

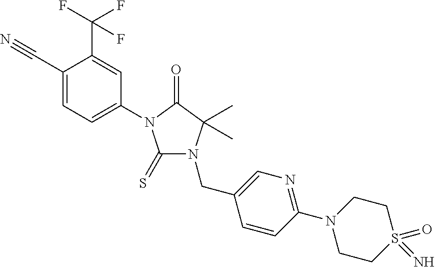

### 9a) Production of Intermediates

### Intermediate 9.1:

### 6-(Thiomorpholin-4-yl)pyridine-3-carbonitrile

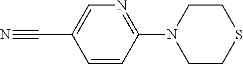

A solution of thiomorpholine (1.34 g; 13.0 mmol) in N,N-dimethylformamide (2.0 ml) was added dropwise to a solution of 6-chloropyridine-3-carbonitrile (1.00 g; 7.2 mmol) in N,N-dimethylformamide (8.0 ml) at room temperature. The batch was stirred at room temperature over night. Cold water (50.0 ml) was added and the batch was filtered. The precipitate was washed with water/ethanol (4:1) and finally dried in vacuo to give the desired product (1.08 g; 5.3 mmol).

¹H-NMR (DMSO-d₆): 8.45 (m, 1H), 7.81 (m, 1H), 6.91 (m, 1H), 3.97 (m, 4H), 2.58 (m, 4H).

### Intermediate 9.2:

### 6-(1-oxidothiomorpholin-4-yl)pyridine-3-carbonitrile

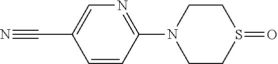

Iron(III)chloride (12 mg; 0.07 mmol) was added to a solution of 6-(thiomorpholin-4-yl)pyridine-3-carbonitrile (500 mg; 2.4 mmol) in acetonitrile (1.8 ml) and the batch was stirred for 10 minutes at room temperature. Periodic acid (500 mg; 2.6 mmol) was added and the batch was stirred for 2.5 hours at room temperature. The batch was diluted with ethyl acetate and washed with a saturated solution of sodium chloride. The organic phase was filtered using a Whatman filter and concentrated in vacuo. The residue was purified by column chromatography (dichloromethane/ethanol 9:1) to give the desired product (248 mg; 1.0 mmol).

¹H-NMR (DMSO-d₆): 8.50 (m, 1H), 7.87 (m, 1H), 7.06 (m, 1H), 4.27 (m, 2H), 3.89 (m, 2H), 2.84 (m, 2H), 2.68 (m, 2H).

### Intermediate 9.3:

### N-[4-(5-Cyanopyridin-2-yl)-1-oxido-1λ⁶-thiomorpholin-1-ylidene]-2,2,2-trifluoroacetamide

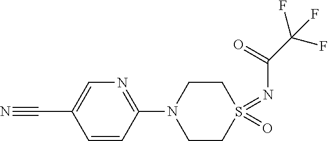

To a suspension of 6-(1-oxidothiomorpholin-4-yl)pyridine-3-carbonitrile (187 mg; 0.85 mmol), trifluoroacetamide (191 mg; 1.69 mmol), magnesium oxide (136 mg; 3.38 mmol), and rhodium(II) acetate dimer (17 mg; 0.09 mmol) in dichloromethane (20 ml) was added iodobenzene diacetate (408 mg; 1.27 mmol) at 40° C. The resulting mixture was stirred 6 hours at 40° C. and finally concentrated on silica. The residue was purified by column chromatography (dichloromethane/ethanol 97:3) to give the desired product (266 mg, 0.80 mmol).

¹H-NMR (DMSO-d₆): 8.56 (m, 1H), 7.97 (m, 1H), 7.13 (m, 1H), 4.46 (m, 2H), 3.89 (m, 4H), 3.81 (m, 2H).

### Intermediate 9.4:

### 6-(1-Imino-1-oxido-1λ⁶-thiomorpholin-4-yl)pyridine-3-carbonitrile

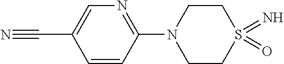

To a solution of N-[4-(5-cyanopyridin-2-yl)-1-oxido-1λ⁶-thiomorpholin-1-ylidene]-2,2,2-trifluoroacetamide (248 mg; 0.75 mmol) in methanol (16.0 ml) at room temperature was added potassium carbonate (516 mg; 3.71 mmol). The mixture was stirred for 1 hour at room temperature. The batch was diluted with ethyl acetate and washed with a saturated solution of sodium chloride. The organic phase was filtered using a Whatman filter and concentrated in vacuo. The residue was purified by column chromatography (dichloromethane/ethanol 95:5) to give the desired product (104 mg; 0.44 mmol).

¹H-NMR (CDCl₃): 8.48 (m, 1H), 7.72 (m, 1H), 6.74 (m, 1H), 4.29 (m, 2H), 4.17 (m, 2H), 3.11 (m, 4H), 2.66 (br, 1H).

### Intermediate 9.5:

### Ethyl [4-(5-cyanopyridin-2-yl)-1-oxido-1λ⁶-thiomorpholin-1-ylidene]carbamate

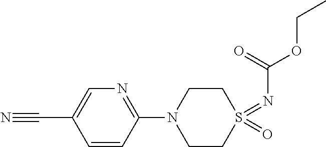

To a solution of 6-(1-imino-1-oxido-1λ⁶-thiomorpholin-4-yl)pyridine-3-carbonitrile (100 mg; 0.42 mmol) in pyridine (4.0 ml) at 0° C. was added ethyl chloroformate (60 mg; 0.55 mmol). The mixture was slowly warmed to room temperature and stirred over night. The batch was concentrated in vacuo and the residue was taken up in ethyl acetate. The organic phase was washed with a saturated solution of sodium chloride, filtered using a Whatman filter and concentrated in vacuo. The residue was purified by column chromatography (dichloromethane/ethanol 95:5) to give the desired product (91 mg; 0.29 mmol).

¹H-NMR (CDCl₃): 8.49 (m, 1H), 7.76 (m, 1H), 6.78 (m, 1H), 4.43 (m, 2H), 4.16 (q, 2H), 4.08 (m, 2H), 3.69 (m, 2H), 3.30 (m, 2H), 1.31 (tr, 3H).

### Intermediate 9.6:

### Ethyl {4-[5-({3-[4-cyano-3-(trifluoromethyl)phenyl]-5,5-dimethyl-4-oxo-2-thioxoimidazolidin-1-yl}methyl)pyridin-2-yl]-1-oxido-1λ⁶-thiomorpholin-1-ylidene}carbamate

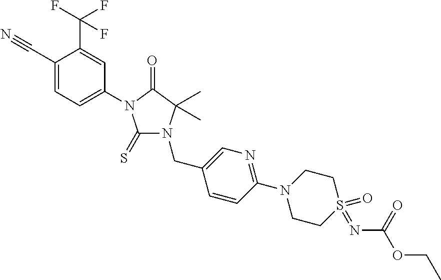

Starting from ethyl [4-(5-cyanopyridin-2-yl)-1-oxido-1λ⁶-thiomorpholin-1-ylidene]carbamate, intermediate 9.6 was prepared using similar conditions as described in the preparation of Example 1.

¹H-NMR (CDCl₃): 8.28 (m, 1H), 7.97 (m, 1H), 7.91 (m, 1H), 7.80 (m, 2H), 6.76 (m, 1H), 5.01 (s, 2H), 4.36 (m, 2H), 4.16 (q, 2H), 4.00 (m, 2H), 3.67 (m, 2H), 3.29 (m, 2H), 1.53 (s, 6H), 1.30 (tr, 3H).

### 9b) Production of End Product

Ethyl {-4-[5-({3-[4-cyano-3-(trifluoromethyl)phenyl]-5,5-dimethyl-4-oxo-2-thioxoimidazolidin-1-yl}methyl)pyridin-2-yl]-1-oxido-1λ⁶-thiomorpholin-1-ylidene}carbamate (29 mg; 0.048 mmol) was dissolved under stirring in concentrated sulfuric acid (0.54 ml) at room temperature. After 25 hours the batch was cautiously added to ice water and basified with saturated sodium bicarbonate solution. The batch was extracted with ethyl acetate (2×). The combined organic phases were filtered using a Whatman filter and finally concentrated in vacuo. The residue was purified by HPLC chromatography to give the desired product (12 mg; 0.02 mmol).
System: Waters Autopurificationsystem: Pump 254, Sample Manager 2767, CFO, DAD 2996, ELSD 2424, SQD 3001
Column: Kromasil C18 5 μm 150×21.2 mm
Solvent: A=H₂O+0.1% HCOOH
   B=Acetonitrile
Gradient: 0-1 min 10% B, 1-7.5 min 10-100% B, 7.5-10 min 100% B
Flow: 25 mL/min
Temperature: Room temperature
Detection: DAD scan range 210-400 nm
   MS ESI+, ESI−, scan range 160-1000 m/z
   ELSD
Retention 6.4-6.8 minutes

¹H-NMR (CDCl₃): 8.26 (m, 1H), 7.97 (m, 1H), 7.91 (m, 1H), 7.78 (m, 2H), 6.73 (m, 1H), 5.01 (s, 2H), 4.21 (m, 2H), 4.09 (m, 2H), 3.10 (tr, 4H), 2.60 (br, 1H), 1.52 (s, 6H).

### EXAMPLE 10

### 4-(3-{[6-(2-Hydroxy-2-methylpropoxy)pyridin-3-yl]methyl}-4,4-dimethyl-5-oxo-2-thioxoimidazolidin-1-yl)-2-(trifluoromethyl)benzonitrile

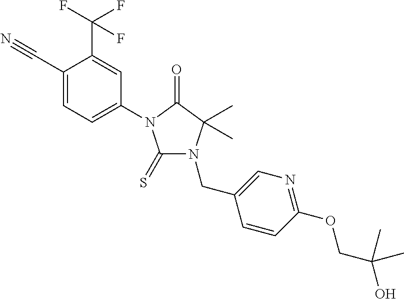

### 10a) Production of Intermediates

### Intermediate 10.1:

### 6-(2-Hydroxy-2-methylpropoxy)pyridine-3-carbonitrile

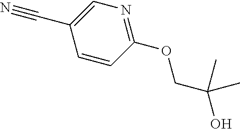

Sodium hydride (60%; 346 mg) was added to a solution of 2-methylpropane-1,2-diol (650 mg; 7.2 mmol) in N,N-dimethylformamide (66.7 ml) and the batch was stirred for 1 hour at room temperature. A solution of 6-chloropyridine-3-carbonitrile (1000 mg) in N,N-dimethylformamide (6.7 ml) was added and the batch was stirred over night at room temperature. The mixture was diluted with ice and a diluted solution of sodium chloride and extracted with ethyl acetate (3×). The combined organic phases were washed with a diluted sodium chloride solution, dried over sodium sulfate and filtered. The filtrate was concentrated in vacuo and the residue was purified by column chromatography (hexane→hexane/ethyl acetate 1:1) to give the desired product (508 mg; 2.6 mmol).

¹H-NMR (CDCl₃): 8.46 (m, 1H), 7.81 (m, 1H), 6.88 (m, 1H), 4.26 (s, 2H), 2.35 (br, 1H), 1.33 (s, 6H).

### 10b) Production of End Product

Starting from 6-(2-hydroxy-2-methylpropoxy)pyridine-3-carbonitrile, Example 10 was prepared using similar conditions as described in the preparation of Example 1.

¹H-NMR (CDCl₃): 8.17 (m, 1H), 7.97 (m, 1H), 7.91 (m, 1H), 7.81 (m, 2H), 6.82 (m, 1H), 5.05 (s, 2H), 4.21 (s, 2H), 3.08 (br, 1H), 1.50 (s, 6H), 1.33 (s, 6H).

### EXAMPLE 11

### 4-(3-{[6-(2-Methoxyethoxy)pyridin-3-yl]methyl}-4,4-dimethyl-5-oxo-2-thioxoimidazolidin-1-yl)-2-(trifluoromethyl)benzonitrile

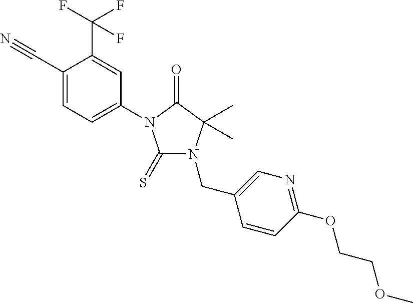

### 11a) Production of Intermediates

### Intermediate 11.1:

### 4-{3-[(6-Chloropyridin-3-yl)methyl]-4,4-dimethyl-5-oxo-2-thioxoimidazolidin-1-yl}-2-(trifluoromethyl)benzonitrile

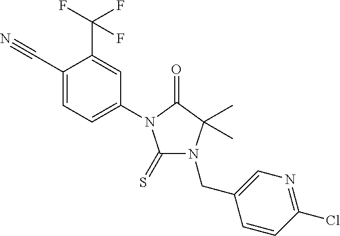

Starting from 6-chloropyridine-3-methanamine (Aldrich), Intermediate 11.1 was prepared using similar conditions as described in the preparation of Example 1.

¹H-NMR (CDCl₃): 8.45 (m, 1H), 7.98 (m, 1H), 7.91 (m, 1H), 7.87 (m, 1H), 7.79 (m, 1H), 7.35 (m, 1H), 5.08 (s, 2H), 1.51 (s, 6H).

### 11b) Production of End Product

A 1 N solution of potassium tert-butylate in tetrahydrofuran (0.18 ml; 0.18 mmol) was added under argon to a solution of 2-methoxy-ethanol (11 mg; 0.15 mmol) in tetrahydrofuran (0.6 ml) at room temperature. The batch was stirred for 30 minutes at 50° C. before a solution of 4-{3-[(6-chloropyridin-3-yl)methyl]-4,4-dimethyl-5-oxo-2-thioxoimidazolidin-1-yl}-2-(trifluoromethyl)benzonitrile (80 mg; 0.18 mmol) in tetrahydrofuran (0.2 ml) was added. After 19 hours at reflux further 2-methoxy-ethanol (11 mg; 0.15 mmol) and 1 N solution of potassium tert-butylate in tetrahydrofuran (0.18 ml; 0.18 mmol) were added and the batch was refluxed for additional 28 hours. Further 2-methoxy-ethanol (55 mg; 0.75 mmol) was added and the batch was refluxed for 2 days. Finally, further 1 N solution of potassium tert-butylate in tetrahydrofuran (0.44 ml; 0.44 mmol) was added and the batch was refluxed for 2 days. After cooling, the batch was diluted with water and extracted with dichloromethane (2×). The combined organic phases were filtered using a Whatman filter and concentrated by evaporation. The residue was purified by HPLC to yield the desired product (5 mg; 0.01 mmol).
System: Waters Autopurification
Column: XBridge C18 5μ 100×30 mm
Solvent A: H₂O/0.1% HCOOH
Solvent B: Acetonitrile
Gradient:

| | | |
|---|---|---|
| 0 min | 99% A | 1% B |
| 1.00 min | 99% A | 1% B |
| 7.50 min | 1% A | 99% B |
| 10.00 min | 1% A | 99% B |

Flow: 50.0 mL/min
Detector: DAD scan range 210-400 nm
   MS ESI+, ESI−, scan range 160-1000 m/z

¹H-NMR (CDCl₃): 8.18 (m, 1H), 7.97 (m, 1H), 7.91 (m, 1H), 7.77 (m, 2H), 6.82 (m, 1H), 5.05 (s, 2H), 4.47 (m, 2H), 3.75 (m, 2H), 3.44 (s, 3H), 1.48 (s, 6H).

### EXAMPLE 12

### 4-(4,4-Dimethyl-3-{[6-(4-methyl-1,4-diazepan-1-yl)pyridin-3-yl]methyl}-5-oxo-2-thioxoimidazolidin-1-yl)-2-(trifluoromethyl)benzonitrile

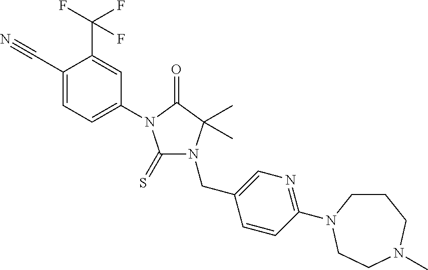

### Production of End Product

Example 12 was prepared using similar conditions as described in the preparation of Example 1. The required starting material 6-(4-methyl-1,4-diazepan-1-yl)pyridine-3-methanamine was purchased from Ukrorgsyn (for details see above).

¹H-NMR (CDCl₃): 8.17 (m, 1H), 7.95 (m, 2H), 7.78 (m, 1H), 7.64 (m, 1H), 6.46 (m, 1H), 4.98 (s, 2H), 3.61 (m, 2H), 3.81 (tr, 2H), 2.69 (m, 2H), 2.57 (m, 2H), 2.38 (s, 3H), 2.01 (m, 2H), 1.50 (s, 6H).

### EXAMPLE 13

### 4-(4,4-Dimethyl-3-{[2-methyl-6-(trifluoromethyl)pyridin-3-yl]methyl}-5-oxo-2-thioxoimidazolidin-1-yl)-2-(trifluoromethyl)benzonitrile

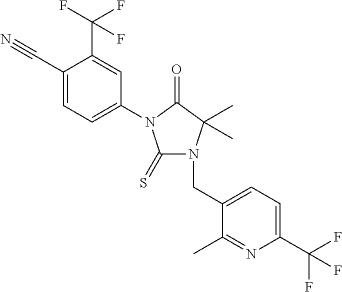

Example 13 was prepared using similar conditions as described in the preparation of Example 1. The required starting material 2-methyl-6-(trifluoromethyl)pyridine-3-carbonitrile was purchased from Fluorochem, Oakwood, UK

¹H-NMR (CDCl₃): 7.99 (m, 1H), 7.95 (m, 1H), 7.84 (m, 1H), 7.70 (m, 1H), 7.54 (m, 1H), 5.08 (s, 2H), 2.72 (s, 3H), 1.53 (s, 6H).

### EXAMPLE 14

### 4-[3-({6-[4-(Hydroxymethyl)piperidin-1-yl]pyridin-3-yl}methyl)-4,4-dimethyl-5-oxo-2-thioxoimidazolidin-1-yl]-2-(trifluoromethyl)benzonitrile

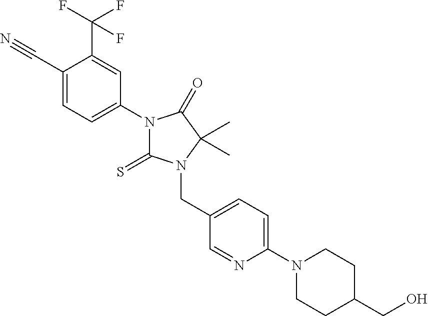

Example 14 was prepared using similar conditions as described in the preparation of Example 1. The required starting material 1-[5-(aminomethyl)pyridin-2-yl]piperidine-4-methanol was purchased from Ukrorgsyn (for details see above).

¹H-NMR (CDCl₃): 8.20 (m, 1H), 7.93 (m, 2H), 7.78 (m, 1H), 7.65 (m, 1H), 6.65 (m, 1H), 4.98 (s, 2H), 4.30 (m, 2H), 3.53 (m, 2H), 2.87 (m, 2H), 2.70 (br, 1H), 1.78 (m, 3H), 1.49 (s, 6H), 1.28 (m, 2H).

### EXAMPLE 15

### 4-(4,4-Dimethyl-3-{[6-(2-methyl-1H-imidazol-1-yl)pyridin-3-yl]methyl}-5-oxo-2-thioxoimidazolidin-1-yl)-2-(trifluoromethyl)benzonitrile

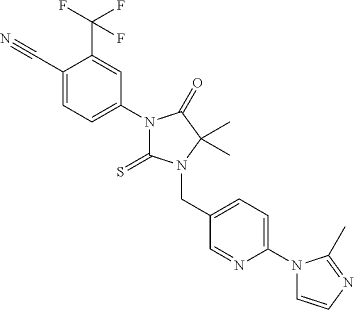

Example 15 was prepared using similar conditions as described in the preparation of Example 1. The required starting material 6-(2-methyl-1H-imidazol-1-yl)pyridine-3-methanamine was purchased from Ukrorgsyn (for details see above).

¹H-NMR (CDCl₃): 8.60 (m, 1H), 8.04 (m, 1H), 7.99 (m, 1H), 7.93 (m, 1H), 7.81 (m, 1H), 7.35 (m, 1H), 7.30 (m, 1H), 7.05 (m, 1H), 5.15 (s, 2H), 2.63 (s, 3H), 1.56 (s, 6H).

### EXAMPLE 16

### 4-(3-{[6-(4,4-Dimethyl-2-oxopyrrolidin-1-yl)pyridin-3-yl]methyl}-4,4-dimethyl-5-oxo-2-thioxoimidazolidin-1-yl)-2-(trifluoromethyl)benzonitrile

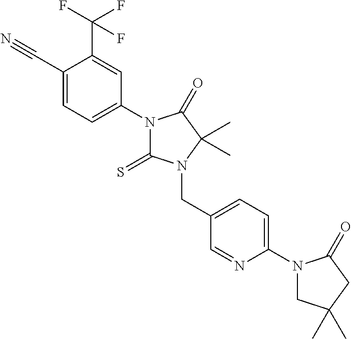

### 16a) Production of Intermediates

### Intermediate 16.1:

### 6-(4,4-Dimethyl-2-oxopyrrolidin-1-yl)pyridine-3-carbonitrile

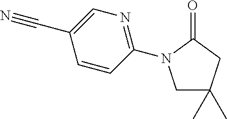

A solution of 4,4-dimethylpyrrolidin-2-one (purchased from Key Organics Limited, UK; 991 mg, 8.76 mmol) in toluene (2 ml) was added to a suspension of sodium hydride (60%, 192 mg) in toluene (1 ml) at 0° C. The batch was warmed to room temperature before a suspension of 6-chloropyridine-3-carbonitrile (606 mg; 4.4 mmol) in toluene (2 ml) was added. The batch was stirred at 95° C. for 5 hours. After cooling, the batch was added to ice water and extracted with ethyl acetate (2×). The combined organic phases were filtered using a Whatman filter and concentrated by evaporation. The residue was purified by column chromatography (hexane/ethyl acetate 1:1) to give the desired product (383 mg, 1.78 mmol).

¹H-NMR (CDCl₃): 8.59 (m, 2H), 7.90 (m, 1H), 3.83 (s, 2H), 2.52 (s, 2H), 1.24 (s, 6H).

### b) Production of End Product

Starting from 6-(4,4-dimethyl-2-oxopyrrolidin-1-yl)pyridine-3-carbonitrile, Example 16 was prepared using similar conditions as described in the preparation of Example 1.

¹H-NMR (CDCl₃): 8.43 (m, 1H), 8.41 (m, 1H), 7.97 (m, 1H), 7.91 (m, 1H), 7.82 (m, 2H), 5.08 (s, 2H), 3.82 (s, 2H), 2.49 (s, 2H), 1.50 (s, 6H), 1.24 (s, 6H).

### EXAMPLE 17

### 4-(3-{[2-(1H-Imidazol-1-yl)pyrimidin-5-yl]methyl}-4,4-dimethyl-5-oxo-2-thioxoimidazolidin-1-yl)-2-(trifluoromethyl)benzonitrile

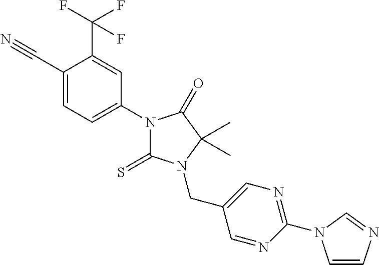

Starting from 2-(1H-imidazol-1-yl)pyrimidine-5-methanamine hydrochloride which was purchased from Anichem Inc, North Brunswick, USA Example 17 was prepared using similar conditions as described in the preparation of Example 1.

¹H-NMR (CDCl₃): 8.85 (m, 2H), 8.61 (m, 1H), 7.99 (m, 1H), 7.90 (m, 1H), 7.87 (m, 1H), 7.78 (m, 1H), 7.18 (m, 1H). 5.06 (s, 2H), 1.61 (s, 6H).

### EXAMPLE 18

### 4-(4,4-Dimethyl-3-{[6-(1-methyl-1H-pyrazol-4-yl)pyridin-3-yl]methyl}-5-oxo-2-thioxoimidazolidin-1-yl)-2-(trifluoromethyl)benzonitrile

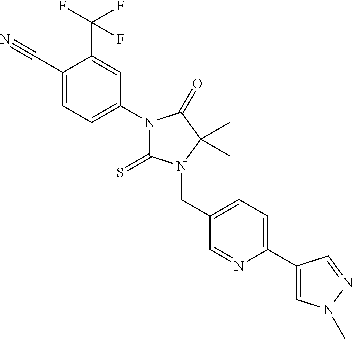

### 18a) Production of Intermediates

### Intermediate 18.1:

### 6-(1-Methyl-1H-pyrazol-4-yl)pyridine-3-carbonitrile

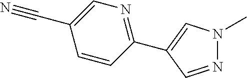

Under argon a solution of sodium carbonate (348 mg, 3.28 mmol) in water (1.5 ml) was added to a solution of 6-chloropyridine-3-carbonitrile (200 mg, 1.44 mmol) and 1-methyl-4-(4,4,5,5-tetramethyl-1,3,2-dioxaborolan-2-yl)-1H-pyrazole (274 mg, 1.32 mmol) in 1,2-dimethoxyethan (2.9 ml). Finally, tetrakis(triphenylphosphine)palladium(0) (166 mg, 0.14 mmol) was added and the batch was stirred in a sealed tube in a microwave at 135° C. for 30 min. After cooling, the batch was diluted with ethyl acetate and washed with diluted solutions of 0.5 N sodium hydroxide and sodium chloride. The organic phase was dried over sodium sulfate, filtered and concentrated. The residue was purified by column chromatography (hexane 4 ethyl acetate) to give the desired product (190 mg; 1.03 mmol).

¹H-NMR (D6-DMSO): 8.87 (m, 1H), 8.40 (m, 1H), 8.19 (m, 1H), 8.08 (m, 1H), 7.81 (m, 1H), 3.86 (s, 3H).

### b) Production of End Product

Starting from 6-(1-methyl-1H-pyrazol-4-yl)pyridine-3-carbonitrile Example 18 was prepared using similar conditions as described in the preparation of Example 1.

¹H-NMR (CDCl₃): 8.59 (m, 1H), 7.97 (m, 1H), 7.94 (m, 1H), 7.92 (m, 2H), 7.82 (m, 2H), 7.45 (m, 1H), 5.11 (s, 2H), 3.96 (s, 3H), 1.52 (s, 6H).

### EXAMPLE 19

### 4-(4,4-Dimethyl-3-{[6-(4-methyl-1H-imidazol-1-yl)pyridin-3-yl]methyl}-5-oxo-2-thioxoimidazolidin-1-yl)-2-(trifluoromethyl)benzonitrile

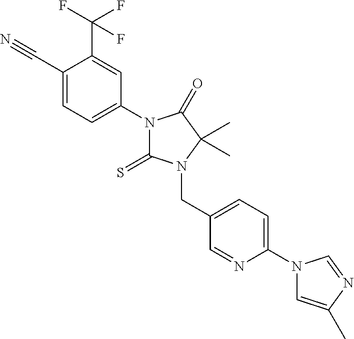

### 19a) Production of Intermediates

### Intermediate 19.1:

### 6-(4-Methyl-1H-imidazol-1-yl)pyridine-3-carbonitrile

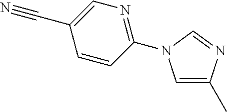

Potassium carbonate (2.00 g; 14.4 mmol) was added under stirring to a solution of 6-chloropyridine-3-carbonitrile (2.00 g; 14.4 mmol) and 4-methyl-1H-imidazole (1.42 g; 17.3 mmol) in dimethylsulfoxide (18.0 ml) at room temperature. The reaction mixture was stirred at 100° C. for 6 hours before further potassium carbonate (0.40 g; 2.9 mmol) and 4-methyl-1H-imidazole (0.24 g; 2.9 mmol) were added. The mixture was stirred additional 3 hours at 100° C. After cooling, the reaction mixture was added to ice water. The precipitate was washed with cool water and dried to yield the desired product (1.86 g; 10.1 mmol).

¹H-NMR (D6-DMSO): 8.95 (m, 1H), 8.54 (m, 1H), 8.49 (m, 1H), 7.96 (m, 1H), 7.74 (m, 1H), 2.19 (s, 3H).

### b) Production of End Product

Starting from 6-(4-methyl-1H-imidazol-1-yl)pyridine-3-carbonitrile, Example 19 was prepared using similar conditions as described in the preparation of Example 1.

¹H-NMR (CDCl₃): 8.51 (m, 1H), 8.35 (m, 1H), 8.00 (m, 2H), 7.92 (m, 1H), 7.80 (m, 1H), 7.34 (m, 2H), 5.12 (s, 2H), 2.32 (s, 3H), 1.54 (s, 6H).

### EXAMPLE 20

### 4-(4,4-Dimethyl-3-{[6-(1-methyl-1H-pyrazol-5-yl)pyridin-3-yl]methyl}-5-oxo-2-thioxoimidazolidin-1-yl)-2-(trifluoromethyl)benzonitrile

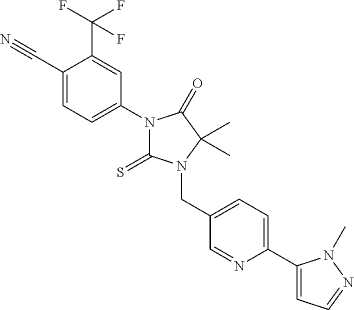

### 20a) Production of Intermediates

### Intermediate 20.1:

### 6-(1-Methyl-1H-pyrazol-5-yl)pyridine-3-carbonitrile

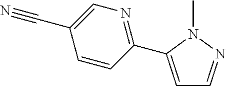

Under argon a solution of sodium carbonate (870 mg, 8.21 mmol) in water (3.6 ml) was added to a solution of 6-chloropyridine-3-carbonitrile (500 mg, 3.61 mmol) and 1-methyl-5-(4,4,5,5-tetramethyl-1,3,2-dioxaborolan-2-yl)-1H-pyrazole (685 mg, 3.30 mmol) in 1,2-dimethoxyethan (7.3 ml). Finally, tetrakis(triphenylphosphine)palladium(0) (417 mg, 0.36 mmol) was added and the batch was stirred in a sealed tube in a microwave at 135° C. for 30 min. After cooling, the batch was diluted with ethyl acetate and washed with diluted solutions of 0.5 N sodium hydroxide and sodium chloride. The organic phase was dried over sodium sulfate, filtered and concentrated. The residue was purified by column chromatography (hexane→ethyl acetate) to give the desired product (560 mg; 3.04 mmol).

¹H-NMR (D6-DMSO): 9.07 (m, 1H), 8.36 (m, 1H), 8.01 (m, 1H), 7.51 (m, 1H), 6.98 (m, 1H), 4.14 (s, 3H).

### b) Production of End Product

Starting from 6-(1-methyl-1H-pyrazol-5-yl)pyridine-3-carbonitrile, Example 20 was prepared using similar conditions as described in the preparation of Example 1.

¹H-NMR (CDCl₃): 8.72 (m, 1H), 7.99 (m, 1H), 7.91 (m, 2H), 7.81 (m, 1H), 7.61 (m, 1H), 7.51 (m, 1H), 6.61 (m, 1H). 5.15 (s, 2H), 4.25 (s, 3H), 1.54 (s, 6H).

### EXAMPLE 21

### 4-(3-{[6-(4-Chloro-2-methyl-1H-imidazol-1-yl)pyridin-3-yl]methyl}-4,4-dimethyl-5-oxo-2-thioxoimidazolidin-1-yl)-2-(trifluoromethyl)benzonitrile

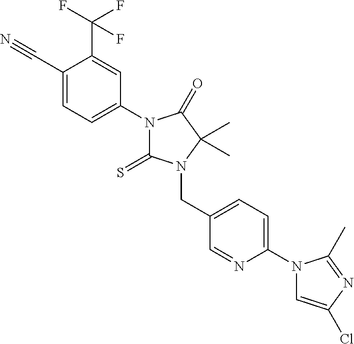

### 21a) Production of Intermediates

### Intermediate 21.1:

### 6-(4-Chloro-2-methyl-1H-imidazol-1-yl)pyridine-3-carbonitrile

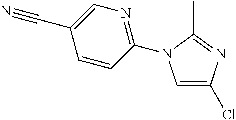

Potassium carbonate (734 mg; 5.3 mmol) was added under stirring to a solution of 6-chloropyridine-3-carbonitrile (735 mg; 5.3 mmol) and 5-chloro-2-methyl-1H-imidazole (619 mg; 5.3 mmol) in dimethylsulfoxide (5.2 ml) at room temperature. The reaction mixture was stirred at 100° C. for 2 hours. After cooling, the reaction mixture was added to ice water. The precipitate was washed with cool water and dried to yield the desired product (935 mg; 4.3 mmol).

¹H-NMR (DMSO-d₆): 9.03 (m, 1H), 8.54 (m, 1H), 7.89 (m, 1H), 7.84 (m, 1H), 2.56 (s, 3H).

### b) Production of End Product

Starting from 6-(4-chloro-2-methyl-1H-imidazol-1-yl)pyridine-3-carbonitrile, Example 21 was prepared using similar conditions as described in the preparation of Example 1.

¹H-NMR (CDCl₃): 8.61 (m, 1H), 8.07 (m, 1H), 7.99 (m, 1H), 7.93 (m, 1H), 7.81 (m, 1H), 7.33 (m, 1H), 7.21 (m, 1H), 5.15 (s, 2H), 2.60 (s, 3H), 1.57 (s, 6H).

### EXAMPLE 22

### 4-[4,4-Dimethyl-3-({6-[1-(methylimino)-1-oxido-1λ⁶-thiomorpholin-4-yl]pyridin-3-yl}methyl)-5-oxo-2-thioxoimidazolidin-1-yl]-2-(trifluoromethyl)benzonitrile

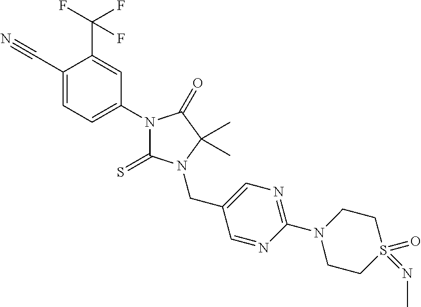

### 22a) Production of Intermediates

### Intermediate 22.1:

### 6-[1-(Methylimino)-1-oxido-1λ⁶-thiomorpholin-4-yl]pyridine-3-carbonitrile

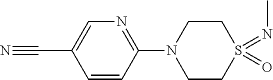

Formaldehyde (0.12 ml, 4.2 mmol) was added to a solution of 6-(1-imino-1-oxido-1λ⁶-thiomorpholin-4-yl)pyridine-3-carbonitrile (Intermediate 12.4) (200 mg, 0.85 mmol) in formic acid (4.30 ml) and the batch was stirred at 80° C. for 24 hours. After cooling, the batch was added to water and extracted with ethyl acetate (1×) and dichloromethane (3×). The combined organic phases were filtered using a Whatman filter and concentrated by evaporation. The residue was purified by column chromatography (dichloromethane/ethanol 95:5) to give the desired product (82 mg, 0.33 mmol).

¹H-NMR (DMSO-d₆): 8.51 (m, 1H), 7.92 (m, 1H), 7.06 (m, 1H), 4.26 (m, 2H), 3.76 (m, 2H), 3.14 (m, 2H), 3.02 (m, 2H), 2.63 (s, 3H).

### b) Production of End Product

Starting from 6-[1-(methylimino)-1-oxido-1λ⁶-thiomorpholin-4-yl]pyridine-3-carbonitrile, Example 22 was prepared using similar conditions as described in the preparation of Example 1.

¹H-NMR (CDCl₃): 8.25 (m, 1H), 7.97 (m, 1H), 7.91 (m, 1H), 7.77 (m, 2H), 6.72 (m, 1H), 5.00 (s, 2H), 4.21 (m, 2H). 4.01 (s, 2H), 3.01 (m, 4H), 2.85 (s, 3H), 1.52 (s, 6H).

### EXAMPLE 23

### 4-[4,4-Dimethyl-5-oxo-2-thioxo-3-({6-[4-(trifluoromethyl)-1H-imidazol-1-yl]pyridin-3-yl}methyl)imidazolidin-1-yl]-2-(trifluoromethyl)benzonitrile

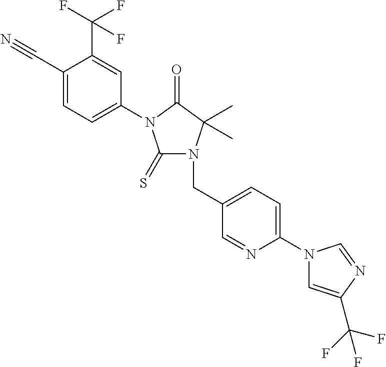

### 23a) Production of Intermediates

### Intermediate 23.1:

### 6-[4-(Trifluoromethyl)-1H-imidazol-1-yl]pyridine-3-carbonitrile

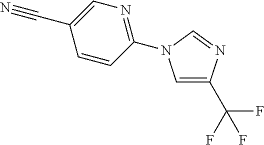

Potassium carbonate (1.02 g; 7.3 mmol) was added under stirring to a solution of 6-chloropyridine-3-carbonitrile (1.02 g; 7.3 mmol) and 4-(trifluoromethyl)-1H-imidazole (1.00 g; 7.3 mmol) in dimethylsulfoxide (7.1 ml) at room temperature. The reaction mixture was stirred at 100° C. for 2 hours. After cooling, the reaction mixture was added to ice water. The precipitate was washed with cool water and dried to yield the desired product (1.49 g; 6.3 mmol).

¹H-NMR (DMSO-d₆): 9.02 (m, 1H), 8.82 (m, 1H), 8.68 (m, 1H), 8.61 (m, 1H), 8.16 (m, 1H).

### b) Production of End Product

Starting from 6-[4-(trifluoromethyl)-1H-imidazol-1-yl]pyridine-3-carbonitrile, Example 23 was prepared analogously to the preparation of Example 1.

¹H-NMR (CDCl₃): 8.58 (m, 1H), 8.36 (m, 1H), 8.09 (m, 1H), 7.99 (m, 2H), 7.93 (m, 1H), 7.81 (m, 1H), 7.42 (m, 1H), 5.15 (s, 2H), 1.56 (s, 6H).

### EXAMPLE 24

### 4-(4,4-Dimethyl-5-oxo-3-{[6-(thien-2-yl)pyridin-3-yl]methyl}-2-thioxoimidazolidin-1-yl)-2-(trifluoromethyl)benzonitrile

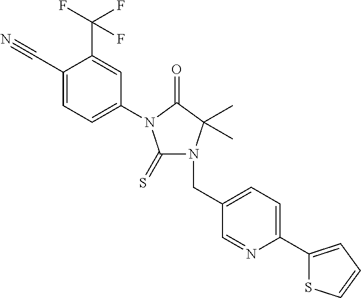

### 24a) Production of Intermediates

Intermediate 24.1: 6-(Thien-2-yl)pyridine-3-carbonitrile

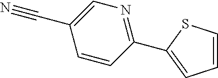

Under argon a solution of sodium carbonate (870 mg, 8.2 mmol) in water (3.6 ml) was added to a solution of 6-chloropyridine-3-carbonitrile (500 mg, 3.6 mmol) and 4,4,5,5-tetramethyl-2-(thien-2-yl)-1,3,2-dioxaborolane (692 mg, 3.3 mmol) in 1,2-dimethoxyethane (7.3 ml). Finally, tetrakis(triphenylphosphine)palladium(0) (417 mg, 0.4 mmol) was added and the batch was stirred in a sealed tube in a microwave oven at 135° C. for 30 min. After cooling, the batch was diluted with ethyl acetate and washed with diluted solutions of 0.5 N sodium hydroxide and sodium chloride. The organic phase was dried over sodium sulfate, filtered and concentrated. The residue was purified by column chromatography (hexane→ethyl acetate) to give the desired product (490 mg; 2.6 mmol).

¹H-NMR (DMSO-d₆): 8.91 (m, 1H), 8.29 (m, 1H), 8.09 (m, 1H), 7.98 (m, 1H), 7.77 (m, 1H), 7.21 (m, 1H).

### b) Production of End Product

Starting from 6-(thien-2-yl)pyridine-3-carbonitrile, Example 24 was prepared analogously to the preparation of Example 1.

¹H-NMR (CDCl₃): 8.61 (m, 1H), 7.99 (m, 1H), 7.93 (m, 1H), 7.88 (m, 1H), 7.80 (m, 1H), 7.67 (m, 1H), 7.59 (m, 1H), 7.42 (m, 1H), 7.13 (m, 1H), 5.11 (s, 2H), 1.52 (s, 6H).

### EXAMPLE 25

### 4-(4,4-Dimethyl-5-oxo-3-{[6-(2-oxoimidazolidin-1-yl)pyridin-3-yl]methyl}-2-thioxoimidazolidin-1-yl)-2-(trifluoromethyl)benzonitrile

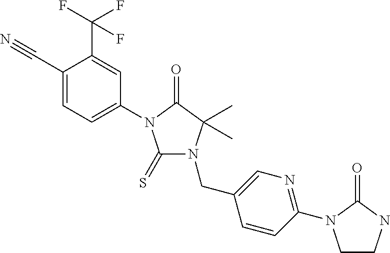

### 25a) Production of Intermediates

### Intermediate 25.1:

### 6-(2-Oxoimidazolidin-1-yl)pyridine-3-carbonitrile

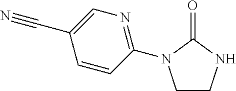

Under stirring a solution of imidazolidin-2-one (2.51 g, 28.0 mmol) in dimethylsulfoxide (6.0 ml) was added dropwise to a suspension of sodium hydride (60%) (0.62 g, 15.4 mmol) in toluene (3 ml) at 0° C. under argon. The batch was warmed to room temperature and a solution of 6-chloropyridine-3-carbonitrile (2.00 g; 14.0 mmol) in dimethylsulfoxide (6 ml) was added. The batch was stirred at 95° C. for 5 hours. After cooling, the reaction mixture was added to ice water and extracted with ethyl acetate (2×). The combined organic phases were filtered using a Whatman filter and concentrated by evaporation. The residue was purified by column chromatography (dichloromethane/ethanol 9:1) to give the desired product (89 mg, 0.5 mmol).

¹H-NMR (CDCl₃): 8.56 (m, 1H), 8.41 (m, 1H), 7.81 (m, 1H), 5.32 (br, 1H), 4.19 (tr, 2H), 3.62 (tr, 2H).

### b) Production of End Product

Starting from 6-(2-oxoimidazolidin-1-yl)pyridine-3-carbonitrile, Example 25 was prepared analogously to the preparation of Example 1.

¹H-NMR (CDCl₃): 8.37 (m, 1H), 8.28 (m, 1H), 7.97 (m, 1H), 7.91 (m, 1H), 7.77 (m, 2H), 5.06 (s, 2H), 4.93 (br, 1H), 4.17 (tr, 2H), 3.59 (tr, 2H), 1.49 (s, 6H).

### EXAMPLE 26

### (R)-4-{4,4-Dimethyl-3-[(6-{[methyl(oxido)phenyl-λ⁶-sulfanylidene]amino}pyridin-3-yl)methyl]-5-oxo-2-thioxoimidazolidin-1-yl}-2-(trifluoromethyl)benzonitrile

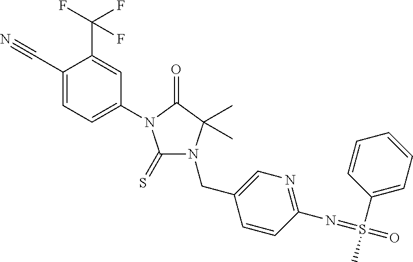

### 26a) Production of Intermediates

### Intermediate 26.1:

### (R)-6-{[Methyl(oxido)phenyl-λ⁶-sulfanylidene]amino}pyridine-3-carbonitrile

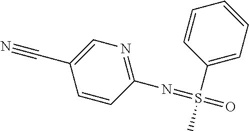

Sodium hydride (60%) (79 mg, 2.0 mmol) was added to a solution of (R)-(−)-S-methyl-S-phenylsulfoximin (560 mg, 3.6 mmol) in toluene (1.0 ml) at 0° C. under argon. A solution of 6-chloropyridine-3-carbonitrile (250 mg; 1.8 mmol) in toluene (2 ml) and DMF (2 ml) was added and the batch was stirred at 95° C. for 6 hours. After cooling, the reaction mixture was added to ice water and extracted with ethyl acetate (2×). The combined organic phases were filtered using a Whatman filter and concentrated by evaporation. The residue was purified by column chromatography (dichloromethane/ethanol 95:5) to give the desired product (87 mg, 0.3 mmol).

¹H-NMR (CDCl₃): 8.31 (m, 1H), 7.99 (m, 2H), 7.67 (m, 2H), 7.57 (m, 2H), 6.89 (m, 1H), 3.40 (s, 3H).

### b) Production of End Product

Starting from (R)-6-{[methyl(oxido)phenyl-λ⁶-sulfanylidene]amino}pyridine-3-carbonitrile, Example 26 was prepared analogously to the preparation of Example 1.

¹H-NMR (CDCl₃): 8.12 (m, 1H), 8.01 (m, 2H), 7.96 (m, 1H), 7.90 (m, 1H), 7.78 (m, 1H), 7.67 (m, 1H), 7.62 (m, 1H), 7.56 (m, 2H), 6.87 (m, 1H), 5.02 (d, 1H), 4.93 (d, 1H), 3.37 (s, 3H), 1.42 (s, 6H).

### EXAMPLE 27

### 4-(4,4-Dimethyl-3-{[6-(5-methyl-1H-pyrazol-1-yl)pyridin-3-yl]methyl}-5-oxo-2-thioxoimidazolidin-1-yl)-2-(trifluoromethyl)benzonitrile

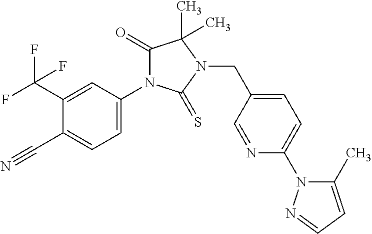

### 27a) Production of Intermediates

### Intermediate 27.1:

### Mixture of 6-(3-methyl-1H-pyrazol-1-yl)pyridine-3-carbonitrile and 6-(5-methyl-1H-pyrazol-1-yl)pyridine-3-carbonitrile

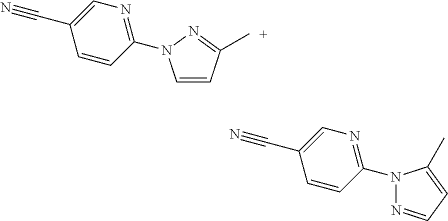

Potassium carbonate (2.00 g; 14.4 mmol) was added under stirring to a solution of 6-chloropyridine-3-carbonitrile (2.00 g; 14.4 mmol) and 3-methyl-1H-pyrazole (1.00 ml; 14.4 mmol) in dimethylsulfoxide (14.0 ml) at room temperature. The reaction mixture was stirred at 100° C. for 2 hours. After cooling the reaction mixture was added to ice water. The precipitate was washed with cool water and dried under vacuum at 50° C. to yield a mixture of the desired products (1.28 g; 7.0 mmol).

### b) Production of End Product

Starting from the mixture of 6-(3-methyl-1H-pyrazol-1-yl)pyridine-3-carbonitrile and 6-(5-methyl-1H-pyrazol-1-yl)pyridine-3-carbonitrile, Example 27 was prepared analogously to the preparation of Example 1. The product was isolated using preparative HPLC.
System: Dionex: Pump P 580, Gilson: Liquid Handler 215, Knauer: UV-Detector K-2501
Column: Chiralpak IA 5 μm 250×30 mm
Solvent: Ethanol/methanol 50:50±0.1% diethylamine
Flow: 25 mL/min
Temperature: Room temperature
Detection: UV 254 nm
Retention 11.0-12.1 minutes

¹H-NMR (CDCl₃): 8.51 (m, 1H), 7.98 (m, 1H), 7.92 (m, 3H), 7.82 (m, 1H), 7.59 (m, 1H), 6.20 (m, 1H), 5.15 (s, 2H), 2.70 (s, 3H), 1.52 (s, 6H).

### EXAMPLE 28

### 4-(3-{[6-(2,2-Difluoro-3-hydroxypropoxy)pyridin-3-yl]methyl}-4,4-dimethyl-5-oxo-2-thioxoimidazolidin-1-yl)-2-(trifluoromethyl)benzonitrile

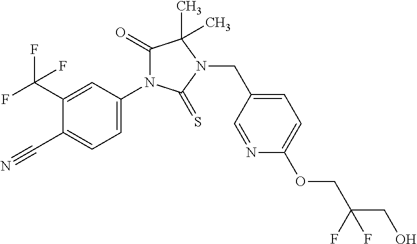

### 28a) Production of Intermediates

### Intermediate 28.1:

### 6-(2,2-Difluoro-3-hydroxypropoxy)pyridine-3-carbonitrile

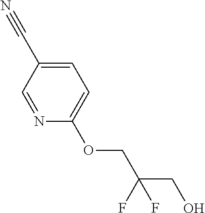

Sodium hydride (60%; 428 mg) was added to a solution of 2,2-difluoropropane-1,3-diol (1000 mg; 8.9 mmol, purchased from SALTIGO Fluorine Team, Leverkusen, Germany) in N,N-dimethylformamide (20.0 ml) and the batch was stirred for 1 hour at room temperature. A solution of 6-chloropyridine-3-carbonitrile (618 mg, 4.5 mmol) in N,N-dimethylformamide (5.0 ml) was added and the batch was stirred over night at room temperature. The mixture was diluted with ice and a diluted solution of sodium chloride and extracted with ethyl acetate (3×). The combined organic phases were washed with a diluted sodium chloride solution, dried over sodium sulfate and filtered. The filtrate was concentrated in vacuo and the residue was purified by column chromatography (hexane→hexane/ethyl acetate 6:4) to give the desired product (596 mg; 2.8 mmol).

¹H-NMR (CDCl₃): 8.49 (m, 1H), 7.88 (m, 1H), 6.95 (m, 1H), 4.72 (tr, 2H), 3.88 (tr d, 2H), 2.57 (tr, 1H).

### b) Production of End Product

Starting from 6-(2,2-difluoro-3-hydroxypropoxy)pyridine-3-carbonitrile, Example 28 was prepared analogously to the preparation of Example 1.

¹H-NMR (CDCl₃): 8.19 (m, 1H), 7.98 (m, 1H), 7.92 (m, 1H), 7.88 (m, 1H), 7.80 (m, 1H), 6.89 (m, 1H), 5.06 (s, 2H), 4.66 (tr, 2H), 3.80 (m, 3H), 1.51 (s, 6H).

### EXAMPLE 29

### 4-(4,4-Dimethyl-5-oxo-2-thioxo-3-{[6-(1H-1,2,3-triazol-1-yl)pyridin-3-yl]methyl}imidazolidin-1-yl)-1)-2-(trifluoromethyl)benzonitrile

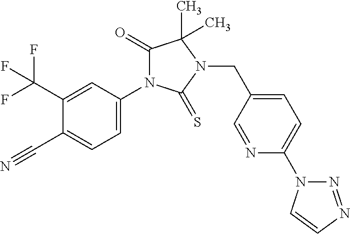

### 29a) Production of Intermediates

### Intermediates 29.1:

### 6-(1H-1,2,3-Triazol-1-yl)pyridine-3-carbonitrile and 6-(1H-1,2,3-triazol-2-yl)pyridine-3-carbonitrile

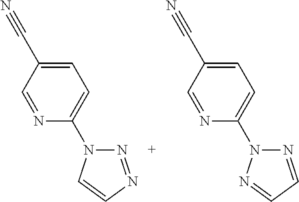

Potassium carbonate (2.00 g; 14.4 mmol) was added under stirring to a solution of 6-chloropyridine-3-carbonitrile (2.00 g; 14.4 mmol) and 1H-1,2,3-triazole (0.84 ml; 14.4 mmol) in dimethylsulfoxide (14.0 ml) at room temperature. The reaction mixture was stirred at 100° C. for 4 hours. After cooling the reaction mixture was added to ice water. The precipitate was washed with cool water and dried under vacuum. The residue was purified by column chromatography (hexane/ethyl acetate 1:1) to give the products 6-(1H-1,2,3-triazol-1-yl)pyridine-3-carbonitrile (678 mg, 4.0 mmol) and 6-(1H-1,2,3-triazol-2-yl)pyridine-3-carbonitrile (360 mg, 2.1 mmol).

### 6-(1H-1,2,3-Triazol-1-yl)pyridine-3-carbonitrile

¹H-NMR (CDCl₃): 8.81 (m, 1H), 8.63 (m, 1H), 8.40 (m, 1H), 8.21 (m, 1H), 7.88 (m, 1H).

### 6-(1H-1,2,3-triazol-2-yl)pyridine-3-carbonitrile

¹H-NMR (CDCl₃): 8.88 (m, 1H), 8.24 (m, 1H), 8.15 (m, 1H), 7.98 (s, 2H).

### b) Production of End Product

Starting from 6-(1H-1,2,3-triazol-1-yl)pyridine-3-carbonitrile, Example 29 was prepared analogously to the preparation of Example 1.

¹H-NMR (CDCl₃): 8.59 (m, 2H), 8.24 (m, 1H), 8.08 (m, 1H), 7.99 (m, 1H), 7.93 (m, 1H), 7.83 (m, 2H), 5.17 (m, 2H), 1.55 (s, 6H).

### EXAMPLE 30

### 4-(4,4-Dimethyl-5-oxo-2-thioxo-3-{[6-(2H-1,2,3-triazol-2-yl)pyridin-3-yl]methyl}imidazolidin-1-yl)-2-(trifluoromethyl)benzonitrile

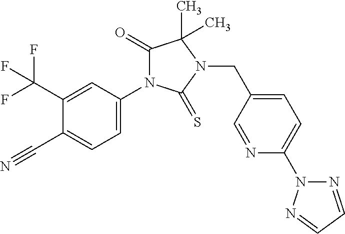

### b) Production of End Product

Starting from 6-(1H-1,2,3-triazol-2-yl)pyridine-3-carbonitrile, Example 30 was prepared analogously to the preparation of Example 1.

¹H-NMR (CDCl₃): 8.63 (s, 1H), 8.12 (m, 2H), 7.99 (m, 1H), 7.92 (m, 3H), 7.81 (m, 1H), 5.18 (s, 2H), 1.54 (s, 6H).

### EXAMPLE 31

### 4-(4,4-Dimethyl-5-oxo-3-{[6-(1H-tetrazol-1-yl)pyridin-3-yl]methyl}-2-thioxoimidazolidin-1-yl)-2-(trifluoromethyl)benzonitrile

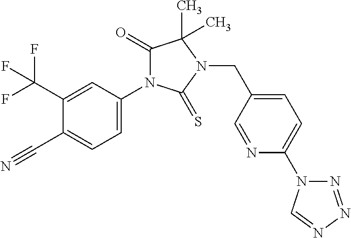

### 31a) Production of Intermediates

### Intermediate 31.1:

### 6-(1H-Tetrazol-1-yl)pyridine-3-carbonitrile

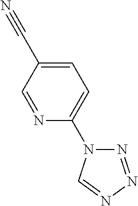

Potassium carbonate (2.00 g; 14.4 mmol) was added under stirring to a solution of 6-chloropyridine-3-carbonitrile (2.00 g; 14.4 mmol) and 1H-tetrazole (1.01 g; 14.4 mmol) in dimethylsulfoxide (14.0 ml) at room temperature. The reaction mixture was stirred at 100° C. for 4 hours. After cooling the reaction mixture was added to ice water. The precipitate was washed with cool water and dried under vacuum. The crude product was used without further purifications.

### b) Production of End Product

Starting from 6-(1H-tetrazol-1-yl)pyridine-3-carbonitrile, Example 31 was prepared analogously to the preparation of Example 1.

¹H-NMR (CDCl₃): 9.53 (s, 1H), 8.62 (m, 1H), 8.14 (m, 2H), 7.99 (m, 1H), 7.92 (m, 1H), 7.81 (m, 1H), 5.18 (s, 2H), 1.57 (s, 6H).

### EXAMPLE 32

### 4-(3-{[6-(4,5-Dichloro-1H-imidazol-1-yl)pyridin-3-yl]methyl}-4,4-dimethyl-5-oxo-2-thioxoimidazolidin-1-yl)-2-(trifluoromethyl)benzonitrile

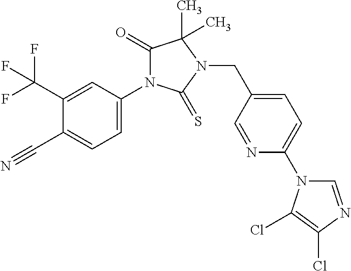

### 32a) Production of Intermediates

### Intermediate 32.1:

### 6-(4,5-Dichloro-1H-imidazol-1-yl)pyridine-3-carbonitrile

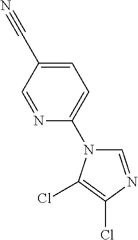

Potassium carbonate (2.00 g; 14.4 mmol) was added under stirring to a solution of 6-chloropyridine-3-carbonitrile (2.00 g; 14.4 mmol) and 4,5-dichloro-1H-imidazole (1.98 g; 14.4 mmol) in dimethylsulfoxide (14.0 ml) at room temperature. The reaction mixture was stirred at 100° C. for 2 hours. After cooling the reaction mixture was added to ice water. The precipitate was washed with cool water and dried under vacuum to give the desired product.

¹H-NMR (CDCl₃): 8.85 (m, 1H), 8.20 (m, 1H), 8.18 (s, 1H), 7.84 (m, 1H).

### b) Production of End Product

Starting from 6-(4,5-dichloro-1H-imidazol-1-yl)pyridine-3-carbonitrile, Example 32 was prepared analogously to the preparation of Example 1.

¹H-NMR (CDCl₃): 8.62 (m, 1H), 8.09 (m, 1H), 8.06 (s, 1H), 7.99 (m, 1H), 7.92 (m, 1H), 7.81 (m, 1H), 7.63 (m, 1H), 5.16 (s, 2H), 1.57 (s, 6H).

### EXAMPLE 33

### 4-[4,4-Dimethyl-5-oxo-2-thioxo-3-({6-[3-(trifluoromethyl)-1H-1,2,4-triazol-1-yl]pyridin-3-yl}methyl)imidazolidin-1-yl]-2-(trifluoromethyl)benzonitrile

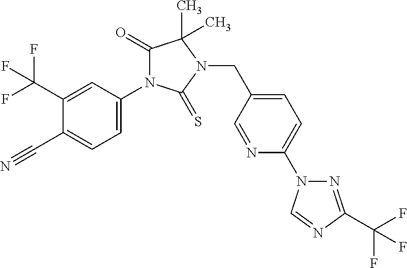

### 33a) Production of Intermediates

### Intermediate 33.1:

### 6-[3-(Trifluoromethyl)-1H-1,2,4-triazol-1-yl]pyridine-3-carbonitrile

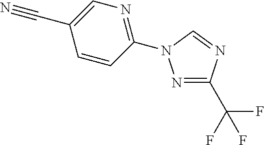

Potassium carbonate (2.01 g; 14.6 mmol) was added under stirring to a solution of 6-chloropyridine-3-carbonitrile (2.02 g; 14.6 mmol) and 3-(trifluoromethyl)-1H-1,2,4-triazole (2.00 g; 14.6 mmol) in dimethylsulfoxide (14.1 ml) at room temperature. The reaction mixture was stirred at 100° C. for 2 hours. After cooling the reaction mixture was added to ice water. The precipitate was washed with cool water and dried under vacuum to give the desired product (3.14 g, 13.1 mmol).

¹H-NMR (DMSO-d₆): 9.75 (m, 1H), 9.08 (m, 1H), 8.59 (m, 1H), 8.07 (m, 1H).

### b) Production of End Product

Starting from 6-[3-(trifluoromethyl)-1H-1,2,4-triazol-1-yl]pyridine-3-carbonitrile, Example 33 was prepared analogously to the preparation of Example 1.

¹H-NMR (CDCl₃): 9.23 (m, 1H), 8.57 (m, 1H), 8.09 (m, 1H), 7.98 (m, 2H), 7.92 (m, 1H), 7.81 (m, 1H), 5.17 (s, 2H), 1.55 (s, 6H).

### EXAMPLE 34

### 4-[3-({6-[4-(Hydroxymethyl)-1H-imidazol-1-yl]pyridin-3-yl}methyl)-4,4-dimethyl-5-oxo-2-thioxoimidazolidin-1-yl]-2-(trifluoromethyl)benzonitrile

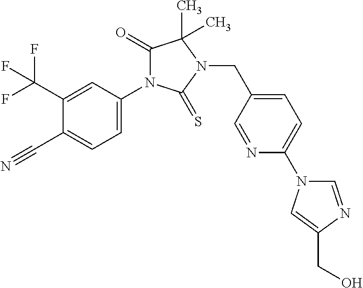

### 34a) Production of Intermediates

### Intermediate 34.1:

### 6-[4-(Hydroxymethyl)-1H-imidazol-1-yl]pyridine-3-carbonitrile

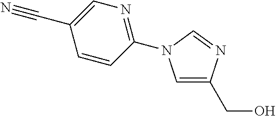

Potassium carbonate (2.00 g; 14.4 mmol) was added under stirring to a solution of 6-chloropyridine-3-carbonitrile (2.02 g; 14.6 mmol) and 1H-imidazole-4-methanol (1.42 g; 14.4 mmol) in dimethylsulfoxide (14.0 ml) at room temperature. The reaction mixture was stirred at 100° C. for 2 hours. After cooling the reaction mixture was added to ice water. and extracted with ethyl acetate (3×). The combined organic phases were washed with a diluted sodium chloride solution, dried over sodium sulfate and filtered. The filtrate was concentrated in vacuo and the residue was purified by column chromatography (ethyl acetate) to give the desired product (724 mg; 3.6 mmol).

¹H-NMR (DMSO-d₆): 8.93 (m, 1H), 8.55 (m, 1H), 8.46 (m, 1H), 8.00 (m, 1H), 7.82 (m, 1H), 5.07 (tr, 1H), 4.38 (d, 2H).

### b) Production of End Product

Starting from 6-[4-(hydroxymethyl)-1H-imidazol-1-yl]pyridine-3-carbonitrile, Example 34 was prepared analogously to the preparation of Example 1.

¹H-NMR (DMSO-d₆): 8.61 (m, 1H), 8.46 (m, 1H), 8.38 (m, 1H), 8.33 (m, 1H), 8.09 (m, 2H), 7.77 (m, 2H), 5.16 (s, 2H), 5.00 (tr, 1H), 4.43 (d, 2H), 1.52 (s, 6H).

### EXAMPLE 35

### 4-[4,4-Dimethyl-5-oxo-3-({6-[2-(2-oxoimidazolidin-1-yl)ethoxyl]pyridin-3-yl}methyl)-2-thioxoimidazolidin-1-yl]-2-(trifluoromethyl)benzonitrile

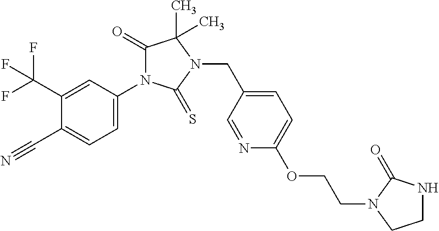

### 35a) Production of Intermediates

### Intermediate 35.1:

### 6-[2-(2-Oxoimidazolidin-1-yl)ethoxy]pyridine-3-carbonitrile

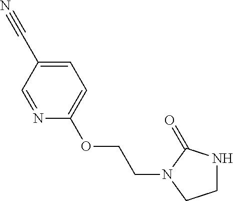

1-(2-Hydroxyethyl)imidazolidin-2-one (1.08 g, 8.3 mmol) was added to a suspension of sodium hydride (60%) (0.37 g, 9.1 mmol) in toluene (3.3 ml) under argon and stirred for 10 minutes at room temperature. 6-Chloropyridine-3-carbonitrile (1.15 g; 8.3 mmol) was added and the batch was stirred at 95° C. for 4 hours. After cooling, the reaction mixture was added to ice water and extracted with ethyl acetate (2×). The combined organic phases were dried (Na₂SO₄), filtered and concentrated by evaporation. The residue was purified by column chromatography (dichloromethane/ethanol 9:1) to give the desired product (0.70 mg, 3.0 mmol).

¹H-NMR (DMSO-d₆): 8.68 (m, 1H), 8.14 (m, 1H), 7.00 (m, 1H), 6.33 (br, 1H), 4.44 (tr, 2H), 3.42 (m, 4H), 3.21 (tr, 2H).

### b) Production of End Product

Starting from 6-[2-(2-oxoimidazolidin-1-yl)ethoxy]pyridine-3-carbonitrile, Example 35 was prepared analogously to the preparation of Example 1.

¹H-NMR (CDCl₃): 8.19 (m, 1H), 7.97 (m, 1H), 7.91 (m, 1H), 7.79 (m, 2H), 6.76 (m, 1H), 5.05 (s, 2H), 4.45 (tr, 2H), 3.60 (m, 4H), 3.41 (tr, 2H), 1.50 (s, 6H).

### EXAMPLE 36

### {4-[5-({3-[4-Cyano-3-(trifluoromethyl)phenyl]-5,5-dimethyl-4-oxo-2-thioxoimidazolidin-1-yl}methyl)pyridin-2-yl]-1-oxido-1λ⁶,4-thiomorpholin-1-ylidene}cyanamide

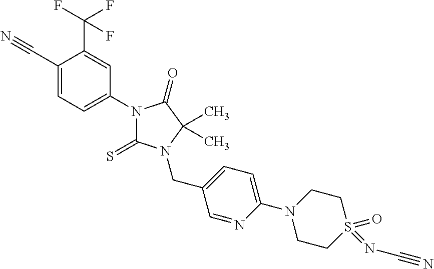

4-Dimethylaminopyridine (6.0 mg, 0.049 mmol) and bromocyane (9.4 mg, 0.089 mmol) were added to a solution of 4-(3-{[6-(1-imino-1-oxido-1λ⁶-thiomorpholin-4-yl)pyridin-3-yl]methyl}-4,4-dimethyl-5-oxo-2-thioxoimidazolidin-1-yl)-2-(trifluoromethyl)benzonitrile (24.0 mg, 0.045 mmol) in dichloromethane (0.22 ml). The batch was stirred for 4 hours at room temperature and finally concentrated. The residue was purified by preparative HPLC to give the desired product (12.0 mg, 0.020 mmol).
System: Agilent: Prep 1200, 2×Prep Pump, DLA, MWD, ELSD, Prep FC
Column: XBrigde C18 5 μm 150×19 mm
Solvent A: H₂O/0.1% HCOOH
Solvent B: Methanol
Gradient: 0-12.5 min 50-80% B, 12.5-15 min 80-100% B
Flow: 21 mL/min
Temperature: Room temperature
Detection: MWD 214 nm/ELSD
Retention 7.0-8.5 minutes

¹H-NMR (CDCl₃): 8.29 (m, 1H), 7.97 (m, 1H), 7.90 (m, 1H), 7.86 (m, 1H), 7.79 (m, 1H), 6.80 (m, 1H), 5.02 (s, 2H), 4.54 (m, 2H), 3.89 (m, 2H), 3.54 (m, 2H), 3.33 (m, 2H), 1.52 (s, 6H).

### EXAMPLE 37

### 4-(3-{[6-(2-methylpropoxy)-2-methylpyridin-3-yl]methyl}-4,4-dimethyl-5-oxo-2-thioxoimidazolidin-1-yl)-2-(trifluoromethyl)benzonitrile

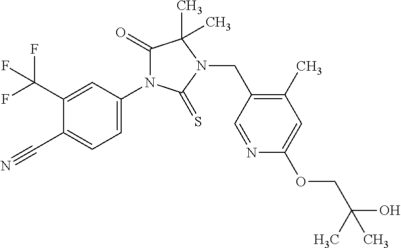

### 37a) Production of Intermediates

### Intermediate 37.1:

### 6-(2-Hydroxy-2-methylpropoxy)-2-methylpyridine-3-carbonitrile

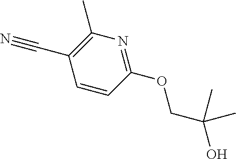

Sodium hydride (60%) (314 mg, 7.9 mmol) was added to a solution of 2-methylpropane-1,2-diol (591 mg; 6.6 mmol) in N,N-dimethylformamide (60.5 ml) and the batch was stirred for 1 hour at room temperature. A solution of 6-chloro-2-methylpyridine-3-carbonitrile (1000 mg, 6.6 mmol) in N,N-dimethylformamide (6.0 ml) was added and the batch was stirred over night at room temperature. The mixture was diluted with ice and a diluted solution of sodium chloride and extracted with ethyl acetate (3×). The combined organic phases were washed with a diluted sodium chloride solution, dried over sodium sulfate and filtered. The filtrate was concentrated in vacuo and the residue was purified by column chromatography (hexane→hexane/ethyl acetate 1:1) to give the desired product (489 mg; 2.4 mmol).

¹H-NMR (CDCl₃): 7.72 (m, 1H), 6.68 (m, 1H), 4.24 (s, 2H), 2.72 (s, 1H), 2.64 (s, 3H), 1.31 (s, 6H).

### 37b) Production of End Product

Starting from 6-(2-hydroxy-2-methylpropoxy)-2-methylpyridine-3-carbonitrile, Example 37 was prepared analogously to the preparation of Example 1.

¹H-NMR (CDCl₃): 7.98 (m, 1H), 7.93 (m, 1H), 7.82 (m, 1H), 7.58 (m, 1H), 6.68 (m, 1H), 5.06 (s, 2H), 4.22 (s, 2H), 2.55 (s, 3H), 1.47 (s, 6H), 1.32 (s, 6H).

### EXAMPLE 38

### 4-(4,4-Dimethyl-3-{[6-(5-methyl-1H-tetrazol-1-yl)pyridin-3-yl]methyl}-5-oxo-2-thioxoimidazolidin-1-yl)-2-(trifluoromethyl)benzonitrile

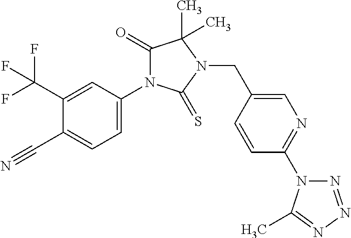

### 38a) Production of Intermediates

### Intermediate 38.1:

### Mixture of 6-(5-methyl-1H-tetrazol-2-yl)pyridine-3-carbonitrile and 6-(5-methyl-1H-tetrazol-1-yl)pyridine-3-carbonitrile

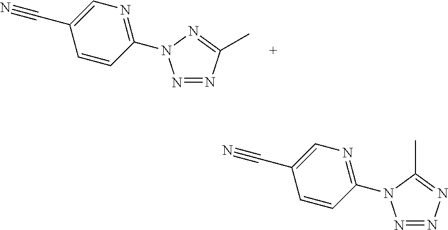

Potassium carbonate (1.70 g; 12.3 mmol) was added under stirring to a solution of 6-chloropyridine-3-carbonitrile (1.71 g; 12.3 mmol) and 5-methyl-1H-tetrazole (0.87 ml; 12.3 mmol) in dimethylsulfoxide (11.9 ml) at room temperature. The reaction mixture was stirred at 100° C. for 2 hours. After cooling the reaction mixture was added to ice water and extracted with ethyl acetate (2×). The combined organic phases were dried (Na₂SO₄), filtered and concentrated. The residue was purified by column chromatography (ethyl acetate) to yield a mixture of the desired products (1.27 g; 6.8 mmol) that was used without further purification.

### b) Production of End Product

Starting from the mixture of 6-(5-methyl-1H-tetrazol-2-yl)pyridine-3-carbonitrile and 6-(5-methyl-1H-tetrazol-1-yl)pyridine-3-carbonitrile, Example 38 was prepared analogously to the preparation of Example 1. The product was isolated using preparative HPLC.
System: Dionex: Pump P 580, Gilson: Liquid Handler 215, Knauer: UV-Detector K-2501
Column: Chiralpak 1B 5 μm 250×30 mm
Solvent: Hexane/ethanol 50:50±0.1% diethylamine
Flow: 30 mL/min
Temperature: Room temperature
Detection: UV 254 nm
Retention 7.8-10.2 minutes

¹H-NMR (CDCl₃): 8.65 (m, 1H), 8.10 (m, 2H), 7.99 (m, 1H), 7.93 (m, 1H), 7.82 (m, 1H), 5.18 (s, 2H), 2.96 (s, 3H), 1.57 (s, 6H).

### EXAMPLE 39

### In Vitro Pharmacological Properties of the Compounds

TABLE 1

| | | AR | AR | AR |
|---|---|---|---|---|
| | | (wildtype) | (wildtype) | W741L |
| | | antagonism | agonism | antagonism |
| Example | Name of compound | IC₅₀ (mol/l)¹ | EC₅₀ (mol/l)² | IC₅₀ (mol/l)³ |
| Comparative Data; example 12 of U.S. patent U.S. RE 35,956 | 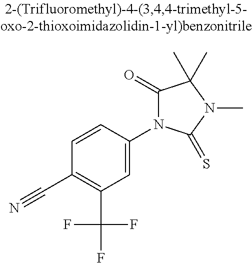 | 1.0E−5 | 7.97E−9 | 1.59E−7 |
| Comparative Data; example 77 of U.S. patent U.S. RE 35,956 | 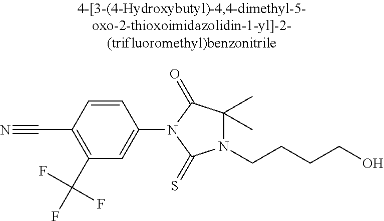 | 2.15E−9 | 8.13E−9 | 8.97E−8 |
| Comparative data⁴ | 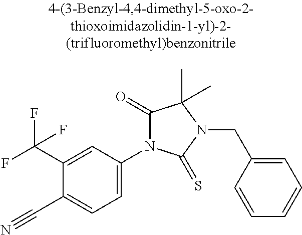 | 1.5E−8 | 4.64E−8 | 1.66E−7 |
| Comparative data⁵ | 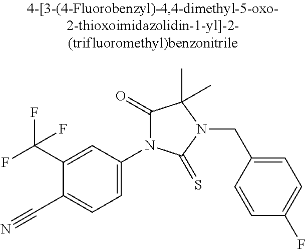 | >1.0E−5 | 1.79E−9 | >1.0E−6 |
| Comparative data^{⁶} | 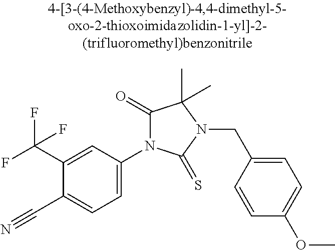 | 1.34E−7 | 6.81E−6 | 3.79E−7 |
| Comparative data⁷ | 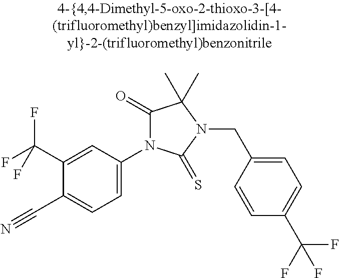 | 3.0E−7 | 6.8E−6 | 5.78E−7 |
| 1 | 4-(3-{[6-(1H-Imidazol-1-yl)pyridin-3- | 5.97E−8 | >1.0E−5 | 1.73E−7 |
| | yl]methyl}-4,4-dimethyl-5-oxo-2- | | | |
| | thioxoimidazolidin-1-yl)-2- | | | |
| | (trifluoromethyl)benzonitrile | | | |
| 2 | 4-(4,4-Dimethyl-5-oxo-2-thioxo-3-{[6- | 6.84E−8 | 5.43E−6 | 8.67E−8 |
| | (trifluoromethyl)pyridin-3- | | | |
| | yl]methyl}imidazolidin-1-yl)-2- | | | |
| | (trifluoromethyl)benzonitrile | | | |
| 3 | 4-[4,4-Dimethyl-3-({6-[2-(morpholin-4- | 4.58E−8 | >1.0E−5 | 1.18E−7 |
| | yl)ethoxy]pyridin-3-yl}methyl)-5-oxo-2- | | | |
| | thioxoimidazolidin-1-yl]-2- | | | |
| | (trifluoromethyl)benzonitrile | | | |
| 4 | 4-(4,4-Dimethyl-3-{[6-(morpholin-4- | 7.58E−8 | >1.0E−5 | 8.92E−8 |
| | yl)pyridin-3-yl]methyl}-5-oxo-2- | | | |
| | thioxoimidazolidin-1-yl)-2- | | | |
| | (trifluoromethyl)benzonitrile | | | |
| 5 | 4-(4,4-Dimethyl-5-oxo-3-{[6-(tetrahydro- | 5.02E−8 | >1.0E−5 | 4.97E−8 |
| | 2H-pyran-4-yloxy)pyridin-3-yl]methyl}-2- | | | |
| | thioxoimidazolidin-1-yl)-2- | | | |
| | (trifluoromethyl)benzonitrile | | | |
| 6 | 4-(3-{[4-Amino-2-(morpholin-4- | 3.45E−8 | 5.0E−6 | 9.28E−8 |
| | yl)pyrimidin-5-yl]methyl}-4,4-dimethyl-5- | | | |
| | oxo-2-thioxoimidazolidin-1-yl)-2- | | | |
| | (trifluoromethyl)benzonitrile | | | |
| 7 | 4-(4,4-Dimethyl-3-{[6-(2- | 7.56E−8 | >1.0E−5 | 4.91E−8 |
| | methylmorpholin-4-yl)pyridin-3- | | | |
| | yl]methyl}-5-oxo-2-thioxoimidazolidin-1- | | | |
| | yl)-2-(trifluoromethyl)benzonitrile | | | |
| 8 | 4-{3-[(6-Methoxypyridin-3-yl)methyl]-4,4- | 4.93E−8 | 8.86E−6 | 4.92E−8 |
| | dimethyl-5-oxo-2-thioxoimidazolidin-1- | | | |
| | yl}-2-(trifluoromethyl)benzonitrile | | | |
| 9 | 4-(3-{[6-(1-Imino-1-oxido-1λ⁶- | 5.9E−8 | >1.0E−5 | 2.75E−7 |
| | thiomorpholin-4-yl)pyridin-3-yl]methyl}- | | | |
| | 4,4-dimethyl-5-oxo-2-thioxoimidazolidin- | | | |
| | 1-yl)-2-(trifluoromethyl)benzonitrile | | | |
| 10 | 4-(3-{[6-(2-Hydroxy-2- | 6.89E−8 | >1.0E−5 | 2.85E−7 |
| | methylpropoxy)pyridin-3-yl]methyl}-4,4- | | | |
| | dimethyl-5-oxo-2-thioxoimidazolidin-1- | | | |
| | yl)-2-(trifluoromethyl)benzonitrile | | | |
| 11 | 4-(3-{[6-(2-Methoxyethoxy)pyridin-3- | 1.0E−7 | >1.0E−5 | Not determ. |
| | yl]methyl}-4,4-dimethyl-5-oxo-2- | | | |
| | thioxoimidazolidin-1-yl)-2- | | | |
| | (trifluoromethyl)benzonitrile | | | |
| 12 | 4-(4,4-Dimethyl-3-{[6-(4-methyl-1,4- | 1.84E−7 | >1.0E−5 | Not determ. |
| | diazepan-1-yl)pyridin-3-yl]methyl}-5-oxo- | | | |
| | 2-thioxoimidazolidin-1-yl)-2- | | | |
| | (trifluoromethyl)benzonitrile | | | |
| 13 | 4-(4,4-Dimethyl-3-{[2-methyl-6- | 1.86E−7 | >1.0E−5 | 9.37E−8 |
| | (trifluoromethyl)pyridin-3-yl]methyl}-5- | | | |
| | oxo-2-thioxoimidazolidin-1-yl)-2- | | | |
| | (trifluoromethyl)benzonitrile | | | |
| 14 | 4-[3-({6-[4-(Hydroxymethyl)piperidin-1- | 3.72E−8 | 6.0E−6 | Not determ. |
| | yl]pyridin-3-yl}methyl)-4,4-dimethyl-5- | | | |
| | oxo-2-thioxoimidazolidin-1-yl]-2- | | | |
| | (trifluoromethyl)benzonitrile | | | |
| 15 | 4-(4,4-Dimethyl-3-{[6-(2-methyl-1H- | 9.78E−8 | >1.0E−5 | 1.32E−8 |
| | imidazol-1-yl)pyridin-3-yl]methyl}-5-oxo- | | | |
| | 2-thioxoimidazolidin-1-yl)-2- | | | |
| | (trifluoromethyl)benzonitrile | | | |
| 16 | 4-(3-{[6-(4,4-Dimethyl-2-oxopyrrolidin-1- | 5.96E−8 | >1.0E−5 | 7.85E−8 |
| | yl)pyridin-3-yl]methyl}-4,4-dimethyl-5- | | | |
| | oxo-2-thioxoimidazolidin-1-yl)-2- | | | |
| | (trifluoromethyl)benzonitrile | | | |
| 17 | 4-(3-{[2-(1H-Imidazol-1-yl)pyrimidin-5- | 1.29E−7 | >1.0E−5 | 2.06E−8 |
| | yl]methyl}-4,4-dimethyl-5-oxo-2- | | | |
| | thioxoimidazolidin-1-yl)-2- | | | |
| | (trifluoromethyl)benzonitrile | | | |
| 18 | 4-(4,4-Dimethyl-3-{[6-(1-methyl-1H- | 4.77E−8 | 9.57E−6 | 9.46E−8 |
| | pyrazol-4-yl)pyridin-3-yl]methyl}-5-oxo- | | | |
| | 2-thioxoimidazolidin-1-yl)-2- | | | |
| | (trifluoromethyl)benzonitrile | | | |
| 19 | 4-(4,4-Dimethyl-3-{[6-(4-methyl-1H- | 1.61E−7 | >1.0E−5 | 2.01E−7 |
| | imidazol-1-yl)pyridin-3-yl]methyl}-5-oxo | | | |
| | 2-thioxoimdazolidin-1-yl)-2- | | | |
| | (trifluoromethyl)benzonitrile | | | |
| 20 | 4-(4,4-Dimethyl-3-{[6-(1-methyl-1H- | 1.18E−7 | >1.0E−5 | 2.33E−7 |
| | pyrazol-5-yl)pyridin-3-yl]methyl}-5-oxo- | | | |
| | 2-thioxoimidazolidin-1-yl)-2- | | | |
| | (trifluoromethyl)benzonitrile | | | |
| 21 | 4-(3-{[6-(4-Chloro-2-methyl-1H-imidazol- | 1.35E−7 | >1.0E−5 | 2.96E−7 |
| | 1-yl)pyridin-3-yl]methyl}-4,4-dimethyl-5- | | | |
| | oxo-2-thioxoimidazolidin-1-yl)-2- | | | |
| | (trifluoromethyl)benzonitrile | | | |
| 22 | 4-[4,4-Dimethyl-3-({6-[1-(methylimino)-1- | 8.3E−8 | >1.0E−5 | 5.74E−8 |
| | oxido-1λ⁶-thiomorpholin-4-yl]pyridin-3- | | | |
| | yl}methyl)-5-oxo-2-thioxoimidazolidin-1- | | | |
| | yl]-2-(trifluoromethyl)benzonitrile | | | |
| 23 | 4-[4,4-Dimethyl-5-oxo-2-thioxo-3-({6-[4- | 2.04E−7 | >1.0E−5 | 8.3E−8 |
| | (trifluoromethyl)-1H-imidazol-1- | | | |
| | yl]pyridin-3-yl}methyl)imidazolidin-1-yl]- | | | |
| | 2-(trifluoromethyl)benzonitrile | | | |
| 24 | 4-(4,4-Dimethyl-5-oxo-3-{[6-(thien-2- | 2.31E−7 | >1.0E−5 | 2.02E−7 |
| | yl)pyridin-3-yl]methyl}-2- | | | |
| | thioxoimidazolidin-1-yl)-2- | | | |
| | (trifluoromethyl)benzonitrile | | | |
| 25 | 4-(4,4-Dimethyl-5-oxo-3-{[6-(2- | 8.32E−8 | >1.0E−5 | 2.46E−7 |
| | oxoimidazolidin-1-yl)pyridin-3- | | | |
| | yl]methyl}-2-thioxoimidazolidin-1-yl)-2- | | | |
| | (trifluoromethyl)benzonitrile | | | |
| 26 | (R)-4-{4,4-Dimethyl-3-[(6- | 2.91E−7 | >1.0E−5 | 2.11E−7 |
| | {[methyl(oxido)phenyl-λ⁶- | | | |
| | sulfanylidene]amino}pyridin-3-yl)methyl]- | | | |
| | 5-oxo-2-thioxoimidazolidin-1-yl}-2- | | | |
| | (trifluoromethyl)benzonitrile | | | |
| 27 | 4-(4,4-Dimethyl-3-{[6-(5-methyl-1H- | 2.92E−7 | >1.0E−5 | Not |
| | pyrazol-1-yl)pyridin-3-yl]methyl}-5-oxo- | | | determined |
| | 2-thioxoimidazolidin-1-yl)-2- | | | |
| | (trifluoromethyl)benzonitrile | | | |
| 28 | 4-(3-{[6-(2,2-Difluoro-3- | 4.32E−8 | >1.0E−5 | Not |
| | hydroxypropoxy)pyridin-3-yl]methyl}-4,4- | | | determined |
| | dimethyl-5-oxo-2-thioxoimidazolidin-1- | | | |
| | yl)-2-(trifluoromethyl)benzonitrile | | | |
| 29 | 4-(4,4-Dimethyl-5-oxo-2-thioxo-3-{[6- | 3.73E−8 | >1.0E−5 | Not |
| | (1H-1,2,3-triazol-1-yl)pyridin-3- | | | determined |
| | yl]methyl}imidazolidin-1-yl)-2- | | | |
| | (trifluoromethyl)benzonitrile | | | |
| 30 | 4-(4,4-Dimethyl-5-oxo-2-thioxo-3-{[6- | 1.39E−7 | >1.0E−5 | Not |
| | (2H-1,2,3-triazol-2-yl)pyridin-3- | | | determined |
| | yl]methyl}imidazolidin-1-yl)-2- | | | |
| | (trifluoromethyl)benzonitrile | | | |
| 31 | 4-(4,4-Dimethyl-5-oxo-3-{[6-(1H-tetrazol- | 1.42E−7 | >1.0E−5 | 1.0E−6 |
| | 1-yl)pyridin-3-yl]methyl}-2- | | | |
| | thioxoimidazolidin-1-yl)-2- | | | |
| | (trifluoromethyl)benzonitrile | | | |
| 32 | 4-(3-{[6-(4,5-Dichloro-1H-imidazol-1- | 9.98E−8 | >1.0E−5 | Not |
| | yl)pyridin-3-yl]methyl}-4,4-dimethyl-5- | | | determined |
| | oxo-2-thioxoimidazolidin-1-yl)-2- | | | |
| | (trifluoromethyl)benzonitrile | | | |
| 33 | 4-[4,4-Dimethyl-5-oxo-2-thioxo-3-({6-[3- | 1.57E−7 | >1.0E−5 | Not |
| | (trifluoromethyl)-1H-1,2,4-triazol-1- | | | determined |
| | yl]pyridin-3-yl}methyl)imidazolidin-1-yl]- | | | |
| | 2-(trifluoromethyl)benzonitrile | | | |
| 34 | 4-[3-({6-[4-(Hydroxymethyl)-1H- | 1.3E−7 | >1.0E−5 | 2.59E−7 |
| | imidazol-1-yl]pyridin-3-yl}methyl)-4,4- | | | |
| | dimethyl-5-oxo-2-thioxoimidazolidin-1- | | | |
| | yl]-2-(trifluoromethyl)benzonitrile | | | |
| 35 | 4-[4,4-Dimethyl-5-oxo-3-({6-[2-(2- | 7.58E−8 | >1.0E-5 | 4.46E−8 |
| | oxoimidazolidin-1-yl)ethoxy]pyridin-3- | | | |
| | yl}methyl)-2-thioxoimidazolidin-1-yl]-2- | | | |
| | (trifluoromethyl)benzonitrile | | | |
| 36 | {4-[5-({3-[4-Cyano-3- | 1.88E−7 | >1.0E−5 | 1.0E−6 |
| | (trifluoromethyl)phenyl]-5,5-dimethyl-4- | | | |
| | oxo-2-thioxoimidazolidin-1- | | | |
| | yl}methyl)pyridin-2-yl]-1-oxido-1λ⁶- | | | |
| | thiomorpholin-1-ylidene}cyanamide | | | |
| 37 | 4-(3-{[6-(2-Hydroxy-2-methylpropoxy)-2- | 1.41E−7 | >1.0E−5 | 2.41E−7 |
| | methylpyridin-3-yl]methyl}-4,4-dimethyl- | | | |
| | 5-oxo-2-thioxoimidazolidin-1-yl)-2- | | | |
| | (trifluoromethyl)benzonitrile | | | |
| 38 | 4-(4,4-Dimethyl-3-{[6-(5-methyl-1H- | 5.87E−8 | >1.0E−5 | 3.66E−7 |
| | tetrazol-1-yl)pyridin-3-yl]methyl}-5-oxo- | | | |
| | 2-thioxoimidazolidin-1-yl)-2- | | | |
| | (trifluoromethyl)benzonitrile | | | |

^{1,2}determined according to assay “Cell-based transactivation assay for wild-type human androgen receptor” described
above
³determined according to assay “Cell-based transactivation assay for human androgen receptor mutant W741L or W741C”
described above
⁴thiohydantoin analog of hydantoin described in example 26 of U.S. patent U.S. RE 35,956. Starting from 4-amino-2-(trifluoromethyl)
benzonitrile and benzylamine, 4-(3-benzyl-4,4-dimethyl-5-oxo-2-thioxoimidazolidin-1-yl)-2-(trifluoromethyl)benzonitrile was prepared
using similar conditions as described in the preparation of Example 1.
¹H-NMR (CDCl₃): 7.98 (d, 1H), 7.94 (d, 1H), 7.83 (dd, 1H), 7.43 (dbr, 2H), 7.37 (m, 2H), 7.35 (m, 1H), 5.14 (s, 2H), 1.45 (s, 6H).
⁵thiohydantoin analog of hydantoin described in example 27 of U.S. patent U.S. RE 35,956. Starting from 4-amino-2-(trifluoromethyl)
benzonitrile and 4-fluorobenzylamine, 4-[3-(4-fluorobenzyl)-4,4-dimethyl-5-oxo-2-thioxoimidazolidin-1-yl]-2-(trifluoromethyl)benzonitrile was
prepared using similar conditions as described in the preparation of Example 1.
¹H-NMR (CDCl₃): 7.98 (d, 1H), 7.93 (d, 1H), 7.82 (dd, 1H), 7.43 (dd, 2H), 7.06 (dd, 2H), 5.10 (s, 2H), 1.46 (s, 6H).
⁶thiohydantoin analog of hydantoin described in example 28 of U.S. patent U.S. RE 35,956. Starting from 4-amino-2-(trifluoromethyl)
benzonitrile and 4-methoxybenzylamine, 4-[3-(4-methoxybenzyl)-4,4-dimethyl-5-oxo-2-thioxoimidazolidin-1-yl]-2-(trifluoromethyl)
benzonitrile was prepared using similar conditions as described in the preparation of Example 1.
¹H-NMR (CDCl₃): 7.97 (d, 1H), 7.93 (d, 1H), 7.81 (dd, 1H), 7.37 (d, 2H), 6.89 (d, 2H), 5.09 (s, 2H), 3.81 (s, 3H), 1.45 (s, 6H).
⁷thiohydantoin analog of hydantoin described in example 29 of U.S. patent U.S. RE 35,956.
Starting from 4-amino-2-(trifluoromethyl)benzonitrile and 4-(trifluoromethyl)benzylamine, 4-{4,4-Dimethyl-5-oxo-2-thioxo-3-[4-
(trifluoromethyl)benzyl]imidazolidin-1-yl}-2-(trifluoromethyl)benzonitrile was prepared using similar conditions as described in the
preparation of Example 1.
¹H-NMR (CDCl₃): 7.99 (d, 1H), 7.94 (d, 1H), 7.83 (dd, 1H), 7.64 (d, 2H), 7.55 (d, 2H), 5.17 (s, 2H), 1.48 (s, 6H).

Table 1 clearly demonstrates that the compounds of the invention have advantageous properties compared to the diarylthiohydantoin compounds disclosed in U.S. patent U.S. RE 35,956. In particular, they show high potency against the androgen receptor (wildtype) paired with little agonistic potency against the androgen receptor (wildtype). Further, the compounds of the invention show a high potency with respect to the inhibition of the mutated androgen receptor W741L.

TABLE 2

| | | E709Y antagon. |
|---|---|---|
| Example | Name of compound | IC50 (mol/l) ¹ |
| Comparative Data; example 12 of US patent US RE 35,956 | 2-(Trifluoromethyl)-4-(3,4,4-trimethyl-5-oxo-2- thioxoimidazolidin-1-yl)benzonitrile 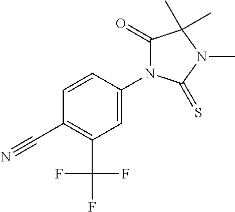 | 1.17E−7 |
| Comparative Data; example 77 of US patent US RE 35,956 | 4-[3-(4-Hydroxybutyl)-4,4-dimethyl-5-oxo-2- thioxoimidazolidin-1-yl]-2-(trifluoromethyl)benzonitrile 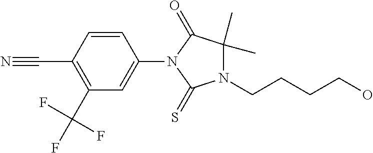 | >1.0E−6 |
| Comparative data ⁴ | 4-(3-Benzyl-4,4-dimethyl-5-oxo-2-thioxoimidazolidin- 1-yl)-2-(trifluoromethyl)benzonitrile 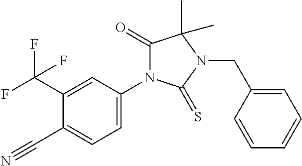 | 5.87E−7 |
| Comparative data⁵ | 4-[3-(4-Fluorobenzyl)-4,4-dimethyl-5-oxo-2- thioxoimidazolidin-1-yl]-2-(trifluoromethyl)benzonitrile 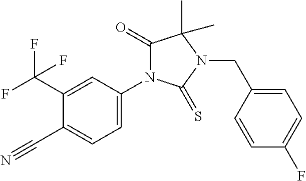 | >1.0E−6 |
| Comparative data ⁶ | 4-[3-(4-Methoxybenzyl)-4,4-dimethyl-5-oxo-2- thioxoimidazolidin-1-yl]-2-(trifluoromethyl)benzonitrile 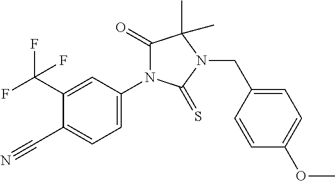 | 8.02E−8 |
| Comparative data ⁷ | 4-{4,4-Dimethyl-5-oxo-2-thioxo-3-[4- (trifluoromethyl)benzyl]imidazolidin-1-yl)}-2- (trifluoromethyl)benzonitrile 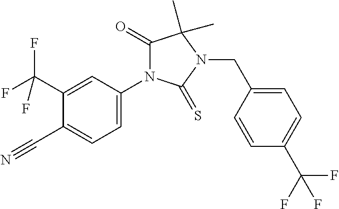 | 4.65E−7 |
| 1 | 4-(3-{[6-(1H-Imidazol-1-yl)pyridin-3-yl]methyl}-4,4- dimethyl-5-oxo-2-thioxoimidazolidin-1-yl)-2- (trifluoromethyl)benzonitrile | 7.21E−9 |
| 2 | 4-(4,4-Dimethyl-5-oxo-2-thioxo-3-{[6- (trifluoromethyl)pyridin-3-yl]methyl}imidazolidin-1- yl)-2-(trifluoromethyl)benzonitrile | 1.42E−8 |
| 3 | 4-[4,4-Dimethyl-3-({6-[2-(morpholin-4- yl)ethoxy]pyridin-3-yl}methyl)-5-oxo-2- thioxoimidazolidin-1-yl]-2-(trifluoromethyl)benzonitrile | 7.95E−8 |
| 4 | 4-(4,4-Dimethyl-3-{[6-(morpholin-4-yl)pyridin-3- yl]methyl}-5-oxo-2-thioxoimidazolidin-1-yl)-2- (trifluoromethyl)benzonitrile | 7.72E−8 |
| 5 | 4-(4,4-Dimethyl-5-oxo-3-{[6-(tetrahydro-2H-pyran-4- yloxy)pyridin-3-yl]methyl}-2-thioxoimidazolidin-1-yl)- 2-(trifluoromethyl)benzonitrile | 4.48E−8 |
| 6 | 4-(3-{[4-Amino-2-(morpholin-4-yl)pyrimidin-5- yl]methyl}-4,4-dimethyl-5-oxo-2-thioxoimidazolidin-1- yl)-2-(trifluoromethyl)benzonitrile | 1.93E−8 |
| 7 | 4-(4,4-Dimethyl-3-{[6-(2-methylmorpholin-4- yl)pyridin-3-yl]methyl}-5-oxo-2-thioxoimidazolidin-1- yl)-2-(trifluoromethyl)benzonitrile | 2.26E−8 |
| 8 | 4-{3-[(6-Methoxypyridin-3-yl)methyl]-4,4-dimethyl-5- oxo-2-thioxoimidazolidin-1-yl}-2- (trifluoromethyl)benzonitrile | 9.18E−9 |
| 9 | 4-(3-{[6-(1-Imino-1-oxido-1λ⁶-thiomorpholin-4- yl)pyridin-3-yl]methyl}-4,4-dimethyl-5-oxo-2- thioxoimidazolidin-1-yl)-2-(trifluoromethyl)benzonitrile | 5.65E−9 |
| 10 | 4-(3-{[6-(2-Hydroxy-2-methylpropoxy)pyridin-3- yl]methyl}-4,4-dimethyl-5-oxo-2-thioxoimidazolidin-1- yl)-2-(trifluoromethyl)benzonitrile | 2.63E−8 |
| 13 | 4-(4,4-Dimethyl-3-{[2-methyl-6- (trifluoromethyl)pyridin-3-yl]methyl}-5-oxo-2- thioxoimidazolidin-1-yl)-2-(trifluoromethyl)benzonitrile | 2.47E−8 |
| 15 | 4-(4,4-Dimethyl-3-{[6-(2-methyl-1H-imidazol-1- yl)pyridin-3-yl]methyl}-5-oxo-2-thioxoimidazolidin-1- yl)-2-(trifluoromethyl)benzonitrile | 2.99E−8 |
| 16 | 4-(3-{[6-(4,4-Dimethyl-2-oxopyrrolidin-1-yl)pyridin-3- yl]methyl}-4,4-dimethyl-5-oxo-2-thioxoimidazolidin-1- yl)-2-(trifluoromethyl)benzonitrile | 1.43E−7 |

¹ determined according to assay “Cell-based transactivation assay for human androgen receptor mutant E709Y” described above
⁴ thiohydantoin analog of hydantoin described in example 26 of US patent US RE 35,956
⁵thiohydantoin analog of hydantoin described in example 27 of US patent US RE 35,956
⁶ thiohydantoin analog of hydantoin described in example 28 of US patent US RE 35,956
⁷ thiohydantoin analog of hydantoin described in example 29 of US patent US RE 35,956

Table 2 demonstrates that the compounds of the invention show high potency with respect to the inhibition of the mutated androgen receptor E709Y.

TABLE 3

| | | W741C antagonism |
|---|---|---|
| Example | Name of compound | IC50 (mol/l) ³ |
| Comparative Data; example 12 of US patent US RE 35,956 | 2-(Trifluoromethyl)-4-(3,4,4-dimethyl-5-oxo-2- thioxoimidazolidin-1-yl)benzonitrile 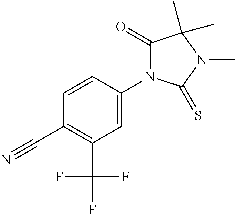 | 2.12E−8 |
| Comparative Data; example 77 of US patent US RE 35,956 | 4-[3-(4-Hydroxybutyl)-4,4-dimethyl-5-oxo-2- thioxoimidazolidin-1-yl]-2-(trifluoromethyl)benzonitrile 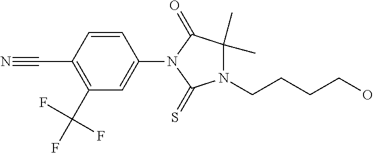 | 2.71E−8 |
| Comparative data ⁴ | 4-(3-Benzyl-4,4-dimethyl-5-oxo-2-thioxoimidazolidin- 1-yl)-2-(trifluoromethyl)benzonitrile 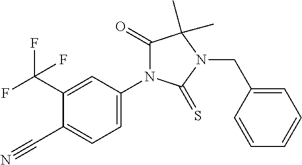 | 1.42E−7 |
| Comparative data⁵ | 4-[3-(4-Fluorobenzyl)-4,4-dimethyl-5-oxo-2- thioxoimidazolidin-1-yl]-2-(trifluoromethyl)benzonitrile 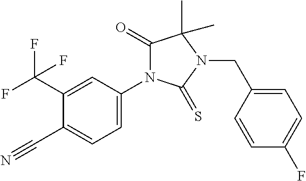 | >1.0E−6 |
| Comparative data ⁷ | 4-{4,4-Dimethyl-5-oxo-2-thioxo-3-[4- (trifluoromethyl)benzyl]imidazolidin-l-yl}-2- (trifluoromethyl)benzonitrile 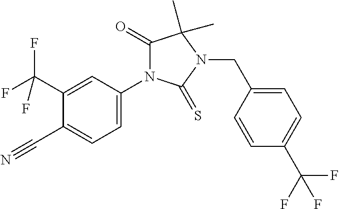 | 7.89E−7 |
| 1 | 4-(3-{[6-(1H-Imidazol-1-yl)pyridin-3-yl]methyl}-4,4- dimethyl-5-oxo-2-thioxoimidazolidin-1-yl)-2- (trifluoromethyl)benzonitrile | 2.16E−7 |
| 4 | 4-(4,4-Dimethyl-3-{[6-(morpholin-4-yl)pyridin-3- yl]methyl}-5-oxo-2-thioxoimidazolidin-1-yl)-2- (trifluoromethyl)benzonitrile | 2.7E−7 |
| 10 | 4-(3-{[6-(2-Hydroxy-2-methylpropoxy)pyridin-3- yl]methyl}-4,4-dimethyl-5-oxo-2-thioxoimidazolidin-1- yl)-2-(trifluoromethyl)benzonitrile | 1.15E−7 |
| 19 | 4-(4,4-Dimethyl-3-{[6-(4-methyl-1H-imidazol-1- yl)pyridin-3-yl]methyl}-5-oxo-2-thioxoimidazolidin-1- yl)-2-(trifluoromethyl)benzonitrile | 5.29E−8 |
| 20 | 4-(4,4-Dimethyl-3-{[6-(1-methyl-1H-pyrazol-5- yl)pyridin-3-yl]methyl}-5-oxo-2-thioxoimidazolidin-1- yl)-2-(trifluoromethyl)benzonitrile | 9.99E−8 |
| 23 | 4-[4,4-Dimethyl-5-oxo-2-thioxo-3-({6-[4- (trifluoromethyl)-1H-imidazol-1-yl]pyridin-3- yl}methyl)imidazolidin-1-yl]-2- (trifluoromethyl)benzonitrile | 1.48E−7 |

³ determined according to assay “Cell-based transactivation assay for human androgen receptor mutant W741L or W741C” described above
⁴ thiohydantoin analog of hydantoin described in example 26 of US patent US RE 35,956
⁵thiohydantoin analog of hydantoin described in example 27 of US patent US RE 35,956
⁷ thiohydantoin analog of hydantoin described in example 29 of US patent US RE 35,956

Table 3 demonstrates that the compounds of the invention show high potency with respect to the inhibition of the mutated androgen receptor W741C.

TABLE 4

| | | Antiproliferative |
|---|---|---|
| | | activity VCaP |
| Example | Name of compound | IC50 (mol/l) ⁸ |
| Comparative Data; example 12 of US patent US RE 35,956 | 2-(Trifluoromethyl)-4-(3,4,4-trimethyl-5-oxo-2- thioxoimidazolidin-1-yl)benzonitrile 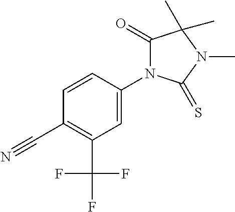 | >1.0E−6 |
| Comparative Data; example 77 of US patent US RE 35,956 | 4-[3-(4-Hydroxybutyl)-4,4-dimethyl-5-oxo-2- thioxoimidazolidin-1-yl]-2-(trifluoromethyl)benzonitrile 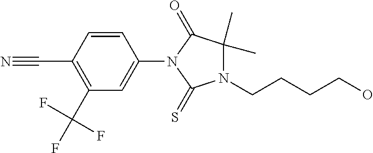 | >1.0E−6 |
| Comparative data ⁴ | 4-(3-Benzyl-4,4-dimethyl-5-oxo-2-thioxoimidazolidin- 1-yl)-2-(trifluoromethyl)benzonitrile 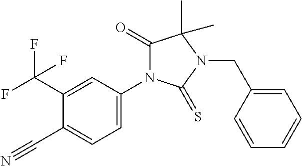 | >1.0E−6 |
| Comparative data⁵ | 4-[3-(4-Fluorobenzyl)-4,4-dimethyl-5-oxo-2- thioxoimidazolidin-1-yl]-2-(trifluoromethyl)benzonitrile 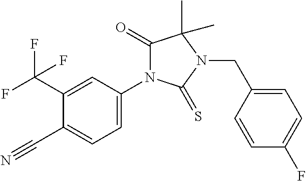 | >1.0E−6 |
| Comparative data ⁶ | 4-[3-(4-Methoxybenzyl)-4,4-dimethyl-5-oxo-2- thioxoimidazolidin-1-yl]-2-(trifluoromethyl)benzonitrile 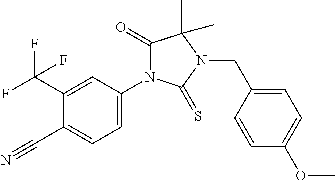 | 8.6E−7 |
| Comparative data ⁷ | 4-{4,4-Dimethyl-5-oxo-2-thioxo-3-[4- (trifluoromethyl)benzyl]imidazolidin-1-yl}-2- (trifluoromethyl)benzonitrile 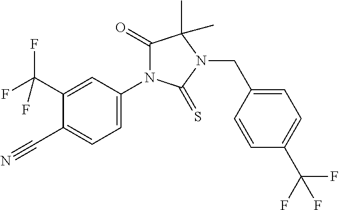 | >1.0E−6 |
| 1 | 4-(3-{[6-(1H-Imidazol-1-yl)pyridin-3-yl]methyl}-4,4- dimethyl-5-oxo-2-thioxoimidazolidin-1-yl)-2- (trifluoromethyl)benzonitrile | 2.91E−7 |
| 2 | 4-(4,4-Dimethyl-5-oxo-2-thioxo-3-{[6- (trifluoromethyl)pyridin-3-yl]methyl}imidazolidin-1- yl)-2-(trifluoromethyl)benzonitrile | 2.47E−7 |
| 3 | 4-[4,4-Dimethyl-3-({6-[2-(morpholin-4- yl)ethoxy]pyridin-3-yl}methyl)-5-oxo-2- thioxoimidazolidin-1-yl]-2-(trifluoromethyl)benzonitrile | 1.76E−7 |
| 4 | 4-(4,4-Dimethyl-3-{[6-(morpholin-4-yl)pyridin-3- yl]methyl}-5-oxo-2-thioxoimidazolidin-1-yl)-2- (trifluoromethyl)benzonitrile | 1.24E−7 |
| 7 | 4-(4,4-Dimethyl-3-{[6-(2-methylmorpholin-4- yl)pyridin-3-yl]methyl}-5-oxo-2-thioxoimidazolidin-1- yl)-2-(trifluoromethyl)benzonitrile | 1.06E−7 |
| 8 | 4-{3-[(6-Methoxypyridin-3-yl)methyl]-4,4-dimethyl-5- oxo-2-thioxoimidazolidin-1-yl}-2- (trifluoromethyl)benzonitrile | 9.25E−8 |
| 10 | 4-(3-{[6-(2-Hydroxy-2-methylpropoxy)pyridin-3- yl]methyl}-4,4-dimethyl-5-oxo-2-thioxoimidazolidin-1- yl)-2-(trifluoromethyl)benzonitrile | 2.29E−7 |
| 13 | 4-(4,4-Dimethyl-3-{[2-methyl-6- (trifluoromethyl)pyridin-3-yl]methyl}-5-oxo-2- thioxoimidazolidin-1-yl)-2-(trifluoromethyl)benzonitrile | 3.0E−7 |
| 15 | 4-(4,4-Dimethyl-3-{[6-(2-methyl-1H-imidazol-1- yl)pyridin-3-yl]methyl}-5-oxo-2-thioxoimidazolidin-1- yl)-2-(trifluoromethyl)benzonitrile | 2.53E−7 |
| 23 | 4-[4,4-Dimethyl-5-oxo-2-thioxo-3-({6-[4- (trifluoromethyl)-1H-imidazol-1-yl]pyridin-3- yl}methyl)imidazolidin-1-yl]-2- (trifluoromethyl)benzonitrile | 3.4E−7 |
| 34 | 4-[3-({6-[4-(Hydroxymethyl)-1H-imidazol-1-yl]pyridin- 3-yl}methyl)-4,4-dimethyl-5-oxo-2-thioxoimidazolidin- 1-yl]-2-(trifluoromethyl)benzonitrile | 3.31E−7 |
| 36 | {4-[5-({3-[4-Cyano-3-(trifluoromethyl)phenyl]-5,5- dimethyl-4-oxo-2-thioxoimidazolidin-1- yl}methyl)pyridin-2-yl]-1-oxido-1λ⁶-thiomorpholin-1- ylidene}cyanamide | 3.29E−7 |
| 37 | 4-(3-{[6-(2-Hydroxy-2-methylpropoxy)-2- methylpyridin-3-yl]methyl}-4,4-dimethyl-5-oxo-2- thioxoimidazolidin-1-yl)-2-(trifluoromethyl)benzonitrile | 3.23E−7 |

⁴ thiohydantoin analog of hydantoin described in example 26 of US patent US RE 35,956
⁵thiohydantoin analog of hydantoin described in example 27 of US patent US RE 35,956
⁶ thiohydantoin analog of hydantoin described in example 28 of US patent US RE 35,956.
⁷ thiohydantoin analog of hydantoin described in example 29 of US patent US RE 35,956
⁸ determined according to assay “Proliferation assay with VCaP cells” described above

Table 4 demonstrates that the compounds of the invention show high antiproliferative activity in VCaP cells.

1. A compound of general formula (I) 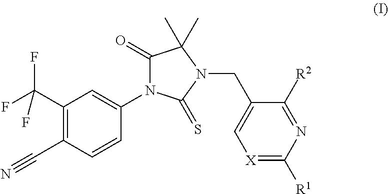
wherein
X means nitrogen or a CH group,
R¹ means a fluorinated C₁-C₃-alkyl-group, an optionally fluorinated C₁-C₄-alkoxy-group, an optionally substituted hydroxy-C₂-C₄-alkoxy-group,
which is substituted with one or two or three substituents selected from the group consisting of methyl, fluorine and trifluoromethyl,
an optionally substituted methoxy-C₂-C₄-alkoxy-group,
which is substituted with one or two or three substituents selected from the group consisting of methyl, fluorine and trifluoromethyl,
a (tetrahydro-2H-pyranyl)oxy-group,
an optionally substituted five membered heteroaromatic group selected from the group consisting of pyrrolyl, pyrazolyl, imidazolyl, triazolyl, tetrazolyl, thienyl, oxazolyl, isoxazolyl, furanyl, thiazolyl, oxadiazolyl,
wherein the five membered heteroaromatic group is substituted with one or two substituents selected from the group consisting of methyl, trifluoromethyl, methoxy-, trifluoromethoxy-, chlorine, fluorine, hydroxy, amino, hydroxymethyl and cyano,
an optionally substituted five, six, or seven membered heterocyclic group selected from pyrrolidinyl, piperidinyl, piperazinyl, morpholinyl, thiomorpholinyl, diazepanyl, tetrahydrofuranyl, pyrrolinyl, imidazolidinyl and oxazepanyl,
wherein the five, six, or seven membered heterocyclic group is substituted with one or two or three substituents selected from the group consisting of methyl, trifluoromethyl, hydroxymethyl, fluorine, hydroxy, oxo, oxido, imino, methylimino, cyanoimino, and cyano;
a residue —O(CH₂)ₙ—Y wherein n=2 or n=3, and Y is an optionally substituted five, six, or seven membered heterocyclic group selected from pyrrolidinyl, piperidinyl, piperazinyl, morpholinyl, thiomorpholinyl, diazepanyl, tetrahydrofuranyl, pyrrolinyl, imidazolidinyl and oxazepanyl,
wherein the five, six, or seven membered heterocyclic group is substituted with one or two substituents selected from the group consisting of methyl, trifluoromethyl, hydroxymethyl, fluorine, hydroxy, oxo, oxido, imino, C₁-C₄-alkylimino-, and cyano; or
a residue —N═S(═O)R³R⁴, wherein R³ represents an aryl group or a phenyl group and R⁴ represents a C₁-C₄-alkyl or a methyl group;
R² means hydrogen, methyl, amino or fluorine,
or their salts, solvates or salts of solvates.

2. The compound according to claim 1, wherein
X means nitrogen or a CH group,
R¹ means a perfluorinated C₁-C₃-alkyl-group,
a C₁-C₄-alkoxy-group,
an optionally substituted hydroxy-C₂-C₄-alkoxy-group,
which is substituted with one or two substituents selected from the group consisting of methyl and fluorine;
a methoxy-C₂-C₄-alkoxy-group,
a (tetrahydro-2H-pyranyl)oxy-group,
an optionally substituted five membered heteroaromatic group selected from the group consisting of pyrazolyl, triazolyl, tetrazolyl, thienyl and imidazolyl,
wherein the five membered heteroaromatic group is substituted with one or two substituents selected from the group consisting of methyl, trifluoromethyl, hydroxymethyl or chlorine,
an optionally substituted five or six or seven membered heterocyclic group selected from pyrrolidinyl, piperidinyl, morpholinyl, thiomorpholinyl, imidazolidinyl, and diazepanyl,
wherein the five or six or seven membered heterocyclic group is substituted with one or two or three substituents selected from the group consisting of methyl, hydroxymethyl, imino, methylimino, cyanoimino, oxido and oxo;
a residue —O(CH₂)ₙ—Y wherein n=2 and Y is a morpholin-4-yl group or a 2-oxoimidazolidin-1-yl group; or
a residue —N═S(═O)R³R⁴, wherein R³ represents a phenyl group and R⁴ represents a methyl group;
R² means hydrogen, methyl, or amino,
or its salts, solvates or salts of solvates.

3. The compound according to claim 1, wherein
X means nitrogen or a CH group,
R¹ means an optionally substituted hydroxy-C₂-C₄-alkoxy-group,
which is substituted with one or two substituents selected from the group consisting of methyl and fluorine;
a methoxy-C₂-C₄-alkoxy-group,
a (tetrahydro-2H-pyranyl)oxy-group,
an optionally substituted five membered heteroaromatic group selected from the group consisting of pyrazolyl, triazolyl, tetrazolyl and imidazolyl,
wherein the five membered heteroaromatic group is substituted with one or two substituents selected from the group consisting of methyl, trifluoromethyl, hydroxymethyl or chlorine;
an optionally substituted five or six membered heterocyclic group selected from pyrrolidinyl, morpholinyl, thiomorpholinyl, imidazolidinyl,
wherein the five or six membered heterocyclic group is substituted with one or two or three substituents selected from the group consisting of methyl, imino, methylimino, cyanoimino, oxido and oxo; or
a residue —O(CH₂)ₙ—Y wherein n=2 and Y is a 2-oxoimidazolidin-1-yl group;
R² means hydrogen or methyl,
or their salts, solvates or salts of solvates.

4. The compound according to claim 1, wherein
X means nitrogen or a CH group,
R¹ means an optionally substituted hydroxy-C₂-C₄-alkoxy-group,
which is substituted with a methyl group;
a (tetrahydro-2H-pyranyl)oxy-group,
an optionally substituted five membered heteroaromatic group selected from the group consisting of pyrazolyl and imidazolyl,
wherein the five membered heteroaromatic group is substituted with one or two substituents selected from the group consisting of methyl, trifluoromethyl, hydroxymethyl or chlorine;
an optionally substituted five or six membered heterocyclic group selected from pyrrolidinyl, morpholinyl, thiomorpholinyl, imidazolidinyl,
wherein the five or six membered heterocyclic group is substituted with one or two or three substituents selected from the group consisting of methyl, imino, methylimino, oxido and oxo; or
a residue —O(CH₂)ₙ—Y wherein n=2 and Y is a 2-oxoimidazolidin-1-yl group;
R² means hydrogen or methyl,
or their salts, solvates or salts of solvates.

5. The compound according to claim 1, wherein
X means a CH group,
R¹ means an optionally substituted hydroxypropoxy-group,
which is substituted with one or two substituents selected from the group consisting of methyl and fluorine; or
an optionally substituted five membered heteroaromatic group selected from the group consisting of pyrazolyl and imidazolyl,
wherein the five membered heteroaromatic group is substituted with one substituent selected from the group consisting of methyl, trifluoromethyl, hydroxymethyl or chlorine,
R² means hydrogen,
or their salts, solvates or salts of solvates.

6. The compound according to claim 1, wherein
X means a CH group,
R¹ means an optionally substituted hydroxypropoxy-group,
which is substituted with a methyl group; or
an optionally substituted imidazolyl group, wherein the imidazolyl group is substituted with a trifluoromethyl group,
R² means hydrogen,
or their salts, solvates or salts of solvates.

7. The compound according to claim 1 selected from
4-(3-{[6-(1H-Imidazol-1-yl)pyridin-3-yl]methyl}-4,4-dimethyl-5-oxo-2-thioxoimidazolidin-1-yl)-2-(trifluoromethyl)benzonitrile,
4-(4,4-Dimethyl-5-oxo-2-thioxo-3-{[6-(trifluoromethyl)pyridin-3-yl]methyl}imidazolidin-1-yl)-2-(trifluoromethyl)benzonitrile,
4-[4,4-Dimethyl-3-({6-[2-(morpholin-4-yl)ethoxy]pyridin-3-yl}methyl)-5-oxo-2-thioxoimidazolidin-1-yl]-2-(trifluoromethyl)benzonitrile,
4-(4,4-Dimethyl-3-{[6-(morpholin-4-yl)pyridin-3-yl]methyl}-5-oxo-2-thioxoimidazolidin-1-yl)-2-(trifluoromethyl)benzonitrile,
4-(4,4-Dimethyl-5-oxo-3-{[6-(tetrahydro-2H-pyran-4-yloxy)pyridin-3-yl]methyl}-2-thioxoimidazolidin-1-yl)-2-(trifluoromethyl)benzonitrile,
4-(3-{[4-Amino-2-(morpholin-4-yl)pyrimidin-5-yl]methyl}-4,4-dimethyl-5-oxo-2-thioxoimidazolidin-1-yl)-2-(trifluoromethyl)benzonitrile,
4-(4,4-Dimethyl-3-{[6-(2-methylmorpholin-4-yl)pyridin-3-yl]methyl}-5-oxo-2-thioxoimidazolidin-1-yl)-2-(trifluoromethyl)benzonitrile,
4-{3-[(6-Methoxypyridin-3-yl)methyl]-4,4-dimethyl-5-oxo-2-thioxoimidazolidin-1-yl}-2-(trifluoromethyl)benzonitrile,
4-(3-{[6-(1-Imino-1-oxido-1λ⁶-thiomorpholin-4-yl)pyridin-3-yl]methyl}-4,4-dimethyl-5-oxo-2-thioxoimidazolidin-1-yl)-2-(trifluoromethyl)benzonitrile,
4-(3-{[6-(2-Hydroxy-2-methylpropoxy)pyridin-3-yl]methyl}-4,4-dimethyl-5-oxo-2-thioxoimidazolidin-1-yl)-2-(trifluoromethyl)benzonitrile,
4-(3-{[6-(2-Methoxyethoxy)pyridin-3-yl]methyl}-4,4-dimethyl-5-oxo-2-thioxoimidazolidin-1-yl)-2-(trifluoromethyl)benzonitrile,
4-(4,4-Dimethyl-3-{[6-(4-methyl-1,4-diazepan-1-yl)pyridin-3-yl]methyl}-5-oxo-2-thioxoimidazolidin-1-yl)-2-(trifluoromethyl)benzonitrile,
4-(4,4-Dimethyl-3-{[2-methyl-6-(trifluoromethyl)pyridin-3-yl]methyl}-5-oxo-2-thioxoimidazolidin-1-yl)-2-(trifluoromethyl)benzonitrile,
4-[3-({6-[4-(Hydroxymethyl)piperidin-1-yl]pyridin-3-yl}methyl)-4,4-dimethyl-5-oxo-2-thioxoimidazolidin-1-yl]-2-(trifluoromethyl)benzonitrile,
4-(4,4-Dimethyl-3-{[6-(2-methyl-1H-imidazol-1-yl)pyridin-3-yl]methyl}-5-oxo-2-thioxoimidazolidin-1-yl)-2-(trifluoromethyl)benzonitrile,
4-(3-{[6-(4,4-Dimethyl-2-oxopyrrolidin-1-yl)pyridin-3-yl]methyl}-4,4-dimethyl-5-oxo-2-thioxoimidazolidin-1-yl)-2-(trifluoromethyl)benzonitrile,
4-(3-{[2-(1H-Imidazol-1-yl)pyrimidin-5-yl]methyl}-4,4-dimethyl-5-oxo-2-thioxoimidazolidin-1-yl)-2-(trifluoromethyl)benzonitrile,
4-(4,4-Dimethyl-3-{[6-(1-methyl-1H-pyrazol-4-yl)pyridin-3-yl]methyl}-5-oxo-2-thioxoimidazolidin-1-yl)-2-(trifluoromethyl)benzonitrile,
4-(4,4-Dimethyl-3-{[6-(4-methyl-1H-imidazol-1-yl)pyridin-3-yl]methyl}-5-oxo-2-thioxoimidazolidin-1-yl)-2-(trifluoromethyl)benzonitrile,
4-(4,4-Dimethyl-3-{[6-(1-methyl-1H-pyrazol-5-yl)pyridin-3-yl]methyl}-5-oxo-2-thioxoimidazolidin-1-yl)-2-(trifluoromethyl)benzonitrile,
4-(3-{[6-(4-Chloro-2-methyl-1H-imidazol-1-yl)pyridin-3-yl]methyl}-4,4-dimethyl-5-oxo-2-thioxoimidazolidin-1-yl)-2-(trifluoromethyl)benzonitrile,
4-[4,4-Dimethyl-3-({6-[1-(methylimino)-1-oxido-1λ⁶-thiomorpholin-4-yl]pyridin-3-yl}methyl)-5-oxo-2-thioxoimidazolidin-1-yl]-2-(trifluoromethyl)benzonitrile,
4-[4,4-Dimethyl-5-oxo-2-thioxo-3-({6-[4-(trifluoromethyl)-1H-imidazol-1-yl]pyridin-3-yl}methyl)imidazolidin-1-yl]-2-(trifluoromethyl)benzonitrile,
4-(4,4-Dimethyl-5-oxo-3-{[6-(thien-2-yl)pyridin-3-yl]methyl}-2-thioxoimidazolidin-1-yl)-2-(trifluoromethyl)benzonitrile,
4-(4,4-Dimethyl-5-oxo-3-{[6-(2-oxoimidazolidin-1-yl)pyridin-3-yl]methyl}-2-thioxoimidazolidin-1-yl)-2-(trifluoromethyl)benzonitrile,
4-{4,4-Dimethyl-3-[(6-{[methyl(oxido)phenyl-λ⁶-sulfanylidene]amino}pyridin-3-yl)methyl]-5-oxo-2-thioxoimidazolidin-1-yl}-2-(trifluoromethyl)benzonitrile,
4-(4,4-Dimethyl-3-{[6-(5-methyl-1H-pyrazol-1-yl)pyridin-3-yl]methyl}-5-oxo-2-thioxoimidazolidin-1-yl)-2-(trifluoromethyl)benzonitrile,
4-(3-{[6-(2,2-Difluoro-3-hydroxypropoxy)pyridin-3-yl]methyl}-4,4-dimethyl-5-oxo-2-thioxoimidazolidin-1-yl)-2-(trifluoromethyl)benzonitrile,
4-(4,4-Dimethyl-5-oxo-2-thioxo-3-{[6-(1H-1,2,3-triazol-1-yl)pyridin-3-yl]methyl}imidazolidin-1-yl)-2-(trifluoromethyl)benzonitrile,
4-(4,4-Dimethyl-5-oxo-2-thioxo-3-{[6-(2H-1,2,3-triazol-2-yl)pyridin-3-yl]methyl}imidazolidin-1-yl)-2-(trifluoromethyl)benzonitrile,
4-(4,4-Dimethyl-5-oxo-3-{[6-(1H-tetrazol-1-yl)pyridin-3-yl]methyl}-2-thioxoimidazolidin-1-yl)-2-(trifluoromethyl)benzonitrile,
4-(3-{[6-(4,5-Dichloro-1H-imidazol-1-yl)pyridin-3-yl]methyl}-4,4-dimethyl-5-oxo-2-thioxoimidazolidin-1-yl)-2-(trifluoromethyl)benzonitrile,
4-[4,4-Dimethyl-5-oxo-2-thioxo-3-({6-[3-(trifluoromethyl)-1H-1,2,4-triazol-1-yl]pyridin-3-yl}methyl)imidazolidin-1-yl]-2-(trifluoromethyl)benzonitrile,
4-[3-({6-[4-(Hydroxymethyl)-1H-imidazol-1-yl]pyridin-3-yl}methyl)-4,4-dimethyl-5-oxo-2-thioxoimidazolidin-1-yl]-2-(trifluoromethyl)benzonitrile,
4-[4,4-Dimethyl-5-oxo-3-({6-[2-(2-oxoimidazolidin-1-yl)ethoxy]pyridin-3-yl}methyl)-2-thioxoimidazolidin-1-yl]-2-(trifluoromethyl)benzonitrile,
{4-[5-({3-[4-Cyano-3-(trifluoromethyl)phenyl]-5,5-dimethyl-4-oxo-2-thioxoimidazolidin-1-yl}methyl)pyridin-2-yl]-1-oxido-1 thiomorpholin-1-ylidene}cyanamide,
4-(3-{[6-(2-Hydroxy-2-methylpropoxy)-2-methylpyridin-3-yl]methyl}-4,4-dimethyl-5-oxo-2-thioxoimidazolidin-1-yl)-2-(trifluoromethyl)benzonitrile,
4-(4,4-Dimethyl-3-{[6-(5-methyl-1H-tetrazol-1-yl)pyridin-3-yl]methyl}-5-oxo-2-thioxoimidazolidin-1-yl)-2-(trifluoromethyl)benzonitrile,
or its salts, solvates or salts of solvates.

8. Method for the preparation of the compounds according to claim 1, in which method an intermediate compound of general formula (2) 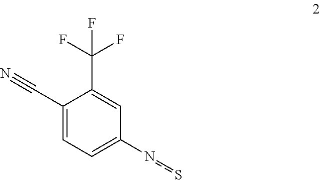
is reacted with a compound of general formula (5) 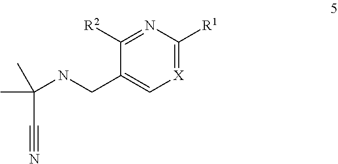
in which X, R¹ and R² are as defined for the compound of general formula (I), thus providing a compound of general formula (6) 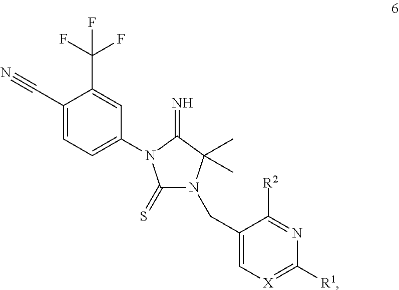
which is then hydrolyzed to the compounds of general formula (I) and the resulting compounds of the formula (I) are optionally reacted with the corresponding (i) solvents and/or (ii) bases or acids to the solvates, salts and/or solvates of the salts thereof.

9. (canceled)

10. Composition comprising a compound as defined in claim 1 in combination with at least one or more further active ingredients.

11. Composition according to claim 10, in which the at least one or more further active ingredients is selected from LHRH (luteinizing hormone-releasing hormone) agonists,
LHRH (luteinizing hormone-releasing hormone) antagonists,
C(17,20)-lyase inhibitors,
5-alpha-reductase inhibitors type I,
5-alpha-reductase inhibitors type II,
cytostatic agents,
VEGF (Vascular Endothelial Growth Factor)-Kinase inhibitors
antigestagens,
antiestrogens,
EGF Antibodies,
estrogens, or
other androgen receptor antagonists.

12. A pharmaceutical composition comprising a compound as defined in claim 1 in combination with one or more inert, nontoxic, pharmaceutically suitable adjuvants.

13. (canceled)

14. (canceled)

15. Method of treating a hyper-proliferative disorder in a mammal, which comprises administering to a mammal in need of such treatment an effective amount of a compound according to claim 1.

16. The method of claim 15, wherein the hyper-proliferative disorder is prostate cancer.

17. The method of claim 15, wherein the hyper-proliferative disorder is castration-resistant prostate cancer.
